SEQUENCE LISTING (111 sequences)

SEQ ID NO: 1 - protein, 382 aa, Zea mays
MPCSSPAPTWLLRVSPAAAAADQAAASSSCSSKGGGRVLTAGTTTMDTAATAAAGGNAADLQESSSSGQSRLAARGHWRPAEDAKLRELVALYGPQNWNLIAEKLDGRSGKSCRLRWFNQLDPRISKRPFSDEEEERLMAAHRFYGNKWAMIARLFPGRTDNAVKNHWHVIMARKYREQSTAYRRRKLNQAVQRKLEAASAAVAMPPGAGAGDVAVGQHHHLLAAAAAAHAHDAAYSFAADPYGFGIRHQYCTFPFPPGAASAEDPPPPTQIHPFCLFPGPSSAAAHADSRRLPWPPSSDAPGVARYGEPHQLLQLPVQSGWIDGVGVAAAGHHEPPFVLGNNGGAAAFEGTTRQQGSGAHFEAAAAPPPPAFIDFLGVGAT

SEQ ID NO: 2 - protein, 337 aa, Zea mays
MPPTSRRAAAAASPGSRRAATGAQRRTPSSVSSSRCTGPRTGTSSPRSWTADPVRASIIIILHCGKSCRLRWFNQLDPRISKRPFSDEEEERLMAAHRFYGNKWAMIARLFPGRTDNAVKNHWHVIMARKYREQSTAYRRRKLNQAVQRKLEAASAAVAMPPGAGAGDVAVGQHHHLLAAAAAAHAHDAAYSFAADPYGFGIRHQYCTFPFPPGAASAEDPPPPTQIHPFCLFPGPSSAAAHADSRRLPWPPSSDAPGVARYGEPHQLLQLPVQSGWIDGVGVAAAGHHEPPFVLGNNGGAAAFEGTTRQQGSGAHFEAAAAPPPPAFIDFLGVGAT

SEQ ID NO: 3 - protein, 305 aa, Zea mays
MPCSSPAPTWLLRVSPAAAAADQAAASSSCSSKGGGRVLTAGTTTMDTAATAAAGGNAADLQESSSSGQSRLAARGHWRPAEDAKLRELVALYGPQNWNLIAEKLDGRSGKSCRLRWFNQLDPRISKRPFSDEEEERLMAAHRFYGNKWAMIARLFPGRTDNAVKNHWHVIMARKYREQSTAYRRRKLNQAVQRKLEAASAAVAMPPGAGAGDVAVGQHHHLLAAAAAAHAHDAAYSFAADPYGFGIRHQYCTFPFPPGAASAEDPPPPTQIHPFCLFPGEHSGNPYPARHYLLARHRTRTPRMG

SEQ ID NO: 4 - protein, 283 aa, Zea mays
MAYYCNACCAGKSCRLRWFNQLDPRISKRPFSDEEEERLMAAHRFYGNKWAMIARLFPGRTDNAVKNHWHVIMARKYREQSTAYRRRKLNQAVQRKLEAASAAVAMPPGAGAGDVAVGQHHHLLAAAAAAHAHDAAYSFAADPYGFGIRHQYCTFPFPPGAASAEDPPPPTQIHPFCLFPGPSSAAAHADSRRLPWPPSSDAPGVARYGEPHQLLQLPVQSGWIDGVGVAAAGHHEPPFVLGNNGGAAAFEGTTRQQGSGAHFEAAAAPPPPAFIDFLGVGAT

SEQ ID NO: 5 - protein, 296 aa, Zea mays
MPCSSPAPTWLLRVSPAAAAADQAAASSSCSSKGGGRVLTAGTTTMDTAATAAAGGNAADLQESSSSGQSRLAARGHWRPAEDAKLRELVALYGPQNWNLIAEKLDGRSEIIVIIDEERTGFDEMEAMALASLFWKRRRQPNAKTERRQRLELCKQGRAAASAGSTSTPGSASAPSATRRRSAWLRTASTATSGPSRASSPAARTTPRTTGTSSWRASTASSPRPTAAASSTRQSSGSSRQPPPRSQCRRARARETSPSASTTTCWPPPRRPTPTTPPTASPRTPTASASATNTAP

SEQ ID NO: 6 - protein, 390 aa, Sorghum bicolor
MPCSSAAPTWLLRVASAADQASSSSSSKGGGRVLTAGTTGTTMDTAATAAAAGNAADLQESSSSGQSRLAARGHWRPAEDAKLRELVALYGPQNWNLIAEKLDGRSGKSCRLRWFNQLDPRISKRPFSDEEEERLMAAHRFYGNKWAMIARLFPGRTDNAVKNHWHVIMARKYREQSTAYRRRKLNQAVQRKLEASAAAVATMPPAAGSTGDVVGAALGHHHHQLLAAAAAAAHDAAYGFAAADPYGAFGFRQYYPFPPASAEDTPPPPPPPFCLFPGPSSAAALHADSRRLPWPSSSSSDAAAAAAGGGRYGEPQQQLLLPVVHGGSWIDGVGVAVAGGHHEAQFVLGNNGGAFEGTTRQQGAAAGAHFEAAAAAPPPAFIDFLGVGAT

SEQ ID NO: 7 - protein, 369 aa, Oryza sativa
MPCTSAAWMLHVGGAAAEQASSSSSSKGGGRVVTAGTTTMDTGGYNNGGGGGGGGGNGRGVGDHQESSSSGGGGGQSSRLAARGHWRPAEDAKLRELVALYGPQNWNLIADKLDGRSGKSCRLRWFNQLDPRISKRPFSDEEEERLMAAHRFYGNKWAMIARLFPGRTDNAVKNHWHVIMARKYREQSTAYRRRKLNQAVQRKLDATTASDVVVAHHHPYAAAHDPYAFTFRHYCFPFPAASPAAADEPPFTCLFPGTAATAGRGGVGGMTWPDAMAAGEVIDDGAGGGRYVVAEPPPPFLVPAAPHGWLGGHEMMVMVNDGGDVAAGVASSYDGMIGRDQGGGGSHFEAAAAAAAAPAFIDFLGVGAT

SEQ ID NO: 8 - protein, 330 aa, Arabidopsis thaliana
MEMVHADVASLSITPCFPSSLSSSSHHHYNQQQHCIMSEDQHHSMDQTTSSDYFSLNIDNAQHLRSYYTSHREEDMNPNLSDYSNCNKKDTTVYRSCGHSSKASVSRGHWRPAEDTKLKELVAVYGPQNWNLIAEKLQGRSGKSCRLRWFNQLDPRINRRAFTEEEEERLMQAHRLYGNKWAMIARLFPGRTDNSVKNHWHVIMARKFREQSSSYRRRKTMVSLKPLINPNPHIFNDFDPTRLALTHLASSDHKQLMLPVPCFPGYDHENESPLMVDMFETQMMVGDYIAWTQEATTFDFLNQTGKSEIFERINEEKKPPFFDFLGLGTV

SEQ ID NO: 9 - protein, 402 aa, Glycine max
AKSFPLQFHRVSNKHYFSHIPNRPTSSSSSTTTTTKFVTQIHEMKSWPAARVVFADMMGSLSLATVSNNASSSQESNVYGYGYGYASGVGNGSTSDLVGAGESNNSNEKTNHNNGKFSEEESNPNENHANGKEVDSGHSKLCARGHWRPAEDSKLKELVALYGPQNWNLIAEKLEGRSGKSCRLRWFNQLDPRINRRAFSEEEEERLMQAHRIYGNKWAMIARLFPGRTDNAVKNHWHVIMARKYREQSSAYRRRRMSQSVHRRVEQNPTFFGSNGSPQNMTSGREAMPNTTHVGLSAQAQQQAPFDFFSGGGSNDIVLESISHMRSRERTNGSHNHHCQLSGCYPHYPQQYLMAMQQQLDNNNNFYSFLNSSPAASTAREPSSSPYGVPPPFFDFLGVGAT

SEQ ID NO: 10 - DNA, 8918 bp, Zea mays
  source 1..8918: mol_type = unassigned DNA
attctaattaatacttacttagtgtaaacacgttaacccctcgaccgtagcttcgactattgcggttctttttctcgcgtaaccgtagaagtgctctctattttatattgctcgcttttataatattatatattgtatggtgtaatgttcttatgcaaatatattattgggggccttccccttccgaaggtcctaaaaacataattaaccatttggctttagcatgaactattacaggaagcttcgtctctaggagataagcctctttctaatgacgaaagacacacatgatgaagatagatctaaagaagacaagagtaaacgccgaagctaatagcggacataaatagctgaagaaggaaaacggaggaatgctgataatggctgaagaaggaaaagactatttggtcctttataatttgtattacgatcatgtgtaaacattaagaacataaatgaacttttgctcgggctgcgtcccgtgcctataaatagatgaacagtaacatcgtactgttcaggctgaattgtattcactctctcgcatcctcgccttcaacaagccgaaggtactaatgtaatattattattatagatattcatatatgttttatggaatgaaagaataaaagaattattatgatttaactatcatatttatttccttgtatccctatctttgtattgatatgatgaaggtgtgtccttcctgaccttcgtccgaagagtattacacccgtaaggagataatacttcgagggacgaaggtcctttacgattaacaattgtgttgccttgttcttgacttacaccatttgagaacaagtgaccaacatttggcgcccacctccggtgaactcacttccacaaccttcggcaagcatcaaccttcgacatgccgccgaagaaggcaacgatgccagccactggacacaaaccaggacgcactctccttgcgcgaggctaggaaccaaaagaggaaggccactagcccaactcttcaaaaggaccagcttgaccaggagatcagggatttagaagcaatccatcaacaggtgcaaagaaaaagtgagaaaatgctccggctggccgatcttcagaagaagattgacgacgcagctgaggagatgcgtcatcttactcaagatggccaagatcgaaggcctcagcacagggagcttcgtcaggagagctcattcaacgaagatgaatggtacaatgactttcatcatggtaactttacttttgatgatgcttctcctctggtggcagaattgcaggctaccccgtggccataatcttacaagccaccttagttgcccatgtatgatgggcactcggatccaaagcaatttctgatgagttacgaggcaacaatatcctcgtacgggggcaacgctactatcatggcaaagttcttcgtcatggcagtcagaagcgtggcctagacatggtattcctcccttagatcagggacaatcacatcatggcagaagctgaaggatatgctggtcactagtttccagggctttcagacaaagccaattattgctcaggccttgttccagtgcacgcaagaccaggaggagtacctgtaggcttacgtccgaaggttcctacgtttgagagctcaatcgcctatagtgcccaatgaaattgtcattgaggccatgattaaggggcttcggccaggacctacaaccaaatatttcgctaggaagcccccacaaaccctggagaagttccttaagaagatggatgagtacatctgggtcgataatgatttccgccaaagaagggaggaagcatacaagttttctgagatgaccaggggcttcggaggaggacttcatcccaggcatatcaggtcaatccataactccaatgctaacgatgaaaggcccaatagtgctcagagcggccatcatcgctcacagtcttcgagcatgcagcaaacttcctataggccaccagctccgagaggcagaggaggaagaagcttcagtggaggaagattcggtaatcaacccaggaagttgtattgcctcttctgtgacgaggttaagggccacacaacaaggacgtgtcaggtcacaatccagaagcaaaaggaaattgttgaagccgaggcatggcagaaccagccgaagcaagtcctttatactgcttcgtgctactctccatacatcccagaatatgtaggcaaccaatagactacagcttcaccaagtcactcccaagcttcctgggcccaattactgccacccccaccaatggtgcctgccccaagccatgataagcagccagaagggcacctttggcctcaacaacaacgtgatcttcgggatcagtctgaggttcgcacagttaacagtactgtacctgaggccaggcacatctactgaagatgacacatcgttttggtcaaaagaaagtcctgatatgtcccatttctactattttttgctttcatatttctgttgcaaaagacaatatagtaaggtttaacattcaacttgatgtaataaacctatcgttacaccatcgagtgtgacaaaatcaagttcctaagctccaaagacgttcctaagggagcgcagagtaagttctgaagctcaaaagtcgtttctaagggaatacagagccaaaatgccacctaagtaaaaggtgaagagactccaaatcactcctaagggaatgcagagcctgaatgccacctaagtaacaggtgaagaagttcaaaagtcgttcctaaggggatgcagagctgaaatactacctaagtaaagggtgaagagactccaaagtcgttcctaaggggacgcaaagtctggatgtgtattcgtgtaggttttttttaccttcggcataaatattattttgcatcataccatcataacatatcgcatagcattgtatcatacatcattttgcatcagcaaaaggctatggagaagaagggaaattgctccttcgcaacatgtatcttcggtggatataatttactacacgaagcccaccttcgtcaacatctttgagcaaactcaatgttttatactcgaacaaaatatattgagatagattttccattcttcgtgggaacgccaagctgattcgaggtgtttagatatttgatttattagttctgcggagcacaaaaggcttttgcctacatggtagtagatgatgttatttatgaacagcagccctcaactcaagtgatgcattatgatattattattattattatcgggagttttggagacacaataatgtcgctgggtgtggacgaacgaagccgtgcgtgcgtgcgtgcgccgggggcagagcggcagcggcacagtgcgcggggcctgcgcccccccgtgcagttgaaaagataggtgcctgtagtggttgcagggctcattcagacagcactggagggcatgcatgttctgtcaaggcatcagggctccgcgcctgggagatagatcatctcatctactctactactagtctactacccaggcaactcaaagcaatgcaagagagcctgtttattgttgggactcgtaccccgcccagcagctctgcacatttaacaatcccactcatgcttttgtttcttattattattattattattacaaaaaaaaaagaaaataggccgaacctgctgcctgaaacccgcatgcaccacctgcaggggcctctatttaatttgcgctggtctcgtacttgacgttgcgtcctgctgctcctttgagattcctgctggaagattgcgatctctgccttcttttcttttcttctttttttttaaaaaaaacaaaaggcatcagtttgagtacttttattggctaagtacgaaaacattaactcccggtcaagagaaaggtggtgtgtgtttgtgcgtgcgtgcgtgtgtgtttaataaggcccagcagcctccctgagctggtcgttttatatggccagtcaagcgttgcagagtagtatattgtctatgcattactacttgactaagcagccactgaactctgcacaggtctacttggccctcagagtacacattattcaccggccagtcgtcgtgcaaagagcacagtttctgttaccgctgatgattggatgccgtaattaatagccggttcccattcccgtccagatttccatcgcgattcgcgaggaaaagcgtccgtgtgtgtgtacacgcgcttcaaactgttgggcgcatgtacacgtacgtacgctcggacgtaccgatccactgttgagagtgaaaactgaatggggagggggagagagagaagaaagagaaagagaccagtagcaagtagtagtagatcgacgatcaggcaggcgggctacagtgctaaccttctctctctctatctctctctggccgtgcgtcgtccttgcaagccacacatgtggtgagatgacatctacacgtgcggtggggacgcccgatgctttgttaaactcggtgaaagccatgagagatggcagcgggcggtgtggtggtagtagtagacagctgggacagtgacaagccttgcgtgcagtaagatcttctgcgcccttaccttaattacccctcctccgcctccagtttttaccgcgcgaaccctagtaaaataactcccgccagtgtgctctctctctctctctacgactatttttcaccccttctttcccagttccgtgccttgcacttcgccttttcaaaaagcttttgccatatgcagtacacatgtgttaatagagtagtaactttcttttcttttgcaaactgattgagatccaaagcaagcaagcaagcaagctgtatgtacttgcaaacaacacccttggtagctatccccctcccgtgcccgtgcccgtgcccgtgccctgcaatccccgacccggagcagcagccaccaccggcgcggcgtcccgcgagccagcacagacgatccctccccattcccgcccgcactgcccagcaccaggaggagcagctagcctatccaacagtgaaaagcacacacgcgttccggactccggactacgcccggccctcccctcccctccccctcctcctgcggtttttagacaggggagtgcgtgcgcccgagcgatccgtccatctgacgggaatgagagggtgcgtgcgtgcgtgggggagagtgagatgcctgcctccctgtagcgtgtaggagtagctctggcctcttcctctacctccagccgtgcggttttctgctgcggaagaaacgggagcagtgtcgctcgtcccgctcgcgcgcacatcctcaactcgtctccgtctctcccgcggcaactgacgacgatgccgtgctcgtcgccggccccgacatggctgctgcgagtgtcgccagcggctgccgcggccgaccaggccgccgcctcgtcctcgtgctcatccaagggcggaggccgcgtgctcacggccggtaccaccaccatggacacggccgccaccgctgccgccggcggcaatgccgccgacctccaggagagcagcagcagcggccagtcccggctcgcggcgcgcggccactggcgcccagcggaggacgccaagctccgtgagctcgtcgcgctgtacgggccccagaactggaacctcatcgccgagaagctggacggcagatccggtacgcgcgtccatcatcatcatcttgcattgtggtaatctcatctgactgacctcagcctcaggctcagctgcaccgatcatccatccccatacaaagacactgtatacatacattttaacaacaacaaggtttagctccgcatggcctattactgcaatgcttgctgcgcagggaagagctgccgcctccgctggttcaaccagctagacccccggatcagcaagcgccccttcagcgacgaggaggaggagcgcctgatggctgcgcaccgcttctacggcaacaagtgggccatgatcgcgcgcctcttccccggccgcacggacaacgccgtgaagaaccactggcacgtcatcatggcgcgcaagtaccgcgagcagtccacggcctaccgccgccgcaagctcaaccaggcagtccagcggaagctcgaggcagcctccgccgcggtcgcaatgccgccgggcgcgggcgcgggagacgtcgccgtcggccagcaccaccacctgctggccgccgccgcggcggcccacgcccacgacgccgcctacagcttcgccgcggacccctacggcttcggcatccgccaccaatactgcaccttcccgttcccgccaggcgccgcttcggctgaggacccgccgccgccaacgcaaatacatcccttctgcttgttccctggtgagcactccggcaatccctacccggcccgccattacctgctagcacgacaccgcactcgcacgccacggatgggatgatgtatgtatgacaggcgggcccctgctggcacagtaactcccggctgattgttgccccccaccgagtccggccggcaacactggctaacagacgagctcgagaagctcgcggagcccatctatgagcgagtgcgacggggactggtctagtgttctcctagccgccagtgatggatcatggattttcttttatttttactgtgacctagccgggctttgtttacagtgttcgtgtgacgagacctatctatctacctgagggctgaggctgaggggaggggctagctgtacctgtacggttgcggctgctgcgtttgcactgtagagggtggaggatgatcgatcgatcgaggagaggagtccgcacggttcggcacacagtacattcaggcaggcaggcaggcgcacagcatcgatgaaccgtgattccatgatagggagagacacggggacgaatcaatggaggcctcgatcgggcggtgcactgcagcacggcctccgacccgtccgatcccgccgcagcgtgggtagccaggcacgcacagccttgctcgccaccggcccctggcgggcgtaaatgcctttgcttgaaataggaagccctgcgggcgagatctcgtcggcatcggagaccagaccaggcacgcacaggccgcgccggccgggaagactttggactttgagcacggcgccacagtggcgcgcactgcatgtcgcacagtgccgcacacgctcgctcgctgccaagaggtgagcagagcagagcagcagtgaggggctttagatgacgtgccaaggcgatgcccgccatcaatgcgcgagggcgctacctgcctgccccttgttgctcacctgctgctgtgctgtgccttctgttctcgcatacaacatatctcctgtgtgctgtgctgtgctgtgcgtgcgttttcttttcttggagggttctgtttgcttcttaattccctgctcagctgattactattggtttcttgattgtgtgccatcgcggcgtcgctttcctgctggtctctaacttccaagatgcaattaatcagtcgctagtaacaccactgtctctctctctcacgcgaccttccaatctgcttgtcttctgatccatgccaaacagggcccagcagcgcggcggcgcacgccgacagcaggcgccttccctggccgccgtcgtcggacgcgcccggcgtcgcccggtacggggagccgcatcagctcctgcagctgcccgttcaaagcggctggatcgacggcgtcggcgtggccgcggccggccaccacgagccgcccttcgtcttgggcaacaacgggggcgcggccgcctttgaagggacgacaagacagcagggctccggcgctcactttgaagctgccgcggcgccgccgccgccagcgttcatagatttcctcggggtcggagccacatgaacgcgcgcgcatgtgcatgcatgctcgacttcaccacgcgccttgcagttgcatgtagtgggatctagctaggacaggtagctcgagcagcctctatctagtatctagctagctacctttgccatatatgaggagtaagtacatcgatccaaggcttaggtttaagcttatgtagtagcactatgtatgtatatgtatgtctgaatgtctctggctagataggaactagtgacggagattgagcttatttatttcggggctttgtagagggagggatgcttggactttgatcatttgggatcggattagttacttgattagcccccagccatatatgtatgtatgcaagcaagatagatcgtcgatgatggtgacttgcagagctaaatttgcaattatccattcttttactagccaagagtagaagctagtagatttgcagatgggatagccgagtatacggggttttcttttttgctgcatagaagagtctgcttggttttaactttttttttttttgcaaggggagactataaataccatctaaaccagtcatttctcatatgtggagctcggaaacattgttagggagttgatactctaatcctttggctctagacacctaagtgctaaaagaatcagtgattagcattgatgattcgtaaagtgcttagattagttagaccgttatcataacgtttgctctaggtttgtttctagttgttagaagtgaggttagacattctcaacgcattagtgcttatgtgcaccattgtattccttaacgttatgcatgccattgtattgtaccgagtggggttgtaacttgtaagatcacatcaactacgtctgtggtgtagccttcactatgtactggaggaaacgagactaatgacatttcagcaagaatctcgacagtgagcacctgatagagatagaagcggagcaccacttgcacatggagaaggctcgtggctatctatagagcttggtcatcgcatgggctaccgtttcatggactctaagaaggactagtaagaagcttgcaccttattgataccttggt

SEQ ID NO: 11 - DNA, 2135 bp, Zea mays
  source 1..2135: mol_type = unassigned DNA
agcaagcaagctgtatgtacttgcaaacaacacccttggtagctatccccctcccgtgcccgtgcccgtgcccgtgccctgcaatccccgacccggagcagcagccaccaccggcgcggcgtcccgcgagccagcacagacgatccctccccattcccgcccgcactgcccagcaccaggaggagcagctagcctatccaacagtgaaaagcacacacgcgttccggactccggactacgcccggccctcccctcccctccccctcctcctgcggtttttagacaggggagtgcgtgcgcccgagcgatccgtccatctgacgggaatgagagggtgcgtgcgtgcgtgggggagagtgagatgcctgcctccctgtagcgtgtaggagtagctctggcctcttcctctacctccagccgtgcggttttctgctgcggaagaaacgggagcagtgtcgctcgtcccgctcgcgcgcacatcctcaactcgtctccgtctctcccgcggcaactgacgacgatgccgtgctcgtcgccggccccgacatggctgctgcgagtgtcgccagcggctgccgcggccgaccaggccgccgcctcgtcctcgtgctcatccaagggcggaggccgcgtgctcacggccggtaccaccaccatggacacggccgccaccgctgccgccggcggcaatgccgccgacctccaggagagcagcagcagcggccagtcccggctcgcggcgcgcggccactggcgcccagcggaggacgccaagctccgtgagctcgtcgcgctgtacgggccccagaactggaacctcatcgccgagaagctggacggcagatccgggaagagctgccgcctccgctggttcaaccagctagacccccggatcagcaagcgccccttcagcgacgaggaggaggagcgcctgatggctgcgcaccgcttctacggcaacaagtgggccatgatcgcgcgcctcttccccggccgcacggacaacgccgtgaagaaccactggcacgtcatcatggcgcgcaagtaccgcgagcagtccacggcctaccgccgccgcaagctcaaccaggcagtccagcggaagctcgaggcagcctccgccgcggtcgcaatgccgccgggcgcgggcgcgggagacgtcgccgtcggccagcaccaccacctgctggccgccgccgcggcggcccacgcccacgacgccgcctacagcttcgccgcggacccctacggcttcggcatccgccaccaatactgcaccttcccgttcccgccaggcgccgcttcggctgaggacccgccgccgccaacgcaaatacatcccttctgcttgttccctgggcccagcagcgcggcggcgcacgccgacagcaggcgccttccctggccgccgtcgtcggacgcgcccggcgtcgcccggtacggggagccgcatcagctcctgcagctgcccgttcaaagcggctggatcgacggcgtcggcgtggccgcggccggccaccacgagccgcccttcgtcttgggcaacaacgggggcgcggccgcctttgaagggacgacaagacagcagggctccggcgctcactttgaagctgccgcggcgccgccgccgccagcgttcatagatttcctcggggtcggagccacatgaacgcgcgcgcatgtgcatgcatgctcgacttcaccacgcgccttgcagttgcatgtagtgggatctagctaggacaggtagctcgagcagcctctatctagtatctagctagctacctttgccatatatgaggagtaagtacatcgatccaaggcttaggtttaagcttatgtagtagcactatgtatgtatatgtatgtctgaatgtctctggctagataggaactagtgacggagattgagcttatttatttcggggctttgtagagggagggatgcttggactttgatcatttgggatcggattagttacttgattagcccccagccatatatgtatgtatgcaagcaagatagatcgtcgatgatggtgacttgcagagctaaatttgcaattatccattcttttactagccaagagtagaagctagtagatttgcagatgggatagccgagtatacggggt

SEQ ID NO: 12 - DNA, 2098 bp, Zea mays
  source 1..2098: mol_type = unassigned DNA
agcaagcaagctgtatgtacttgcaaacaacacccttggtagctatccccctcccgtgcccgtgcccgtgcccgtgccctgcaatccccgacccggagcagcagccaccaccggcgcggcgtcccgcgagccagcacagacgatccctccccattcccgcccgcactgcccagcaccaggaggagcagctagcctatccaacagtgaaaagcacacacgcgttccggactccggactacgcccggccctcccctcccctccccctcctcctgcggtttttagacaggggagtgcgtgcgcccgagcgatccgtccatctgacgggaatgagagggtgcgtgcgtgcgtgggggagagtgagatgcctgcctccctgtagcgtgtaggagtagctctggcctcttcctctacctccagccgtgcggttttctgctgcggaagaaacgggagcagtgtcgctcgtcccgctcgcgcgcacatcctcaactcgtctccgtctctcccgcggcaactgacgacgatgccgtgctcgtcgccggccccgacatggctgctgcgagtgtcgccagcggctgccgcggccgaccaggccgccgcctcgtcctcgtgctcatccaagggcggaggccgcgtgctcacggccggtaccaccaccatggacacggccgccaccgctgccgccggcggcaatgccgccgacctccaggagagcagcagcagcggccagtcccggctcgcggcgcgcggccactggcgcccagcggaggacgccaagctccgtgagctcgtcgcgctgtacgggccccagaactggaacctcatcgccgagaagctggacggcagatccggtacgcgcgtccatcatcatcatcttgcattgtgggaagagctgccgcctccgctggttcaaccagctagacccccggatcagcaagcgccccttcagcgacgaggaggaggagcgcctgatggctgcgcaccgcttctacggcaacaagtgggccatgatcgcgcgcctcttccccggccgcacggacaacgccgtgaagaaccactggcacgtcatcatggcgcgcaagtaccgcgagcagtccacggcctaccgccgccgcaagctcaaccaggcagtccagcggaagctcgaggcagcctccgccgcggtcgcaatgccgccgggcgcgggcgcgggagacgtcgccgtcggccagcaccaccacctgctggccgccgccgcggcggcccacgcccacgacgccgcctacagcttcgccgcggacccctacggcttcggcatccgccaccaatactgcaccttcccgttcccgccaggcgccgcttcggctgaggacccgccgccgccaacgcaaatacatcccttctgcttgttccctgggcccagcagcgcggcggcgcacgccgacagcaggcgccttccctggccgccgtcgtcggacgcgcccggcgtcgcccggtacggggagccgcatcagctcctgcagctgcccgttcaaagcggctggatcgacggcgtcggcgtggccgcggccggccaccacgagccgcccttcgtcttgggcaacaacgggggcgcggccgcctttgaagggacgacaagacagcagggctccggcgctcactttgaagctgccgcggcgccgccgccgccagcgttcatagatttcctcggggtcggagccacatgaacgcgcgcgcatgtgcatgcatgctcgacttcaccacgcgccttgcagttgcatgtagtgggatctagctaggacaggtagctcgagcagcctctatctagtatctagctagctacctttgccatatatgaggagtaagtacatcgatccaaggcttaggtttaagcttatgtagtagcactatgtatgtatatgtatgtctgaatgtctctggctagataggaactagtgacggagattgagcttatttatttcggggctttgtagagggagggatgcttggactttgatcatttgggatcggattagttacttgattagcccccagccatatatgtatgtatgcaagcaagatagatcgtcgatgatggtgacttgcagagctaaatttgcaa

SEQ ID NO: 13 - DNA, 1758 bp, Zea mays
  source 1..1758: mol_type = unassigned DNA
agcaagcaagctgtatgtacttgcaaacaacacccttggtagctatccccctcccgtgcccgtgcccgtgcccgtgccctgcaatccccgacccggagcagcagccaccaccggcgcggcgtcccgcgagccagcacagacgatccctccccattcccgcccgcactgcccagcaccaggaggagcagctagcctatccaacagtgaaaagcacacacgcgttccggactccggactacgcccggccctcccctcccctccccctcctcctgcggtttttagacaggggagtgcgtgcgcccgagcgatccgtccatctgacgggaatgagagggtgcgtgcgtgcgtgggggagagtgagatgcctgcctccctgtagcgtgtaggagtagctctggcctcttcctctacctccagccgtgcggttttctgctgcggaagaaacgggagcagtgtcgctcgtcccgctcgcgcgcacatcctcaactcgtctccgtctctcccgcggcaactgacgacgatgccgtgctcgtcgccggccccgacatggctgctgcgagtgtcgccagcggctgccgcggccgaccaggccgccgcctcgtcctcgtgctcatccaagggcggaggccgcgtgctcacggccggtaccaccaccatggacacggccgccaccgctgccgccggcggcaatgccgccgacctccaggagagcagcagcagcggccagtcccggctcgcggcgcgcggccactggcgcccagcggaggacgccaagctccgtgagctcgtcgcgctgtacgggccccagaactggaacctcatcgccgagaagctggacggcagatccgggaagagctgccgcctccgctggttcaaccagctagacccccggatcagcaagcgccccttcagcgacgaggaggaggagcgcctgatggctgcgcaccgcttctacggcaacaagtgggccatgatcgcgcgcctcttccccggccgcacggacaacgccgtgaagaaccactggcacgtcatcatggcgcgcaagtaccgcgagcagtccacggcctaccgccgccgcaagctcaaccaggcagtccagcggaagctcgaggcagcctccgccgcggtcgcaatgccgccgggcgcgggcgcgggagacgtcgccgtcggccagcaccaccacctgctggccgccgccgcggcggcccacgcccacgacgccgcctacagcttcgccgcggacccctacggcttcggcatccgccaccaatactgcaccttcccgttcccgccaggcgccgcttcggctgaggacccgccgccgccaacgcaaatacatcccttctgcttgttccctggtgagcactccggcaatccctacccggcccgccattacctgctagcacgacaccgcactcgcacgccacggatgggatgatgtatgtatgacaggcgggcccctgctggcacagtaactcccggctgattgttgccccccaccgagtccggccggcaacactggctaacagacgagctcgagaagctcgcggagcccatctatgagcgagtgcgacggggactggtctagtgttctcctagccgccagtgatggatcatggattttcttttatttttactgtgacctagccgggctttgtttacagtgttcgtgtgacgagacctatctatctacctgagggctgaggctgaggggaggggctagctgtacctgtacggttgcggctgctgcgtttgcac

SEQ ID NO: 14 - DNA, 2331 bp, Zea mays
  source 1..2331: mol_type = unassigned DNA
agcaagcaagctgtatgtacttgcaaacaacacccttggtagctatccccctcccgtgcccgtgcccgtgcccgtgccctgcaatccccgacccggagcagcagccaccaccggcgcggcgtcccgcgagccagcacagacgatccctccccattcccgcccgcactgcccagcaccaggaggagcagctagcctatccaacagtgaaaagcacacacgcgttccggactccggactacgcccggccctcccctcccctccccctcctcctgcggtttttagacaggggagtgcgtgcgcccgagcgatccgtccatctgacgggaatgagagggtgcgtgcgtgcgtgggggagagtgagatgcctgcctccctgtagcgtgtaggagtagctctggcctcttcctctacctccagccgtgcggttttctgctgcggaagaaacgggagcagtgtcgctcgtcccgctcgcgcgcacatcctcaactcgtctccgtctctcccgcggcaactgacgacgatgccgtgctcgtcgccggccccgacatggctgctgcgagtgtcgccagcggctgccgcggccgaccaggccgccgcctcgtcctcgtgctcatccaagggcggaggccgcgtgctcacggccggtaccaccaccatggacacggccgccaccgctgccgccggcggcaatgccgccgacctccaggagagcagcagcagcggccagtcccggctcgcggcgcgcggccactggcgcccagcggaggacgccaagctccgtgagctcgtcgcgctgtacgggccccagaactggaacctcatcgccgagaagctggacggcagatccggtacgcgcgtccatcatcatcatcttgcattgtggtaatctcatctgactgacctcagcctcaggctcagctgcaccgatcatccatccccatacaaagacactgtatacatacattttaacaacaacaaggtttagctccgcatggcctattactgcaatgcttgctgcgcagggaagagctgccgcctccgctggttcaaccagctagacccccggatcagcaagcgccccttcagcgacgaggaggaggagcgcctgatggctgcgcaccgcttctacggcaacaagtgggccatgatcgcgcgcctcttccccggccgcacggacaacgccgtgaagaaccactggcacgtcatcatggcgcgcaagtaccgcgagcagtccacggcctaccgccgccgcaagctcaaccaggcagtccagcggaagctcgaggcagcctccgccgcggtcgcaatgccgccgggcgcgggcgcgggagacgtcgccgtcggccagcaccaccacctgctggccgccgccgcggcggcccacgcccacgacgccgcctacagcttcgccgcggacccctacggcttcggcatccgccaccaatactgcaccttcccgttcccgccaggcgccgcttcggctgaggacccgccgccgccaacgcaaatacatcccttctgcttgttccctgggcccagcagcgcggcggcgcacgccgacagcaggcgccttccctggccgccgtcgtcggacgcgcccggcgtcgcccggtacggggagccgcatcagctcctgcagctgcccgttcaaagcggctggatcgacggcgtcggcgtggccgcggccggccaccacgagccgcccttcgtcttgggcaacaacgggggcgcggccgcctttgaagggacgacaagacagcagggctccggcgctcactttgaagctgccgcggcgccgccgccgccagcgttcatagatttcctcggggtcggagccacatgaacgcgcgcgcatgtgcatgcatgctcgacttcaccacgcgccttgcagttgcatgtagtgggatctagctaggacaggtagctcgagcagcctctatctagtatctagctagctacctttgccatatatgaggagtaagtacatcgatccaaggcttaggtttaagcttatgtagtagcactatgtatgtatatgtatgtctgaatgtctctggctagataggaactagtgacggagattgagcttatttatttcggggctttgtagagggagggatgcttggactttgatcatttgggatcggattagttacttgattagcccccagccatatatgtatgtatgcaagcaagatagatcgtcgatgatggtgacttgcagagctaaatttgcaattatccattcttttactagccaagagtagaagctagtagatttgcagatgggatagccgagtatacggggttttcttttttgctgcatagaag

SEQ ID NO: 15 - DNA, 2907 bp, Zea mays
  variation 676: note = n = a, t, c, or g
  source 1..2907: mol_type = unassigned DNA
ggcccagcagcagtgtgagcaacaccgacaccagtgaagagatggcacctgcagtttcagcaaaggtgtccacgctgcgtcgcgggagcaggccaaggaaccacaatgtgaagatttctgggcctgaatgggtgaatgtgtgagtgggttgttacggcccagtgttgggttggccagtggggcgtgtattaaacgaacgctgtctgttttggagagggcaagaaatatgaaaaacctgaactagcttcttcctgcccaagttttcctcctctgctccttctacttttcctatctatcttcttccattccggtgtatctagcagatagttaacaattgccagcggaccatcttcagcatgttcgggcagttttggaattgttggttcgtgataagtggcaggttaagctgtcgaaatgctcatttgcgcagcagcagttatcatatttaggacacatcatttctgctgcaggcgttgcaactgatccatcgaagattcaagcggtggcatcgtgggctaccccacgttcaatcaaggaattgcgcagttttttgggcttggcgggttattaccggcggtttgtcaggcacttcggtattctggccaagcctttgacaaccctcctgaagaaaggatctatgtttgtgtggacttctcagcatacagcagcatttcangcgctgaaggatgccttagtctctgctccggtcttggctttgccaaactttgctctgccgttctgtttggaaacggatgcaagcaaccagggagtgggcgctgttttgatgcaagggggtcatccaatcgcttatttgagtaaggcattggggccaaaatctcaagggctttctacttatgagaaggaagttttggcgatccttactgcagttgatcattggcggcattatctgcaactgaaggagttccatattgtcacggaccatcgcagtttggctcagctggatgaacagcgtcttcatactccatggcagaaaaagatgttttctcgtctgttgggccttcaataccgtatcatgtacaagaaaggctctgaaaatggagcagccgatgcactgtccaggcatcctcagttgacacagacatgtctcgctgtgtcctcttgcacaccgcagtggcttacaaatattatggattcgtattctcacgatgatatgtcacgagaaataatagctaagttggcagttgacgcagctgcagttccacattactcttggcatgacggtcttttgcgttacaagaatcggatttgggtggggtcagatattcccttacagactcggctgattgaggcttttcattcaacggctgtcggtggtcattcgggtattccagtgacgtatgctcgcttgaagcagctgttcgcttggcgcggaatgaaaaaagttgttcaagaatttgtcagtcaatgcattgtgtgtcagcgcgcgaaggccgatcgtgctcgtttgcctggtctgttacagcctctaccggttcccacgtccttatggcagatcatatccctggattttattgaagcattaccgcgttctcaatcctttacatgcatcctcgtggttgtggatatgtttaccaaatatgcaaattttttgcctctgcgacatccatacacagctctctcggtggcaaagttattccatgatcaggtatataaacaccatggtttacctcaatctattgtttctgatcgtgatagaatatttctcagtaatctatggagggagctgtttcgtctggctgatgtgcagctaagaatgagctcggcttatcaccctcagaccgatggccaaacggagcgtgtgaatcagtgtttggaaacttttttgcgctgttttgtgcatgcctgtccgaatcagtggagtcagtggttatctgtagctcaattctggtataattcttctcctcactcagctattggtcggtctccattcgaagccttgtatggttgtcgtcccaggttttttggcattgatcatgacactatcatcagtaccactgatctggcttcatggttgcatgagcggcagttgatgtctgatgtcatcaaactgcatttggacagggccaaacttcgtatgaaacgtcaagcggacaaaggtcgttccgagcggcactttgcagtaggcgattgggtgttcctcaagctgcagccatatattcagtccactatcgctgttcgtgccagccagaagctgtcattcagattttttgggccttttcaagtgatacaacgtgttgggctggtagcgtataaacttcaactccctccatcttctcgtgtgcatccagtttttcatgtatctcagttaaaaaaggctgtgggcactggaatggtggtttctccttctcttccttctgataatttcatgttcagtgtgcctgaaaagtttttgcagcgacgtcaaatttttaagggcgataaatcagttcagcaggtgttggtgcagtggtctttcatgccagaggagcttgctacttgggaggatgttgagaatttacagcagcagtttcctgatgcagctgtctggggacatccagcagccttaggaggggggaatgtgagcaacaccgacaccagtgaagagatggcacctgcagtttcagcaaaggtgtccacgctgcgtcgcgggagcaggccaaggaaccacaatgtgaagatttctgggcctgaatgggtgaatgtgtgagtgggttgttacggcccagtgttgggttggccagtggggcgtgtattaaacgaacgctgtctgttttggagagggcaagaaatatgaaaaacctgaactagcttcttcctgcccaagttttcctcctctgctccttctacttttcctatctatcttcttccattccggtgtatctagcagatagttaacagcgcggcggc

SEQ ID NO: 16 - DNA, 1290 bp, Zea mays
  source 1..1290: mol_type = unassigned DNA
atgccgtgctcgtcgccggccccgacatggctgctgcgagtgtcgccagcggctgccgcggccgaccaggccgccgcctcgtcctcgtgctcatccaagggcggaggccgcgtgctcacggccggtaccaccaccatggacacggccgccaccgctgccgccggcggcaatgccgccgacctccaggagagcagcagcagcggccagtcccggctcgcggcgcgcggccactggcgcccagcggaggacgccaagctccgtgagctcgtcgcgctgtacgggccccagaactggaacctcatcgccgagaagctggacggcagatccgagataattgtcattatagacgaagagcggacgggattcgacgaaatggaggcgatggcgttggcttctctgttctggaaacgcagacgacagccaaacgccaaaacggaaaggagacagcgcttggagctgtgtaaacaggggaagagctgccgcctccgctggttcaaccagctagacccccggatcagcaagcgccccttcagcgacgaggaggaggagcgcctgatggctgcgcaccgcttctacggcaacaagtgggccatgatcgcgcgcctcttccccggccgcacggacaacgccgtgaagaaccactggcacgtcatcatggcgcgcaagtaccgcgagcagtccacggcctaccgccgccgcaagctcaaccaggcagtccagcggaagctcgaggcagcctccgccgcggtcgcaatgccgccgggcgcgggcgcgggagacgtcgccgtcggccagcaccaccacctgctggccgccgccgcggcggcccacgcccacgacgccgcctacagcttcgccgcggacccctacggcttcggcatccgccaccaatactgcaccttcccgttcccgccaggcgccgcttcggctgaggacccgccgccgccaacgcaaatacatcccttctgcttgttccctgggcccagcagcgcggcggcgcacgccgacagcaggcgccttccctggccgccgtcgtcggacgcgcccggcgtcgcccggtacggggagccgcatcagctcctgcagctgcccgttcaaagcggctggatcgacggcgtcggcgtggccgcggccggccaccacgagccgcccttcgtcttgggcaacaacgggggcgcggccgcctttgaagggacgacaagacagcagggctccggcgctcactttgaagctgccgcggcgccgccgccgccagcgttcatagatttcctcggggtcggagccacatga

SEQ ID NO: 17 - DNA, 1173 bp, Sorghum bicolor
  source 1..1173: mol_type = unassigned DNA
atgccgtgctcgtcggcggccccgacgtggctgctgcgggtggcgtcggcggccgaccaggcctcgtcctcgtcctcgtccaagggcggcggccgcgtgctcaccgccggcaccactggcaccaccatggacacggccgccaccgctgccgccgccggcaatgccgccgacctccaggagagcagcagcagcgggcagtcccggctcgcggcgcgcggccactggcgccccgccgaggacgccaagctccgcgagctcgtcgcgctctacggtccccagaactggaacctcatcgccgagaagctcgacggcagatccgggaagagctgccgcctccggtggttcaaccagctggacccgcggatcagcaagcggcccttcagcgacgaggaagaagagcggctgatggcggcgcaccgcttctacggcaacaagtgggcgatgatcgcgcgcctgttcccggggcgcacggacaacgccgtcaagaaccactggcacgtcatcatggcgcgcaagtaccgcgagcagtccacggcgtaccgccgccgcaagctcaaccaggcagtccagcggaagctcgaggcctccgccgcggcggtcgcaacaatgccgccggccgcgggcagcacgggagacgtcgtcggcgccgccctcggccaccaccaccaccaactcctggcggccgccgccgccgccgcccacgacgcggcctacggcttcgccgcggcggacccctacggcgccttcggcttccgccaatactacccgttcccgccagcttcggccgaggacacgccgccgccgccgccgcctcccttctgcttgttccctgggcccagcagcgcggcggcgcttcacgccgacagcaggcgccttccctggccgtcgtcgtcgtcgtcggatgctgccgctgccgccgccggtggcggcaggtacggggagccgcagcagcagctcctgctgcccgttgttcacggtggcagctggatcgacggcgtcggcgtggccgtggccggcggtcaccacgaggcgcagttcgtcttgggcaacaacgggggagcctttgaagggaccacaagacagcagggcgccgccgccggcgctcactttgaagctgccgcggcggcgccgccgccagcgttcatagatttcctcggtgtcggagccacatga

SEQ ID NO: 18 - DNA, 1110 bp, Oryza sativa
  source 1..1110: mol_type = unassigned DNA
atgccgtgcacgtcggcggcgtggatgttgcacgtgggtggtgcggcggcggagcaggcgtcgtcgtcgtcgtcgtccaagggaggggggagggtggtgacggcggggacgacgacgatggacacgggcgggtacaataatggaggtggtggtgggggtgggggtggcaatggcggcggcggcggcgaccaccaggagagcagcagcagcggcggcggcggcgggcagtcatccaggctcgcggcgcgcggccactggcgccccgccgaggacgccaagctccgcgagctcgtcgcgctctacggcccccagaactggaacctcatcgccgacaagctcgacggcagatccgggaagagctgcaggctgaggtggttcaaccagctggacccgaggatcagcaagaggccgttcagcgacgaggaggaggagaggctgatggcggcgcacaggttctacggcaacaagtgggcgatgatcgcgcgcctcttccccggccggaccgacaacgccgtcaagaaccactggcacgtcatcatggcgcgcaagtacagggagcagtccaccgcctaccgacgccgcaagctcaaccaggccgtccagcgcaagctcgacgccaccaccgcctccgacgtcgtcgtcgcccaccaccacccctacgccgccgcccacgacccctacgccttcaccttccgccactactgcttcccttttccggccgcctcccccgccgccgccgacgagccgcccttcacctgcttgttccccgggacggcggcgacggccgggcgcggcggcggcggtggcatgacatggccggacgccatggccgctggcgaggtcatcgacgacggcgccggcggcggccggtacgtcgtggccgagccgccgccgccgttcctggtgccggcggcgccgcacgggtggctcggaggccacgagatgatggtgatggtgaacgacggcggcgacgtcgccgccggcgtggcgtcgtcgtacgacgggatgataggcagggatcagggcggcggcggctcacacttcgaggcggctgcggcggcagcggcggcgccggcattcatagacttcctcggggttggcgccacatga

SEQ ID NO: 19 - DNA, 993 bp, Arabidopsis thaliana
  source 1..993: mol_type = unassigned DNA
atggagatggtgcatgctgacgtggcgtctctctccataacaccttgcttcccgtcttctttgtcttcgtcctcacatcatcactataaccaacaacaacattgtatcatgtcggaagatcaacaccattcgatggatcagaccacttcatcggactacttctctttaaatatcgacaatgctcaacatctccgtagctactacacaagtcatagagaagaagacatgaaccctaatctaagtgattacagtaattgcaacaagaaagacacaacagtctatagaagctgtggacactcgtcaaaagcttcggtgtctagaggacattggagaccagctgaagatactaagctcaaagaactagtcgccgtctacggtccacaaaactggaacctcatagctgagaagctccaaggaagatccgggaaaagctgtaggcttcgatggtttaaccaactagacccaaggataaatagaagagccttcactgaggaagaagaagagaggctaatgcaagctcataggctttatggtaacaaatgggcgatgatagcgaggcttttccctggtaggactgataattctgtgaagaaccattggcatgttataatggctcgcaagtttagggaacaatcttcttcttaccgtaggaggaagacgatggtttctcttaagccactcattaaccctaatcctcacattttcaatgattttgaccctacccggttagctttgacccaccttgctagtagtgaccataagcagcttatgttaccagttccttgcttcccaggttatgatcatgaaaatgagagtccattaatggtggatatgttcgaaacccaaatgatggttggcgattacattgcatggacacaagaggcaactacattcgatttcttaaaccaaaccgggaagagtgagatatttgaaagaatcaatgaggagaagaaaccaccatttttcgattttcttgggttggggacggtgtga

SEQ ID NO: 20 - DNA, 1209 bp, Glycine max
  source 1..1209: mol_type = unassigned DNA
gcaaaatcttttcctcttcaatttcaccgtgtgtcaaacaaacattacttctctcatatacccaacaggccaacatcatcatcatcatcaacaacaacaacaacaaaatttgtgacacaaattcatgaaatgaagtcatggccagcagcaagagtagtctttgcagatatgatggggtctctctctcttgccacagtttcaaataatgcttcttcatctcaagaaagtaatgtttatggttatggttatggttatgcctctggggtgggaaatgggagcacaagtgatttagtaggggctggggagagtaacaacagtaatgagaaaacaaaccataataatggcaagttcagtgaagaagaaagtaaccctaatgagaaccatgccaatggcaaagaggtggatagtggacactccaaactttgtgcaagagggcactggaggcctgctgaagattccaagctcaaggagcttgtggctctttatggccctcagaattggaacctcatagctgaaaaactagaaggaagatcaggtaagagttgtaggctgaggtggttcaaccagttggatccaaggatcaacagaagagcattcagtgaagaagaagaagagaggctaatgcaggcacatagaatttacggcaacaaatgggccatgatcgcaaggcttttccctgggagaacagataatgcagtgaagaatcactggcatgttataatggccaggaagtacagggaacaatccagtgcttacaggaggaggagaatgagccaatctgtgcacagaagagtggagcaaaatccaaccttttttggctcaaatggttctccacaaaacatgactagtgggagagaagcaatgccaaacaccacacatgttggcttgtctgctcaagctcaacaacaagcaccatttgatttcttctctggtggtggaagcaatgacatagtgttagagtccataagccatatgagaagcagggaaagaaccaatggatctcacaatcatcattgccaattatctggttgctaccctcattatccccaacaatatttgatggcaatgcaacaacagctagacaacaacaacaatttttacagtttcttgaattcctcaccagcagcatcaacagctagggaaccatcatcatcaccatatggtgttccacccccattttttgacttccttggagtaggagccacatga

SEQ ID NO: 21 - DNA, 1427 bp, Zea mays
  source 1..1427: mol_type = unassigned DNA
tgcggtttttagacaggggagtgcgtgcgcccgagcgatccgtccatctgacgggaatgagagggtgcgtgcgtgcgtgggggagagtgagatgcctgcctccctgtagcgtgtaggagtagctctggcctcttcctctacctccagccgtgcggttttctgctgcggaagaaacgggagcagtgtcgctcgtcccgctcgcgcgcacatcctcaactcgtctccgtctctcccgcggcaactgacgacgatgccgtgctcgtcgccggccccgacatggctgctgcgagtgtcgccagcggctgccgcggccgaccaggccgccgcctcgtcctcgtgctcatccaagggcggaggccgcgtgctcacggccggtaccaccaccatggacacggccgccaccgctgccgccggcggcaatgccgccgacctccaggagagcagcagcagcggccagtcccggctcgcggcgcgcggccactggcgcccagcggaggacgccaagctccgtgagctcgtcgcgctgtacgggccccagaactggaacctcatcgccgagaagctggacggcagatccggtacgcgcgtccatcatcatcatcttgcattgtggtaatctcatctgactgacctcagcctcaggctcagctgcaccgatcatccatccccatacaaagacactgtatacatacattttaacaacaacaaggtttagctccgcatggcctattactgcagagataattgtcattatagacgaagagcggacgggattcgacgaaatggaggcgatggcgttggcttctctaggcgatggcgttggcttctctgttctggaaacgcagacgacagccaaacgccaaaacggaaaggagacagcgcttggagctgtgtaaacaggtatgcttgctgcgcagggaagagctgccgcctccgctggttcaaccagctagacccccggatcagcaagcgccccttcagcgacgaggaggaggagcgcctgatggctgcgcaccgcttctacggcaacaagtgggccatgatcgcgcgcctcttccccggccgcacggacaacgccgtgaagaaccactggcacgtcatcatggcgcgcaagtaccgcgagcagtccacggcctaccgccgccgcaagctcaaccaggcagtccagcggaagctcgaggcagcctccgccgcggtcgcaatgccgccgggcgcgggcgcgggagacgtcgccgtcggccagcaccaccacctgctggccgccgccgcggcggcccacgcccacgacgccgcctacagcttcgccgcggacccctacggcttcggcatccgccaccaatactgcaccttcccgttcccgccaggcgccgcttcggctgaggacccgccgccgccaacgcaaatacatcccttctgcttgttccctg

SEQ ID NO: 22 - DNA, 25 bp, Zea mays
  source 1..25: mol_type = unassigned DNA
aagctcaaccaggcagtccagcgga

SEQ ID NO: 23 - DNA, 32 bp, Zea mays
  source 1..32: mol_type = unassigned DNA
caccatgccgtgctcgtcgccggccccgacat

SEQ ID NO: 24 - DNA, 29 bp, Zea mays
  source 1..29: mol_type = unassigned DNA
caccatggcctattactgcaatgcttgct

SEQ ID NO: 25 - DNA, 27 bp, Zea mays
  source 1..27: mol_type = unassigned DNA
ctgctggcacagtaactcccggctgat

SEQ ID NO: 26 - DNA, 25 bp, Zea mays
  source 1..25: mol_type = unassigned DNA
tgagcaacaaggggcaggcaggtag

SEQ ID NO: 27 - DNA, 24 bp, Zea mays
  source 1..24: mol_type = unassigned DNA
tcatgtggctccgaccccgaggaa

SEQ ID NO: 28 - DNA, 25 bp, Zea mays
  source 1..25: mol_type = unassigned DNA
ccagcacagacgatccctccccatt

SEQ ID NO: 29 - DNA, 25 bp, Zea mays
  source 1..25: mol_type = unassigned DNA
tgcggtgtcgtgctagcaggtaatg

SEQ ID NO: 30 - DNA, 32 bp, Zea mays
  source 1..32: mol_type = unassigned DNA
agagaagccaacgccawcgcctcyatttcgtc

SEQ ID NO: 31 - DNA, 20 bp, Zea mays
  source 1..20: mol_type = unassigned DNA
aggcgatggcgttggcttct

SEQ ID NO: 32 - DNA, 20 bp, Zea mays
  source 1..20: mol_type = unassigned DNA
gctcattcagacagcactgg

SEQ ID NO: 33 - DNA, 17 bp, Zea mays
  source 1..17: mol_type = unassigned DNA
gatcgcaatcttccagc

SEQ ID NO: 34 - DNA, 17 bp, Zea mays
  source 1..17: mol_type = unassigned DNA
ggatcagcaagcgcccc

SEQ ID NO: 35 - DNA, 17 bp, Zea mays
  source 1..17: mol_type = unassigned DNA
gctgcgcaccgcttcta

SEQ ID NO: 36 - DNA, 19 bp, Zea mays
  source 1..19: mol_type = unassigned DNA
gaagggacgacaagacagc

SEQ ID NO: 37 - DNA, 18 bp, Zea mays
  source 1..18: mol_type = unassigned DNA
gcgttcatagatttcctc

SEQ ID NO: 38 - DNA, 20 bp, Zea mays
  source 1..20: mol_type = unassigned DNA
cctcttctgtgacgaggtta

SEQ ID NO: 39 - DNA, 20 bp, Zea mays
  source 1..20: mol_type = unassigned DNA
tggagacacaataatgtcgc

SEQ ID NO: 40 - DNA, 20 bp, Zea mays
  source 1..20: mol_type = unassigned DNA
agttccgtgccttgcacttc

SEQ ID NO: 41 - DNA, 17 bp, Zea mays
  source 1..17: mol_type = unassigned DNA
gatcgaccgcaagacgg

SEQ ID NO: 42 - DNA, 18 bp, Zea mays
  source 1..18: mol_type = unassigned DNA
agtcggagcggtgcgtgc

SEQ ID NO: 43 - protein, 1379 aa, Zea mays
MSSSDPEEIRARVVVLGSPHADGGDEWARPELEAFHLPSPAHQPPGFLAGQPEAAEQPTLPAPAGRSSSSSNTPTTSAGGGAAPPPPSSPPPPPASLETEQPPNARPASAGANDSKKPTPPAALRDLFRFADGLDCALMLIGTLGALVHGCSLPVFLRFFADLVDSFGSHADDPDTMVRLVVKYAFYFLVVGAAIWASSWAEISCWMWTGERQSTRMRIRYLDAALRQDVSFFDTDVRASDVIYAINADAVVVQDAISEKLGNLIHYMATFVAGFVVGFTAAWQLALVTLAVVPLIAVIGGLSAAALAKLSSRSQDALSGASGIAEQALAQIRIVQAFVGEEREMRAYSAALAVAQRIGYRSGFAKGLGLGGTYFTVFCCYGLLLWYGGHLVRAQHTNGGLAIATMFSVMIGGLALGQSAPSMAAFAKARVAAAKIFRIIDHRPGISSRDGAEPESVTGRVEMRGVDFAYPSRPDVPILRGFSLSVPAGKTIALVGSSGSGKSTVVSLIERFYDPSAGQILLDGHDLRSLELRWLRRQIGLVSQEPALFATSIRENLLLGRDSQSATLAEMEEAARVANAHSFIIKLPDGYDTQVGERGLQLSGGQKQRIAIARAMLKNPAILLLDEATSALDSESEKLVQEALDRFMIGRTTLVIAHRLSTIRKADVVAVLQGGAVSEMGAHDELMAKGENGTYAKLIRMQEQAHEAALVNARRSSARPSSARNSVSSPIMTRNSSYGRSPYSRRLSDFSTSDFTLSIHDPHHHHRTMADKQLAFRAGASSFLRLARMNSPEWAYALAGSIGSMVCGSFSAIFAYILSAVLSVYYAPDPRYMKREIAKYCYLLIGMSSAALLFNTVQHVFWDTVGENLTKRVREKMFAAVLRNEIAWFDADENASARVAARLALDAQNVRSAIGDRISVIVQNSALMLVACTAGFVLQWRLALVLLAVFPLVVGATVLQKMFMKGFSGDLEAAHARATQIAGEAVANLRTVAAFNAERKITGLFEANLRGPLRRCFWKGQIAGSGYGVAQFLLYASYALGLWYAAWLVKHGVSDFSRTIRVFMVLMVSANGAAETLTLAPDFIKGGRAMRSVFETIDRKTEVEPDDVDAAPVPERPRGEVELKHVDFSYPSRPDIQVFRDLSLRARAGKTLALVGPSGCGKSSVLALVQRFYEPTSGRVLLDGKDVRKYNLRALRRVVAVVPQEPFLFAASIHENIAYGREGATEAEVVEAAAQANAHRFIAALPEGYRTQVGERGVQLSGGQRQRIAIARALVKQAAIVLLDEATSALDAESERCVQEALERAGSGRTTIVVAHRLATVRGAHTIAVIDDGKVAEQGSHSHLLKHHPDGCYARMLQLQRLTGAAAGPGPSTSCNGAA

SEQ ID NO: 44 - DNA, 8199 bp, Zea mays
  source 1..8199: mol_type = unassigned DNA
tttgaaaagactgaatgaattgtgtgcgtcaaggatcggagagggattcagaacataagtgatggaccctctttcttagatgattatgtatccagtctttatcaatgcatgatgctgaagcgacaaaacctagacatatgtctttcaggtgaggctctgcacagatctgaaactgagttactgttcatctttgatgttgagagacagacgaactctccacatctgatgcaatgactgacatttaaatcatgccttaaccaaaaacgttatatcggtattatattaagaagagaccaaaatatggtcctgtcgagaaaatttctaaacattagttctcatcaccagtgagccgtcaccatctagtttgcaacggtccagttagagtgcactcaggactcgcagcgagagaatttttttaatcaagcctaaaattcactttcggacaaatcgaactactcataaatattaaccatgagaccttttcgccgcagcaggttttctatcggccgttagattttagtgacgatgaaaatgatagaacgcaacgtgccgcatgcatccattcccattcgttttccacagtacatgtaggagtactgtgcaagtagggtccgtacattcagtctctctcactagttggactcttctactgctacaaagacatgagctgccgggaatgggaaccggaggagcgagcgagcctgacggtctcacacacacagtcacactcccaagccaattattataagaggcgagatgagcaactccagctcttaaccaatccactcctcctccctctccacctcctctgctttgctctgccactctgctgaggtggggggcagaggagctccccctccctcctctcccctcctcgccatgtctagcagcgacccggaggagatcagggcgcgcgtcgtcgttctcggttcgccccatgccgacggcggcgacgagtgggcccggcccgagctcgaggccttccatctgccgtctcccgcccaccagcctcctggcttcctagccgggcaaccggaagcagcagagcaacccacgctccctgctcctgctggccgcagcagcagcagcagcaacacgcctactacatctgccggtggcggcgctgctcctcctcctccttcttcgcctccccctccgccggcttctctggagaccgagcagccgcccaatgccaggccagcctccgccggcgccaatgacagcaagaagcccaccccgcccgccgccctgcgcgacctcttccgcttcgccgacggcctcgactgcgcgctcatgctcatcggcaccctcggcgcgctcgtccacggctgctcgctccccgtcttcctccgcttcttcgccgacctcgtcgactccttcggctcccacgccgacgacccggacaccatggtccgcctcgtcgtcaagtacgccttctacttcctcgtcgtcggagcggcaatctgggcatcctcgtgggcaggtacgctatccctcctcctcctgccgccccagcttgtgtgcgtcgcgaattggcggtcaatttggattggatgacaaatcacgtcggtcagccaatcgccgtggctacaaacgagatgttcaaatcgttcgccccgctcgcagagatctcttgctggatgtggaccggcgagcggcagtcgacgcggatgcggatccggtacctggacgcggcgctgcggcaggacgtgtccttcttcgacaccgacgtgcgggcctcggacgtgatctacgccatcaacgcggacgccgtggtggtgcaggacgccatcagcgagaagctgggcaacctcatccactacatggccaccttcgtggccggcttcgtcgtggggttcacggccgcgtggcagctggcgctggtcacgctggccgtggtgccgctcatcgccgtcatcggcgggctgagcgccgccgcgctcgccaagctctcgtcccgcagccaggacgcgctctcgggcgccagcggcatcgcggagcaggcgctcgcgcagatacggatcgtgcaggcgttcgttggcgaggagcgcgagatgcgggcctactcggcggcgctggccgtggcgcagaggatcggctaccgcagcggcttcgccaaggggctcggcctcggcggcacctacttcaccgtcttctgctgctacgggctcctgctctggtacggcggccacctcgtgcgcgcccagcacaccaacggcgggctcgccatcgccaccatgttctccgtcatgatcggcggactgtaaggcccaccacaccacgcactctctccttctgctgctcctcggcccgcccccgtcgtcattgctgctgacggtatctgtggatcgcgtgcagggccctcgggcagtcggcgccgagcatggccgcgttcgccaaggcgcgtgtggcggctgccaagatcttccgcatcatcgaccacaggccgggcatctcctcgcgcgacggcgcggagccagagtcggtgacggggcgggtggagatgcggggcgtggacttcgcgtacccgtcgcggccggacgtccccatcctgcgcggcttctcgctgagcgtgcccgccgggaagaccatcgcgctggtgggcagctccggctccgggaagagcacggtggtgtcgctcatcgagagattctacgaccccagcgcaggtatacctagtactgttactacttttagcgcattaatctgaggatgtccagttcgcttgcttgccaatcgccattgccatcgcaacaacaatacttcgccaactgccattgctgggtagactagtacagtagcagttagaagaagcctccactgtacattgcattgccaaacaaaagtgaattgtgcagtaactctgtaccaccacattgacatggaaatgaagtgaatgcttggagcatgcagagctggccggcctcatgggctgctgctacctgctagctagccaaccagaaccagccatcctctttcttgcttttctttttactttctttggtcgtggctgtttgtggtcatacatacattcacgcagagcagaagagctagctaagctaggtgggtgtgcctgcaacgcgggacaaagaaaactatttgttgcctggcaagatgctactgttgcctagcacatgcctgccattgaccgactgctcagtgagaagtggttcagttgtgctgttgacagtatagatagatatatatagtagccctgtagatttttttttcagacaaaaaaagaagaagaacgagatgaagtctgcaattcggttttggcagggcaaatcctgctggacgggcacgacctcaggtcgctggagctgcggtggctgcggcggcagatcgggctggtgagccaggagccggcgctgttcgcgacgagcatcagggagaacctgctgctggggcgggacagccagagcgcgacgctggcggagatggaggaggcggccagggtggccaacgcccactccttcatcatcaaactccccgacggctacgacacgcaggtccgtcccgtatagctagctcactagctgcactgccacttctctcgcttgctccccaccgttgctgcctgttgctctccaatccacttgtcggtgtctggaccacacgtgcctgcttgcctagctgctccacatctgctttccctgtccaaccttatgcaactcactctaatactatatcaaatacatttctagagtttaaagcttatcttagaataaatgcatctttagctacgagacaacctaacttcagttgttgttgttgttttttttactttctctcttctcacaaatactatgattacgtctttacagcgatcttttttattccaaacctaaaaatgcatgcactcactctaaaagcgcaaagggagcatctttttttcccccatcatctgcacgcagccttttcttttcctcatgtcacgagggactgaaggtgtgtatgcagcgtcaagtcatccatccgttccactccactcactcatgcgtcgcgcactctgcgctcgtgcctgcccggggctaaagctttagtagctagcctcagatcagatactgttcgtgtttgttaggccgcggcagctgcacatgagctcatgacagccggcagcaccaccaccaacgccatggaagaggggtcggggtccatcacatagacatagatgcctgttgtagactaggacgggagggcaattgttaggcgcctgttgccatcgcatttgctgctgtgggttgccaacaagtaacatgccaggatgctttgctatcacgcacaggacaggagaggtcctttttctcgacacaagctctacagcctctactaaactagcacttgctgatgagtgcagaggatgaatggacgatgaacatctagagtgagagagaaaaaaatgttaataataataaaaagtagtagcaggattaagaatcaacctggggtacgtaggaagaggtacaatccctaggaatctagagtatgagaagtatgggaggagttgggggagtgaaacggaacaaattccgagttggtattttgtcgggaatgtcaagttgatttttgatcctagtgcaagcaagaattatcaatcactcagactcagcctgtctgtgtctgtccaccccagctcttgctactctacttactactgtgctactagtggtagggtaggtatcttacataaactgttattataaactgtcatctgagaaagagagccagtcaaacccatgctgctgcttattttaatcactgtcaaatggcaggcaggcaggcagtctggttagttaataacatctgggaagggtttaatcaaaccaaatcaaatcagacgaaatctagaggccacatgggatggggccatatgtactgtactagcataactagcggctagattttattagaacacggactcacactcccataactataactgacttgatcatgattccttgccaagcaatgctcgcatgcccatgcatgcatcatccctggtcaaactcaaacactctccaccgtcagggaataagacttattattttattaacaattcaatttttatttattaattacgtctggacgaggagtactggtttatttgatgagagacatggcagtccaagtcaaactcgtttgtctgaccatggcggtgatggccggtgcaggttggggagcgcggcctgcagctctccggtgggcagaagcagcgcatcgccatcgcccgcgccatgctcaagaaccccgccatcctgctgctggacgaggccaccagcgcgctggactccgagtctgagaagctcgtgcaggaggcgctggaccgcttcatgatcgggcgcaccaccctggtgatcgcgcacaggctgtccaccatccgcaaggccgacgtggtggccgtgctgcagggcggcgccgtctccgagatgggcgcgcacgacgagctgatggccaagggcgagaacggcacctacgccaagctcatccgcatgcaggagcaggcgcacgaggcggcgctcgtcaacgcccgccgcagcagcgccaggccctccagcgcccgcaactccgtcagctcgcccatcatgacgcgcaactcctcctacggccgctccccctactcccgccgcctctccgacttctccacctccgacttcaccctctccatccacgacccgcaccaccaccaccggaccatggcggacaagcagctggcgttccgcgccggcgccagctccttcctgcgcctcgccaggatgaactcgcccgagtgggcctacgcgctcgccggctccatcggctccatggtctgcggctccttcagcgccatcttcgcctacatcctcagcgccgtgctcagcgtctactacgcgccggacccgcggtacatgaagcgcgagatcgcaaaatactgctacctgctcatcggcatgtcctccgcggcgctgctgttcaacacggtgcagcacgtgttctgggacacggtgggcgagaacctgaccaagcgggtgcgcgagaagatgttcgccgccgtgctccgcaacgagatcgcctggttcgacgcggacgagaacgccagcgcgcgcgtggccgccaggctagcgctggacgcccagaacgtgcgctccgccatcggggaccgcatctccgtcatcgtccagaactcggcgctgatgctggtggcctgcaccgcggggttcgtcctccagtggcgcctcgcgctcgtgctcctcgccgtgttcccgctcgtcgtgggcgccaccgtgctgcagaagatgttcatgaagggcttctcgggggacctggaggccgcgcacgccagggccacgcagatcgcgggcgaggccgtggccaacctgcgcaccgtggccgcgttcaacgcggagcgcaagatcacggggctgttcgaggccaacctgcgcggcccgctccggcgctgcttctggaaggggcagatcgccggcagcggctacggcgtggcgcagttcctgctgtacgcgtcctacgcgctggggctgtggtacgcggcgtggctggtgaagcacggcgtgtccgacttctcgcgcaccatccgcgtgttcatggtgctgatggtgtccgcgaacggcgccgccgagacgctgacgctggcgccggacttcatcaagggcgggcgcgcgatgcggtcggtgttcgagacgatcgaccgcaagacggaggtggagcccgacgacgtggacgcggcgccggtgccggagcggccgaggggcgaggtggagctgaagcacgtggacttctcgtacccgtcgcggccggacatccaggtgttccgcgacctgagcctccgtgcgcgcgccgggaagacgctggcgctggtggggccgagcgggtgcggcaagagctcggtgctggctctggtgcagcggttctacgagcccacgtccgggcgcgtgctcctggacggcaaggacgtgcgcaagtacaacctgcgggcgctgcggcgcgtggtggcggtggtaccgcaggagccgttcctgttcgcggcgagcatccacgagaacatcgcgtacgggcgcgagggcgcgacggaggcggaggtggtggaggcggcggcgcaggcgaacgcgcaccggttcatcgcggcgctgccggaggggtaccggacgcaggtgggcgagcgcggggtgcagctgtcgggggggcagcggcagcggatcgcgatcgcgcgcgcgctggtgaagcaggcggccatcgtgctgctggacgaggcgaccagcgcgctggacgccgagtcggagcggtgcgtgcaggaggcgctggagcgcgcggggtccgggcgcaccaccatcgtggtggcgcaccggctggccacggtgcgcggcgcgcacaccatcgcggtcatcgacgacggcaaggtggcggagcaggggtcgcactcgcacctgctcaagcaccatcccgacgggtgctacgcgcggatgctgcagctgcagcggctgacgggcgcggcggccgggcccgggccgtcgacctcgtgcaacggggccgcgtaggacggaatggatggatggatgggtttggttcctcgagagattgatgggtgaggaagctgaagctccggatcaaatggtggtactccatgatcgcaacaatgaggggaaaaaaagaaaggagaaaatacggtggttcatatgattgtacaatttgacgatctgtttgagtcggggttttaggatgatgtaaaccttcactcgccttttttttactcttgtttctcatccgcatcagtatcatctatctacatacagtgtcagagatgggaactgatccgcatcatcatctacctcccaaggcaccccagattgtattaatgtacttagttagcctgttttatatatacttataagtaccaaatagcagaattttactccttatctgcagtagcacgaaagaagaaacggcaagctacacctatctctggaagcaaaagaatctccccattctctctcttcctacgaggacaccccatccttttcagacagacagacagacggaccaaactgcaccacccaagaaccgatcgagagcagaggtagcattctctgcactgtagagtgtagaccatactacgctgctctcgagacatgatcccgggaatatctaccgaattgtttgtcagtggcatagccgcatagctctcagctgcctaatgaattgttt

SEQ ID NO: 45 - DNA, 4786 bp, Zea mays
  source 1..4786: mol_type = unassigned DNA
gcaagtagggtccgtacattcagtctctctcactagttggactcttctactgctacaaagacatgagctgccgggaatgggaaccggaggagcgagcgagcctgacggtctcacacacacagtcacactcccaagccaattattataagaggcgagatgagcaactccagctcttaaccaatccactcctcctccctctccacctcctctgctttgctctgccactctgctgaggtggggggcagaggagctccccctccctcctctcccctcctcgccatgtctagcagcgacccggaggagatcagggcgcgcgtcgtcgttctcggttcgccccatgccgacggcggcgacgagtgggcccggcccgagctcgaggccttccatctgccgtctcccgcccaccagcctcctggcttcctagccgggcaaccggaagcagcagagcaacccacgctccctgctcctgctggccgcagcagcagcagcagcaacacgcctactacatctgccggtggcggcgctgctcctcctcctccttcttcgcctccccctccgccggcttctctggagaccgagcagccgcccaatgccaggccagcctccgccggcgccaatgacagcaagaagcccaccccgcccgccgccctgcgcgacctcttccgcttcgccgacggcctcgactgcgcgctcatgctcatcggcaccctcggcgcgctcgtccacggctgctcgctccccgtcttcctccgcttcttcgccgacctcgtcgactccttcggctcccacgccgacgacccggacaccatggtccgcctcgtcgtcaagtacgccttctacttcctcgtcgtcggagcggcaatctgggcatcctcgtgggcagagatctcttgctggatgtggaccggcgagcggcagtcgacgcggatgcggatccggtacctggacgcggcgctgcggcaggacgtgtccttcttcgacaccgacgtgcgggcctcggacgtgatctacgccatcaacgcggacgccgtggtggtgcaggacgccatcagcgagaagctgggcaacctcatccactacatggccaccttcgtggccggcttcgtcgtggggttcacggccgcgtggcagctggcgctggtcacgctggccgtggtgccgctcatcgccgtcatcggcgggctgagcgccgccgcgctcgccaagctctcgtcccgcagccaggacgcgctctcgggcgccagcggcatcgcggagcaggcgctcgcgcagatacggatcgtgcaggcgttcgttggcgaggagcgcgagatgcgggcctactcggcggcgctggccgtggcgcagaggatcggctaccgcagcggcttcgccaaggggctcggcctcggcggcacctacttcaccgtcttctgctgctacgggctcctgctctggtacggcggccacctcgtgcgcgcccagcacaccaacggcgggctcgccatcgccaccatgttctccgtcatgatcggcggactggccctcgggcagtcggcgccgagcatggccgcgttcgccaaggcgcgtgtggcggctgccaagatcttccgcatcatcgaccacaggccgggcatctcctcgcgcgacggcgcggagccagagtcggtgacggggcgggtggagatgcggggcgtggacttcgcgtacccgtcgcggccggacgtccccatcctgcgcggcttctcgctgagcgtgcccgccgggaagaccatcgcgctggtgggcagctccggctccgggaagagcacggtggtgtcgctcatcgagagattctacgaccccagcgcagggcaaatcctgctggacgggcacgacctcaggtcgctggagctgcggtggctgcggcggcagatcgggctggtgagccaggagccggcgctgttcgcgacgagcatcagggagaacctgctgctggggcgggacagccagagcgcgacgctggcggagatggaggaggcggccagggtggccaacgcccactccttcatcatcaaactccccgacggctacgacacgcaggttggggagcgcggcctgcagctctccggtgggcagaagcagcgcatcgccatcgcccgcgccatgctcaagaaccccgccatcctgctgctggacgaggccaccagcgcgctggactccgagtctgagaagctcgtgcaggaggcgctggaccgcttcatgatcgggcgcaccaccctggtgatcgcgcacaggctgtccaccatccgcaaggccgacgtggtggccgtgctgcagggcggcgccgtctccgagatgggcgcgcacgacgagctgatggccaagggcgagaacggcacctacgccaagctcatccgcatgcaggagcaggcgcacgaggcggcgctcgtcaacgcccgccgcagcagcgccaggccctccagcgcccgcaactccgtcagctcgcccatcatgacgcgcaactcctcctacggccgctccccctactcccgccgcctctccgacttctccacctccgacttcaccctctccatccacgacccgcaccaccaccaccggaccatggcggacaagcagctggcgttccgcgccggcgccagctccttcctgcgcctcgccaggatgaactcgcccgagtgggcctacgcgctcgccggctccatcggctccatggtctgcggctccttcagcgccatcttcgcctacatcctcagcgccgtgctcagcgtctactacgcgccggacccgcggtacatgaagcgcgagatcgcaaaatactgctacctgctcatcggcatgtcctccgcggcgctgctgttcaacacggtgcagcacgtgttctgggacacggtgggcgagaacctgaccaagcgggtgcgcgagaagatgttcgccgccgtgctccgcaacgagatcgcctggttcgacgcggacgagaacgccagcgcgcgcgtggccgccaggctagcgctggacgcccagaacgtgcgctccgccatcggggaccgcatctccgtcatcgtccagaactcggcgctgatgctggtggcctgcaccgcggggttcgtcctccagtggcgcctcgcgctcgtgctcctcgccgtgttcccgctcgtcgtgggcgccaccgtgctgcagaagatgttcatgaagggcttctcgggggacctggaggccgcgcacgccagggccacgcagatcgcgggcgaggccgtggccaacctgcgcaccgtggccgcgttcaacgcggagcgcaagatcacggggctgttcgaggccaacctgcgcggcccgctccggcgctgcttctggaaggggcagatcgccggcagcggctacggcgtggcgcagttcctgctgtacgcgtcctacgcgctggggctgtggtacgcggcgtggctggtgaagcacggcgtgtccgacttctcgcgcaccatccgcgtgttcatggtgctgatggtgtccgcgaacggcgccgccgagacgctgacgctggcgccggacttcatcaagggcgggcgcgcgatgcggtcggtgttcgagacgatcgaccgcaagacggaggtggagcccgacgacgtggacgcggcgccggtgccggagcggccgaggggcgaggtggagctgaagcacgtggacttctcgtacccgtcgcggccggacatccaggtgttccgcgacctgagcctccgtgcgcgcgccgggaagacgctggcgctggtggggccgagcgggtgcggcaagagctcggtgctggctctggtgcagcggttctacgagcccacgtccgggcgcgtgctcctggacggcaaggacgtgcgcaagtacaacctgcgggcgctgcggcgcgtggtggcggtggtaccgcaggagccgttcctgttcgcggcgagcatccacgagaacatcgcgtacgggcgcgagggcgcgacggaggcggaggtggtggaggcggcggcgcaggcgaacgcgcaccggttcatcgcggcgctgccggaggggtaccggacgcaggtgggcgagcgcggggtgcagctgtcgggggggcagcggcagcggatcgcgatcgcgcgcgcgctggtgaagcaggcggccatcgtgctgctggacgaggcgaccagcgcgctggacgccgagtcggagcggtgcgtgcaggaggcgctggagcgcgcggggtccgggcgcaccaccatcgtggtggcgcaccggctggccacggtgcgcggcgcgcacaccatcgcggtcatcgacgacggcaaggtggcggagcaggggtcgcactcgcacctgctcaagcaccatcccgacgggtgctacgcgcggatgctgcagctgcagcggctgacgggcgcggcggccgggcccgggccgtcgacctcgtgcaacggggccgcgtaggacggaatggatggatggatgggtttggttcctcgagagattgatgggtgaggaagctgaagctccggatcaaatggtggtactccatgatcgcaacaatgaggggaaaaaaagaaaggagaaaatacggtggttcatatgattgtacaatttgacgatctgtttgagtcggggttttaggatgatgtaaaccttcactcgccttttttttactcttgtttctcatccgcatcagtatcatctatctacatacagtgtcagagatgggaactgatccgcatcatcatctacctcccaaggcaccccagattgtattaatgtacttagttagcctgttttatatatacttataagtaccaaatagcag

SEQ ID NO: 46 - DNA, 20 bp, Zea mays
  source 1..20: mol_type = unassigned DNA
acgtgcgcaagtacaacctg

SEQ ID NO: 47 - DNA, 20 bp, Zea mays
  source 1..20: mol_type = unassigned DNA
agtacaacctgcgggcgctg

SEQ ID NO: 48 - DNA, 20 bp, Zea mays
  source 1..20: mol_type = unassigned DNA
cgtgcgcaagtacaacctgc

SEQ ID NO: 49 - DNA, 20 bp, Zea mays
  source 1..20: mol_type = unassigned DNA
tgctggacgggcacgacctc

SEQ ID NO: 50 - DNA, 18 bp, Zea mays
  source 1..18: mol_type = unassigned DNA
cgccatgctcaagaaccc

SEQ ID NO: 51 - DNA, 20 bp, Zea mays
  source 1..20: mol_type = unassigned DNA
ggttcgacgcggacgagaac

SEQ ID NO: 52 - DNA, 20 bp, Zea mays
  source 1..20: mol_type = unassigned DNA
ggtgttccgcgacctgagcc

SEQ ID NO: 53 - DNA, 20 bp, Zea mays
  source 1..20: mol_type = unassigned DNA
gaacgcgcaccggttcatcg

SEQ ID NO: 54 - DNA, 20 bp, Zea mays
  source 1..20: mol_type = unassigned DNA
gttggactcttctactgcta

SEQ ID NO: 55 - DNA, 20 bp, Zea mays
  source 1..20: mol_type = unassigned DNA
tgccactctgctgaggtggg

SEQ ID NO: 56 - DNA, 20 bp, Zea mays
  source 1..20: mol_type = unassigned DNA
gtatcgcgagatgcttattt

SEQ ID NO: 57 - DNA, 20 bp, Zea mays
  source 1..20: mol_type = unassigned DNA
agcagcattaaccgagtgaa

SEQ ID NO: 58 - DNA, 20 bp, Zea mays
  source 1..20: mol_type = unassigned DNA
cagagtgcaggacataactc

SEQ ID NO: 59 - DNA, 20 bp, Zea mays
  source 1..20: mol_type = unassigned DNA
ggacaaattgaacctggaac

SEQ ID NO: 60 - DNA, 20 bp, Zea mays
  source 1..20: mol_type = unassigned DNA
catgcatccattcccattcg

SEQ ID NO: 61 - DNA, 20 bp, Zea mays
  source 1..20: mol_type = unassigned DNA
catgcatccattcccattcg

SEQ ID NO: 62 - DNA, 21 bp, Zea mays
  source 1..21: mol_type = unassigned DNA
ttattagctggctagctaggc

SEQ ID NO: 63 - DNA, 22 bp, Zea mays
  source 1..22: mol_type = unassigned DNA
tccacggactcggcgcgggagc

SEQ ID NO: 64 - DNA, 22 bp, Zea mays
  source 1..22: mol_type = unassigned DNA
cctacttcggcgaggcgcttgc

SEQ ID NO: 65 - DNA, 17 bp, Zea mays
  source 1..17: mol_type = unassigned DNA
cgtgtatcgcttccgcc

SEQ ID NO: 66 - DNA, 17 bp, Zea mays
  source 1..17: mol_type = unassigned DNA
ctacctgaagttcgccc

SEQ ID NO: 67 - DNA, 25 bp, Zea mays
  source 1..25: mol_type = unassigned DNA
gttcgcgcacaccatccgcgtggac

SEQ ID NO: 68 - DNA, 21 bp, Zea mays
  source 1..21: mol_type = unassigned DNA
gagggcgatgacacggatgac

SEQ ID NO: 69 - DNA, 19 bp, Zea mays
  source 1..19: mol_type = unassigned DNA
ggtcatgtcggaggtgtac

SEQ ID NO: 70 - DNA, 1149 bp, Zea mays
  source 1..1149: mol_type = unassigned DNA
atgccgtgctcgtcgccggccccgacatggctgctgcgagtgtcgccagcggctgccgcggccgaccaggccgccgcctcgtcctcgtgctcatccaagggcggaggccgcgtgctcacggccggtaccaccaccatggacacggccgccaccgctgccgccggcggcaatgccgccgacctccaggagagcagcagcagcggccagtcccggctcgcggcgcgcggccactggcgcccagcggaggacgccaagctccgtgagctcgtcgcgctgtacgggccccagaactggaacctcatcgccgagaagctggacggcagatccgggaagagctgccgcctccgctggttcaaccagctagacccccggatcagcaagcgccccttcagcgacgaggaggaggagcgcctgatggctgcgcaccgcttctacggcaacaagtgggccatgatcgcgcgcctcttccccggccgcacggacaacgccgtgaagaaccactggcacgtcatcatggcgcgcaagtaccgcgagcagtccacggcctaccgccgccgcaagctcaaccaggcagtccagcggaagctcgaggcagcctccgccgcggtcgcaatgccgccgggcgcgggcgcgggagacgtcgccgtcggccagcaccaccacctgctggccgccgccgcggcggcccacgcccacgacgccgcctacagcttcgccgcggacccctacggcttcggcatccgccaccaatactgcaccttcccgttcccgccaggcgccgcttcggctgaggacccgccgccgccaacgcaaatacatcccttctgcttgttccctgggcccagcagcgcggcggcgcacgccgacagcaggcgccttccctggccgccgtcgtcggacgcgcccggcgtcgcccggtacggggagccgcatcagctcctgcagctgcccgttcaaagcggctggatcgacggcgtcggcgtggccgcggccggccaccacgagccgcccttcgtcttgggcaacaacgggggcgcggccgcctttgaagggacgacaagacagcagggctccggcgctcactttgaagctgccgcggcgccgccgccgccagcgttcatagatttcctcggggtcggagccacatga

SEQ ID NO: 71 - DNA, 6180 bp, Zea mays
  source 1..6180: mol_type = unassigned DNA
ttgttttacctaaggtttcttcatttctctgttttcccgtatgtgtagggccttcccctccctcccctcacctcccccggcggcggcgcccctcacccctccttttttccccaccggtgcgaccccctctccccccggcagtcagcggcggcggcggaggcgcccgcgcccagcccgcagccctgcacgcgcgctcctatgcccctgcctgcgcgcccccccccctgcgcagccccctccccctcctcctctctcttctatggatgggaggaagaagatgagatgagtagaaagatggcggttttgtaatttagtacgcatgaatttcagtttagtttaaaacagtgatagcttttacgttttaaacccgatttaatccttttaagttgtgttagatttatattcaaattatctacatgttagaagtattttccctaatattgtatattttatttaatttatttaaacatttgtttgatcgatgactaatagaacgacgttttatttaaatctaatttataatttcgaattgcacatttgtaaatcaacatcagcgcagtcaatgttaactgaactattctattaaataattagttagtttaatcatattatttgtttattagtgttcgcgtatggtgttggcctgcgcgccgtgcgcgtaccgtgcgcgtgcggccgtactatttcacgctttacgtattacgtcttacaccactaattcgtcttgcttagagtcgctaatgttatttaaagtaattatttaactaagagttgttagtgcaatacaactaattaaaactaggcgatagctctagttgcatttaacaaatagcgattaatatagttacgtcgaattaaatattctaattaatacttacttagtgtaaacacgttaacccctcgaccgtagcttcgactattgcggttctttttctcgcgtaaccgtagaagtgctctctattttatattgctcgcttttataatattatatattgtatggtgtaatgttcttatgcaaatatattattgggggccttccccttccgaaggtcctaaaaacataattaaccatttggctttagcatgaactattacaggaagcttcgtctctaggagataagcctctttctaatgacgaaagacacacatgatgaagatagatctaaagaagacaagagtaaacgccgaagctaatagcggacataaatagctgaagaaggaaaacggaggaatgctgataatggctgaagaaggaaaagactatttggtcctttataatttgtattacgatcatgtgtaaacattaagaacataaatgaacttttgctcgggctgcgtcccgtgcctataaatagatgaacagtaacatcgtactgttcaggctgaattgtattcactctctcgcatcctcgccttcaacaagccgaaggtactaatgtaatattattattatagatattcatatatgttttatggaatgaaagaataaaagaattattatgatttaactatcatatttatttccttgtatccctatctttgtattgatatgatgaaggtgtgtccttcctgaccttcgtccgaagagtattacacccgtaaggagataatacttcgagggacgaaggtcctttacgattaacaattgtgttgccttgttcttgacttacaccatttgagaacaagtgaccaacatttggcgcccacctccggtgaactcacttccacaaccttcggcaagcatcaaccttcgacatgccgccgaagaaggcaacgatgccagccactggacacaaaccaggacgcactctccttgcgcgaggctaggaaccaaaagaggaaggccactagcccaactcttcaaaaggaccagcttgaccaggagatcagggatttagaagcaatccatcaacaggtgcaaagaaaaagtgagaaaatgctccggctggccgatcttcagaagaagattgacgacgcagctgaggagatgcgtcatcttactcaagatggccaagatcgaaggcctcagcacagggagcttcgtcaggagagctcattcaacgaagatgaatggtacaatgactttcatcatggtaactttacttttgatgatgcttctcctctggtggcagaattgcaggctaccccgtggccataatcttacaagccaccttagttgcccatgtatgatgggcactcggatccaaagcaatttctgatgagttacgaggcaacaatatcctcgtacgggggcaacgctactatcatggcaaagttcttcgtcatggcagtcagaagcgtggcctagacatggtattcctcccttagatcagggacaatcacatcatggcagaagctgaaggatatgctggtcactagtttccagggctttcagacaaagccaattattgctcaggccttgttccagtgcacgcaagaccaggaggagtacctgtaggcttacgtccgaaggttcctacgtttgagagctcaatcgcctatagtgcccaatgaaattgtcattgaggccatgattaaggggcttcggccaggacctacaaccaaatatttcgctaggaagcccccacaaaccctggagaagttccttaagaagatggatgagtacatctgggtcgataatgatttccgccaaagaagggaggaagcatacaagttttctgagatgaccaggggcttcggaggaggacttcatcccaggcatatcaggtcaatccataactccaatgctaacgatgaaaggcccaatagtgctcagagcggccatcatcgctcacagtcttcgagcatgcagcaaacttcctataggccaccagctccgagaggcagaggaggaagaagcttcagtggaggaagattcggtaatcaacccaggaagttgtattgcctcttctgtgacgaggttaagggccacacaacaaggacgtgtcaggtcacaatccagaagcaaaaggaaattgttgaagccgaggcatggcagaaccagccgaagcaagtcctttatactgcttcgtgctactctccatacatcccagaatatgtaggcaaccaatagactacagcttcaccaagtcactcccaagcttcctgggcccaattactgccacccccaccaatggtgcctgccccaagccatgataagcagccagaagggcacctttggcctcaacaacaacgtgatcttcgggatcagtctgaggttcgcacagttaacagtactgtacctgaggccaggcacatctactgaagatgacacatcgttttggtcaaaagaaagtcctgatatgtcccatttctactattttttgctttcatatttctgttgcaaaagacaatatagtaaggtttaacattcaacttgatgtaataaacctatcgttacaccatcgagtgtgacaaaatcaagttcctaagctccaaagacgttcctaagggagcgcagagtaagttctgaagctcaaaagtcgtttctaagggaatacagagccaaaatgccacctaagtaaaaggtgaagagactccaaatcactcctaagggaatgcagagcctgaatgccacctaagtaacaggtgaagaagttcaaaagtcgttcctaaggggatgcagagctgaaatactacctaagtaaagggtgaagagactccaaagtcgttcctaaggggacgcaaagtctggatgtgtattcgtgtaggttttttttaccttcggcataaatattattttgcatcataccatcataacatatcgcatagcattgtatcatacatcattttgcatcagcaaaaggctatggagaagaagggaaattgctccttcgcaacatgtatcttcggtggatataatttactacacgaagcccaccttcgtcaacatctttgagcaaactcaatgttttatactcgaacaaaatatattgagatagattttccattcttcgtgggaacgccaagctgattcgaggtgtttagatatttgatttattagttctgcggagcacaaaaggcttttgcctacatggtagtagatgatgttatttatgaacagcagccctcaactcaagtgatgcattatgatattattattattattatcgggagttttggagacacaataatgtcgctgggtgtggacgaacgaagccgtgcgtgcgtgcgtgcgccgggggcagagcggcagcggcacagtgcgcggggcctgcgcccccccgtgcagttgaaaagataggtgcctgtagtggttgcagggctcattcagacagcactggagggcatgcatgttctgtcaaggcatcagggctccgcgcctgggagatagatcatctcatctactctactactagtctactacccaggcaactcaaagcaatgcaagagagcctgtttattgttgggactcgtaccccgcccagcagctctgcacatttaacaatcccactcatgcttttgtttcttattattattattattattacaaaaaaaaaagaaaataggccgaacctgctgcctgaaacccgcatgcaccacctgcaggggcctctatttaatttgcgctggtctcgtacttgacgttgcgtcctgctgctcctttgagattcctgctggaagattgcgatctctgccttcttttcttttcttctttttttttaaaaaaaacaaaaggcatcagtttgagtacttttattggctaagtacgaaaacattaactcccggtcaagagaaaggtggtgtgtgtttgtgcgtgcgtgcgtgtgtgtttaataaggcccagcagcctccctgagctggtcgttttatatggccagtcaagcgttgcagagtagtatattgtctatgcattactacttgactaagcagccactgaactctgcacaggtctacttggccctcagagtacacattattcaccggccagtcgtcgtgcaaagagcacagtttctgttaccgctgatgattggatgccgtaattaatagccggttcccattcccgtccagatttccatcgcgattcgcgaggaaaagcgtccgtgtgtgtgtacacgcgcttcaaactgttgggcgcatgtacacgtacgtacgctcggacgtaccgatccactgttgagagtgaaaactgaatggggagggggagagagagaagaaagagaaagagaccagtagcaagtagtagtagatcgacgatcaggcaggcgggctacagtgctaaccttctctctctctatctctctctggccgtgcgtcgtccttgcaagccacacatgtggtgagatgacatctacacgtgcggtggggacgcccgatgctttgttaaactcggtgaaagccatgagagatggcagcgggcggtgtggtggtagtagtagacagctgggacagtgacaagccttgcgtgcagtaagatcttctgcgcccttaccttaattacccctcctccgcctccagtttttaccgcgcgaaccctagtaaaataactcccgccagtgtgctctctctctctctctacgactatttttcaccccttctttcccagttccgtgccttgcacttcgccttttcaaaaagcttttgccatatgcagtacacatgtgttaatagagtagtaactttcttttcttttgcaaactgattgagatccaaagcaagcaagcaagcaagctgtatgtacttgcaaacaacacccttggtagctatccccctcccgtgcccgtgcccgtgcccgtgccctgcaatccccgacccggagcagcagccaccaccggcgcggcgtcccgcgagccagcacagacgatccctccccattcccgcccgcactgcccagcaccaggaggagcagctagcctatccaacagtgaaaagcacacacgcgttccggactccggactacgcccggccctcccctcccctccccctcctcctgcggtttttagacaggggagtgcgtgcgcccgagcgatccgtccatctgacgggaatgagagggtgcgtgcgtgcgtgggggagagtgagatgcctgcctccctgtagcgtgtaggagtagctctggcctcttcctctacctccagccgtgcggttttctgctgcggaagaaacgggagcagtgtcgctcgtcccgctcgcgcgcacatcctcaactcgtctccgtctctcccgcggcaactgacgacg

SEQ ID NO: 72 - DNA, 939 bp, Zea mays
  source 1..939: mol_type = unassigned DNA
agatctagagattcgtcttcgtgcgattaatctcgttttttttccgtgtggatgctttgttttgaaaagactgaatgaattgtgtgcgtcaaggatcggagagggattcagaacataagtgatggaccctctttcttagatgattatgtatccagtctttatcaatgcatgatgctgaagcgacaaaacctagacatatgtctttcaggtgaggctctgcacagatctgaaactgagttactgttcatctttgatgttgagagacagacgaactctccacatctgatgcaatgactgacatttaaatcatgccttaaccaaaaacgttatatcggtattatattaagaagagaccaaaatatggtcctgtcgagaaaatttctaaacattagttctcatcaccagtgagccgtcaccatctagtttgcaacggtccagttagagtgcactcaggactcgcagcgagagaatttttttaatcaagcctaaaattcactttcggacaaatcgaactactcataaatattaaccatgagaccttttcgccgcagcaggttttctatcggccgttagattttagtgacgatgaaaatgatagaacgcaacgtgccgcatgcatccattcccattcgttttccacagtacatgtaggagtactgtgcaagtagggtccgtacattcagtctctctcactagttggactcttctactgctacaaagacatgagctgccgggaatgggaaccggaggagcgagcgagcctgacggtctcacacacacagtcacactcccaagccaattattataagaggcgagatgagcaactccagctcttaaccaatccactcctcctccctctccacctcctctgctttgctctgccactctgctgaggtggggggcagaggagctccccctccctcctctcccctcctcgcc

SEQ ID NO: 73 - DNA, 1893 bp, Zea mays
  source 1..1893: mol_type = unassigned DNA
atgaagcgcgagtaccaagacgccggcgggagtggcggcgacatgggctcctccaaggacaagatgatggcggcggcggcgggagcaggggaacaggaggaggaggacgtggatgagctgctggccgcgctcgggtacaaggtgcgttcgtcggatatggcggacgtcgcgcagaagctggagcagctcgagatggccatggggatgggcggcgtgggcggcgccggcgctaccgctgatgacgggttcgtgtcgcacctcgccacggacaccgtgcactacaatccctccgacctgtcgtcctgggtcgagagcatgctgtccgagctcaacgcgcccccagcgccgctcccgcccgcgacgccggccccaaggctcgcgtccacatcgtccaccgtcacaagtggcgccgccgccggtgctggctacttcgatctcccgcccgccgtggactcgtccagcagtacctacgctctgaagccgatcccctcgccggtggcggcgccgtcggccgacccgtccacggactcggcgcgggagcccaagcggatgaggactggcggcggcagcacgtcgtcctcctcttcctcgtcgtcatccatggatggcggtcgcactaggagctccgtggtcgaagctgcgccgccggcgacgcaagcatccgcggcggccaacgggcccgcggtgccggtggtggtggtggacacgcaggaggccgggatccggctcgtgcacgcgctgctggcgtgcgcggaggccgtgcagcaggagaacttctctgcggcggaggcgctggtcaagcagatccccatgctggcctcgtcgcagggcggtgccatgcgcaaggtcgccgcctacttcggcgaggcgcttgcccgccgcgtgtatcgcttccgcccgccaccggacagctccctcctcgacgccgccttcgccgacctcttgcacgcgcacttctacgagtcctgcccctacctgaagttcgcccacttcaccgcgaaccaggccatcctcgaggccttcgccggctgccgccgcgtccacgtcgtcgacttcggcatcaagcaggggatgcagtggccggctcttctccaggccctcgccctccgccctggcggccccccgtcgttccggctcaccggcgtcgggccgccgcagcccgacgagaccgacgccttgcagcaggtgggctggaaacttgcccagttcgcgcacaccatccgcgtggacttccagtaccgtggcctcgtcgcggccacgctcgccgacctggagccgttcatgctgcaaccggagggcgatgacacggatgacgagcccgaggtgatcgccgtgaactccgtgttcgagctgcaccggcttcttgcgcagcccggtgccctcgagaaggtcctgggcacggtgcgcgcggtgcggccgaggatcgtgaccgtggtcgagcaggaggccaaccacaactccggcacgttcctcgaccgcttcaccgagtcgctgcactactactccaccatgttcgattctctcgagggcgccggcgccggctccggccagtccaccgacgcctccccggccgcggccggcggcacggaccaggtcatgtcggaggtgtacctcggccggcagatctgcaacgtggtggcgtgcgagggcgcggagcgcacggagcgccacgagacgctgggccagtggcgcagccgcctcggcggctccgggttcgcgcccgtgcacctgggctccaatgcctacaagcaggcgagcacgctgctggcgctcttcgccggcggcgacgggtacagggtggaggagaaggacgggtgcctgaccctggggtggcatacgcgcccgctcatcgccacctcggcgtggcgcgtcgccgccgccgccgctccgtga

SEQ ID NO: 74 - DNA, 2850 bp, Zea mays
  source 1..2850: mol_type = unassigned DNA
cgcagctccccacttctcatcgccccctttttttaatttgtggccatctttggggtggtgggcggaggatttctaactggatggtgaagtttgtctggcgaaaaggacggctgcgacgaacccgtccatcgatccaacgctgtgcgcgcgttgggggagggacctgccaggccccacctgcagcgacagactattgatagatgccttcctctctgatcacctgatggctgatgccttcgcggccgtcttcgcctgccgctgctactactagttgccttcctcgcttccccgtctcgccccagccgcttcccccctcccctaccctttccttccccactcgcacttcccaaccctggatccaaatcccaagctatcccagaaccgaaaccgaggcgcgcaagccattattagctggctagctaggcctgtagctccgaaatcatgaagcgcgagtaccaagacgccggcgggagtggcggcgacatgggctcctccaaggacaagatgatggcggcggcggcgggagcaggggaacaggaggaggaggacgtggatgagctgctggccgcgctcgggtacaaggtgcgttcgtcggatatggcggacgtcgcgcagaagctggagcagctcgagatggccatggggatgggcggcgtgggcggcgccggcgctaccgctgatgacgggttcgtgtcgcacctcgccacggacaccgtgcactacaatccctccgacctgtcgtcctgggtcgagagcatgctgtccgagctcaacgcgcccccagcgccgctcccgcccgcgacgccggccccaaggctcgcgtccacatcgtccaccgtcacaagtggcgccgccgccggtgctggctacttcgatctcccgcccgccgtggactcgtccagcagtacctacgctctgaagccgatcccctcgccggtggcggcgccgtcggccgacccgtccacggactcggcgcgggagcccaagcggatgaggactggcggcggcagcacgtcgtcctcctcttcctcgtcgtcatccatggatggcggtcgcactaggagctccgtggtcgaagctgcgccgccggcgacgcaagcatccgcggcggccaacgggcccgcggtgccggtggtggtggtggacacgcaggaggccgggatccggctcgtgcacgcgctgctggcgtgcgcggaggccgtgcagcaggagaacttctctgcggcggaggcgctggtcaagcagatccccatgctggcctcgtcgcagggcggtgccatgcgcaaggtcgccgcctacttcggcgaggcgcttgcccgccgcgtgtatcgcttccgcccgccaccggacagctccctcctcgacgccgccttcgccgacctcttgcacgcgcacttctacgagtcctgcccctacctgaagttcgcccacttcaccgcgaaccaggccatcctcgaggccttcgccggctgccgccgcgtccacgtcgtcgacttcggcatcaagcaggggatgcagtggccggctcttctccaggccctcgccctccgccctggcggccccccgtcgttccggctcaccggcgtcgggccgccgcagcccgacgagaccgacgccttgcagcaggtgggctggaaacttgcccagttcgcgcacaccatccgcgtggacttccagtaccgtggcctcgtcgcggccacgctcgccgacctggagccgttcatgctgcaaccggagggcgatgacacggatgacgagcccgaggtgatcgccgtgaactccgtgttcgagctgcaccggcttcttgcgcagcccggtgccctcgagaaggtcctgggcacggtgcgcgcggtgcggccgaggatcgtgaccgtggtcgagcaggaggccaaccacaactccggcacgttcctcgaccgcttcaccgagtcgctgcactactactccaccatgttcgattctctcgagggcgccggcgccggctccggccagtccaccgacgcctccccggccgcggccggcggcacggaccaggtcatgtcggaggtgtacctcggccggcagatctgcaacgtggtggcgtgcgagggcgcggagcgcacggagcgccacgagacgctgggccagtggcgcagccgcctcggcggctccgggttcgcgcccgtgcacctgggctccaatgcctacaagcaggcgagcacgctgctggcgctcttcgccggcggcgacgggtacagggtggaggagaaggacgggtgcctgaccctggggtggcatacgcgcccgctcatcgccacctcggcgtggcgcgtcgccgccgccgccgctccgtgatcagggaggggtggttggggcttctggacgccgatcaaggcacacgtacgtcccctggcatggcgcaccctccctcgagctcgccggcacgggtgaagctagacgtcattgagcgctgaatcgcagttagcgaccgggccaaggttctcgccggcgtgatgagatggaacactttgactcccgcggccggatcggcctgtgttcgttcttgtttccgatctcccttctctttcccgttgcttcgatcccgtcaagtatggtagaccgtagcctattgttatgtttaaatgtcaattattatgtgtaattcctccaagcgccgatatccaataaggacgaaccggattttcgttagctcgacctcgaatgagaattttgtatacaatgcatcctcgttagctatgttcatctgttcgaatgcttgtgccctcatgttttcattccgttcgtcctctacacgaatggtgatcactatgtattgtgaacgagctcagtcatgtaggagctgccagattg

SEQ ID NO: 75 - DNA, 8124 bp, Zea mays
  source 1..8124: mol_type = unassigned DNA
tacgaatctagagattcgtctacaatgatttgaggacaggtgtgagttttcaaaagaaaatgctttcaaaaaagtatgatgaagggttttcacccttatcacctttgagtagggatgatcagggactccctggtttaggggagggcctaaggtgatggatcagctggtttaggtgtgagcagaaggattgtcccctcacataaggaccggtttgtcatccttcactacctgtactcatgataagtacaaccactcgagactatgtgggcagtcactcaatctgaactcgtacggtccaaccctagggttatgaaggttggggaacaccgggaggataaggagggggaatgttttgtccggtttggacatggtggtggcctgactccttccggtataaccgttaaggttaggatgtgcgaggaaagaaagagattcggcattcgggtctcacgacggtgagatcgcagaaatcggactagtggataaagtgtacatctctgcgcagagtttgaaaatctattcgaatagtccgtgtccacaggaatggtcgagtctggtatggtatggcaattaatgttttgttttcaaaaaagggtgcatttgagaaaaatgtttttaaaaagtccgacggttgagccgtgagctatggtggacgggaagtccagtagctgtttttgaaaatgaaaaccagtgggaaactgctgagatacctggatggtttagtccaggggattttgttctatattgaaaaacttcctgctcctttgggagaggatgcgctttgcaaaatacaaaatgttttacaaaacaaccccgcataaaatattgttgtttctgcaaaatatcctgagctccagatattccatgcattatatctgatttccccattccgcgggtgaaggtgggctgctgagtacgtttgtactcacccttgcttatttgttgtttttcagaaaaaggagatcgggtaagagttacgactgttcccaaccttgcctgtggttgttggaccgctgaattgcttcgatgcgtatatcgggctgcttcagccccactttgatgatatgtccctagttgtggaccaactcttaaagttgttcgccacctttataggtttgtatcgtttaagcagatctgtaatcatctgatgtataaatgtgtttactagcctcctgggactagtaattgtatcacatttgagtcccagaggattggggcgcttcattgtacatggcttgtggctcctctccttggttgaggatgaattgaccgagctccccctcgatcgtctcccgcttggtgatcttggtcacctcatccccttcatgtgcgatctttagaacatcccaaatctctttggcacttttcatcccttgcaccttattatactcctctcgacatagagaggcgaggagtatagtggtggcttgggagtcaaaatgccagatttgggcgacctcgtccgagtcatagccttcatcccccacagatggtacctgcgctccaaactcaacaatgtcccaaatgctagagtggagtgaggttagatgatgcctcattttatcactccacatacaataatcttcaccgtaaaaaaccggtggtttgcctaatgggacagaaagtaaaggagtgcgtttagaaatgcggggatagcgtaggggaatcttactaaacttcttgcgctcatggcgcttagaagtgacggacgacacgttggagccgtaggtggatgacgacgaagagtcagtctcgtagtagactaccttcttcatcttcttctttttgtcgccactccgatgcgacttgacgcgagaaggtgattcctcccttcctttggcgccagactcctttgatggagccttcccgcggcttgtgtctgtttccatctccctcttagcggatcctcccgacaccactttgagtggttagtctctaatgaagtatcgggttctgataccaattgaaagtcgcctagagggggtaaataggcggaaactgaaatttacaaattttaggcacaactataagccgaggttagtgttagaaataaaaccaagtccgaaagagagagcgaaaacaaatcaaccaagaaataagcgagtgacacggtgatttgttttaccgaggttcggttcttgcaaacctactccccgttgaggtggtcacaaagaccgggtctctttcaaccctttccctctttcaaacggtcacctagaccgagtgagcttccttccttgatttcccgagtcacttagaccccgcaaggaccaccacacaattggtgtctcttgcttcgcttacaaggctttgagagtaagaatgagagaaagaagaaagccaaccaagcaacaagagcaacaaaagaacacaagtcgatcttctcacaagtcctaaaaactaagttgaattgtggactttgatttgatcggaggctttgatttgtgtcttggagtgttgtgtattgctcttgtattgaatgaggagtagtgaatgcttaaatcttgaatggtggtggttgggggtatttatagccccaaccaccaaaacagccgttggggagggttgctgtcgatgggcgcaccggacagtccggttcgccaccggacactgtccggtgcgccaaccacgtcacccaaccgttagggttctgacggtttcgaccattggagctctgacatcttggtgcaccggacagtctggtgccgcaccgtacaggcattgtacactgtccggtgcgcctctggcgcctgctctgacttctgccgcgactgtagctttgttagggcactgtgcagtcgatcgttgcgctgatagccgttgctccgcttggtgcaccggacagtccggtggcacaccggacagtcaagtgaattacagcggagtgcgcctggagaaacccgaaggtgaagagtttgaggtcgatccaccctggtgcaccggacactgtccggtgcgccagaccagggttctcttcggtttcttttgctcctttcttttgaaccctaactttaatctttttattggtttgtgttgaacctttagcacctgtagaatatataatctagagcaaactagttagtccaattatttgtgttgggcatttcaaccaccaaaatcatttaggaaaaggtttgaccctatttccctttcacgtatgtaatcatttcaatatgaaataatgaataacaaaatgggccaatacaatttcattttgccaagtttttattacctttttacaccttgtcccattcatgaatcccctcttggaagattaataaatgggtgaaagggaatattttatttgtgttgccttgtttttatattttttaggaataaaaaacaagtgaccaatattggcatccacctacggtaaattcacaaccacaacgaagagtgtggcaagctgtgtagcttcatcatcgggctcaaagaagcttgtcttccccatcaccgggcgatcgatcatacttcgaactaaacaacaaaagatagaagaaagaagtccaaagaaaggtacaacttgttggtgttcaagggccctacattagatctaagaggtctcatattccaatcacattatctcgagaagaccttcagatcaaatattacccacacaaagatgctatggtaatcttgtatgtcataaaagacttcgtagtccgcaatgtcttggtcgacacaggcagtgcagtcgatatcatctttacgaaagcgttcaaactgtagaaaacagtgatgcctaagagagggatgaattaggacatctaaaaactatgtctaaacaaggccacaattaaatctctagagcaaagcctatgcaaaaaaaacaatctagaatgtgcaaactaggttttgtctaagtgtcgctatctctattgcaaagtctaagtttcaatcataaataatctaactagaaaggcgaggttgaaacttacatacttaatataaatacggaaggtaaagagtaaggtagatatgcaaactctcgtggatgacgccagtatttttattgaggtatctgaaaccacgcaaaggttccgactaatcctcgttggtgcccctacgcaaatggtagcccacacgaggtccaagcacctcggtcaagtaactccgtagagagccacatgccttctccacgcgcaagtggtgctctgctttcggctcctctcggacgctccccgacgtctccactatcgagcttccagctgaaatatcgtgggcctcgttccctccggtacactatggcgaccgtgacacaaactcggttgtcacgatcttgcaagactatcgccccacttgatacaattacaacgactcgcacaagagctgaggggttgtgtgatttttctaaacccacccaactaactaggattcaccaagagcaagcgcataagtggtctaactaacttaagcacttcgcgaagaacctacgctaatcactgagtgattctattaagcaatagggtgtttgagcacttggattgtctacaatatgccttgatatgttgcttaggctcccacaccttcaaatggccggttttgggggtatttataggcttccccctcaattatagccatcagacagaaagctgttatttctgtcgacgggcgcaacggacatgcattgttcactgtccagtgccatagccacgtcagtcgaccgttagggtctgtagcagtcgaccgttggatccgaccattaactagactgtttggtgcacaccggatagtctcgtgctacaaccaagagcgcttggttgtgggcctcacagcgcatactgcccggtgtcccaccggacaggtactgcttgtctggtccgccaccagtgcgctggctgactacccactttatggatttctttgttgtttccttgggcttcttttgttcttgagtcttggacttctacgtttcttttatgccttcttttgaggtgttgcatccgtagtgccttagtccaatcctcttcgcatcctgtggactataaatacaaacactagaacataagttgctttcgatatcattgatatggagttcccatacaacgcaatcattggaagaggagcacttaatcctttcaaagtagtcctagatttagcttatatttgtatgaagatatcaagcaagtaggacattatatcagcatataaaagccaagaagccacgagaagggtcgaaggaacctggcaagaatacaaggccatccataatactgattaaaccgaagatcaagcacaagacaaacaagttaaacaaaaatagttttggcagattagccgaagtaaattctcctctgtgaagatgtggccgaccaaagggttttgttcggttcacaatcaacctcagaacaagagacatgccttaaaagattcatgtttaacaacaatcatgtcttcgcttggtgagccaacaatctatgtgggtttgataggagcatcatagagcatgcactcaatgttgatccaagtattaaaccaagaaagcaaaagcttcgaaagatgtccaatgataagaccgacgttaaaaggctccttggtgctagagtaataagagaagttgcctacctagaatggcttgctaacacatgcatggttaagaaaccaaatggaaaatggaggatgtgcattgatttcacagatcttaacaaaacttgcccgaatgataaattccccttcccagggattaactcccttgtatatgtagcatacacttcggagctcatgagctttctagattgttattcaaggtatcatcaaatttggatgagaaaagaagacgaaccaaagaccaacttcataacccctagtagaacttattattacctttggatgcctaaagagctgaagaatgctggtggaacgttcaacagaatgacagacaaagtcctcaacacacaaattgggagaaatgtactaacatatgtggatgatatcattgttagaagcacaagacaagaagaccaaaattcatatttacaagaaacctttgtcaacttctgaaaagctggtatgaagtttaatcccgagaaatgtgttttcgtggtgaaggggaaattctttggctgcctcatgtcgaccaaaggaattgaagcaaacccccacaacatagaagctatcctatgaatggagctaccgaagtcaagaaaaggggctcagctgttggtaggcagacatgcttccttgaatagattcatctcaatatctgcataatgaagcttgctattctttgaagtgctaaagtcagctgaagtatttcaatggggaccactcaacatcaagccttcaaagggtttaagcagcatctaattcacttgacaactctcgtgctttctcatgcgtaaaatataaaggagaggttcttgggatcggttacgcttattgctttctcatgctccagcctccgtctttcctttccctaggagcttttctgccgtcgcgcctccttgctcgtgtgtccgtcctgcttgctcctcgcgcccatggcctccttcccatcctgctcaattcgtagagctaccttcttggtctagagaagatggggttcatccccttaacgaaggtctcggggtggagattggaaaatgagggtgaggtgttgtatccaagggacgacgaggtgtcgtgcttgcgtcctgctacaagcatgggtttggcctgccccttcgtcccttcgtatgggggatactccactactattagctggagatccagaatctccaccccaacaccgtcctccatatagcgtgttttattacgctatatgaggcttttatgggcatcgatccctattggaagttgtggcagtatctctttagcgcgtgggtgactttgggtcgtggcggtcaatcgtttggtggctcagcctccatccaactcctctctagctggaaggcagagtatttcaagatcttgcttccctccatcattcggtacgagggtgagtggttctacgccaagaatctgcccggcagtgctgtaccgtacatcggatgggagccgctttcgacgaataagtggcaccatggcacggatgcacgttccaagagctaagtggagcagcttctaaaggcgatcaccacgttgaagcagcacggtcttaccagcgtgtggcttatgcgtgttttaatgcaacgccgggttaagcctcagatggcctgctaaaacccgttgtacaagtactctagcgtcgacaaccccggccgccattcctccaagcctcttgcgctgaccgagatcgagactgcgggtctaggctatcaccgttctattgttgtgggcctttatggacgagaatttgcctcacccactttccaaagttgttctgatatgctttgtggtaggtatatctatttatcctcttttcatgttatcaaattacatatcaaatctcatcctccacaggctcaccagtcaccaacatcaatcgttctttgtgaaaccctgtgctggaatacacacgctcaatacattagttggtacatgagtctaatgaagtcagggaaagagtactaattttcgcgagtaaagggaaggctggatcctttttagttgcattgatcttggcttggtagtcctccccactcgcgacatctctctagccgcacgctggactacagagttagttccgcaagctataaaattcggctcgccattggcttgcaagattgagtagtgagccgtggacgcagctccccacttctcatcgccccctttttttaatttgtggccatctttggggtggtgggcggaggatttctaactggatggtgaagtttgtctggcgaaaaggacggctgcgacgaacccgtccatcgatccaacgctgtgcgcgcgttgggggagggacctgccaggccccacctgcagcgacagactattgatagatgccttcctctctgatcacctgatggctgatgccttcgcggccgtcttcgcctgccgctgctactactagttgccttcctcgcttccccgtctcgccccagccgcttcccccctcccctaccctttccttccccactcgcacttcccaaccctggatccaaatcccaagctatcccagaaccgaaaccgaggcgcgcaagccattattagctggctagctaggcctgtagctccgaaatc

SEQ ID NO: 76 - protein, 630 aa, Zea mays
MKREYQDAGGSGGDMGSSKDKMMAAAAGAGEQEEEDVDELLAALGYKVRSSDMADVAQKLEQLEMAMGMGGVGGAGATADDGFVSHLATDTVHYNPSDLSSWVESMLSELNAPPAPLPPATPAPRLASTSSTVTSGAAAGAGYFDLPPAVDSSSSTYALKPIPSPVAAPSADPSTDSAREPKRMRTGGGSTSSSSSSSSSMDGGRTRSSVVEAAPPATQASAAANGPAVPVVVVDTQEAGIRLVHALLACAEAVQQENFSAAEALVKQIPMLASSQGGAMRKVAAYFGEALARRVYRFRPPPDSSLLDAAFADLLHAHFYESCPYLKFAHFTANQAILEAFAGCRRVHVVDFGIKQGMQWPALLQALALRPGGPPSFRLTGVGPPQPDETDALQQVGWKLAQFAHTIRVDFQYRGLVAATLADLEPFMLQPEGDDTDDEPEVIAVNSVFELHRLLAQPGALEKVLGTVRAVRPRIVTVVEQEANHNSGTFLDRFTESLHYYSTMFDSLEGAGAGSGQSTDASPAAAGGTDQVMSEVYLGRQICNVVACEGAERTERHETLGQWRSRLGGSGFAPVHLGSNAYKQASTLLALFAGGDGYRVEEKDGCLTLGWHTRPLIATSAWRVAAAAAP

SEQ ID NO: 77 - DNA, 18 bp, Zea mays
  source 1..18: mol_type = unassigned DNA
cgcatatgggtgtcggcg

SEQ ID NO: 78 - DNA, 22 bp, synthetic construct
  misc_feature 1..22: note = gRNA
  source 1..22: mol_type = other DNA
gccttacaccggtcctcagcga

SEQ ID NO: 79 - DNA, 20 bp, synthetic construct
  misc_feature 1..20: note = gRNA
  source 1..20: mol_type = other DNA
gaagacacacgaggctgcct

SEQ ID NO: 80 - DNA, 21 bp, synthetic construct
  misc_feature 1..21: note = gRNA
  source 1..21: mol_type = other DNA
gctatatatggtgtatataag

SEQ ID NO: 81 - DNA, 20 bp, synthetic construct
  misc_feature 1..20: note = gRNA
  source 1..20: mol_type = other DNA
gttacggtgtgggcaatgtg

SEQ ID NO: 82 - DNA, 21 bp, synthetic construct
  misc_feature 1..21: note = gRNA
  source 1..21: mol_type = other DNA
gttctcagggtgaactaaaca

SEQ ID NO: 83 - DNA, 21 bp, synthetic construct
  misc_feature 1..21: note = gRNA
  source 1..21: mol_type = other DNA
gaatacatctctcacatatta

SEQ ID NO: 84 - DNA, 21 bp, synthetic construct
  misc_feature 1..21: note = gRNA
  source 1..21: mol_type = other DNA
gatacaacacacgttgttgcg

SEQ ID NO: 85 - DNA, 18 bp, synthetic construct
  misc_feature 1..18: note = gRNA
  source 1..18: mol_type = other DNA
gaaacacgaggtcttgag

SEQ ID NO: 86 - DNA, 5840 bp, Sorghum bicolor
  source 1..5840: mol_type = unassigned DNA
atgtccaaggcgcgggctaatgttgctggggttgggcgtgtggacgaagccgtgcgtgcgtgcgtgcgtgcgtgcgtgcgtgcgcggctggcccgggggcagagcggcagcggcacagtgcgcgcgggcctgccccccgtgcagttgaaaagaaaggtgcctgtagtggctgctgtccgtgcatgcatgttcatgtcctgccatcagggctcgcccgcgatcgatccatcatcaggcaatgcaatgcaagcggccggcctgtttattgttgggactcgtacctgctgcctgctccccagctgcatgcacagctaggcctagttccgaatgaaaatttttttttggtagtgtagcactttggtttgtttgtgaaaaatattatctaatcatagactaactaggattaaaaaaaattatctcgtgatttaaagctaaattgtgtaattagtttttattttcgtttatatttaatgtttcatgcatgtgccacaagattcgatgtgatgtggaatcttgaaaactttttaattttcggggtgaactaaacaagaccctagctatatatatactacacattactactcctacttttgtttgttttactcctacaaaagaaaaatgagggaggaacctgaaactcgcatgcaccctgcaggggcctctattttaatttgcacttgtgtgctagctgatctcgttcatagtacgagtacttgcgttgcgtcgtcctgctcctttgaaacgagattcctgctggaaccaagattgcaatctctgccttgctttaaaaaaaaggcctcagttgagagtactttggccaagtacgaaaatattaattcccggtcaagagaaagccagaaaggtggcagcacaagtgtgtgtttattaacaaggcccatcctcttctgaactctgagctgaggcgtgttacttcttcgtcgttttatatccaccagtcaagcgtaggagtagactctgaatgttttactctgaataatgaacagcgactctgctaatactttctgatccacttctgacggcacagggtctactgtacttgccaggcatgtttgagatttcagtcaagcaaagggcacggtttctgttaccggtgatgattggattattggatgccgtaacacccagcattaattaatactagtagagatgaagcccattcccgtcgagatttccatcgcgaggatatagaatgcgtacgtccgtgtacacgactgttgggcgcatgtacacgtacgttcggacgtactgatccactgtcgagagtagggaacgatcgacgatcaggcaggcaggcaggtgctacagggaggttaaccttcgttcttcgttctctctctctcgcgctcgctcgtcgtccttgcaatgtggtgagatgacatctacacgtgcggtggggacgcccgatgctttgttaaactcggtgaaagccggccatgagagatggcagcagtggtagacagctgggacagtgacaagccttgcgtgcagtaagatcttctgcaccttgctacctcacccttccttccttccttccttcaagttttatttaccgcgcactcccactccacacacgcaccaccctggtatgatacacgaactgtagtagtaggagtattttttccagttgcgtgctcggtcttttccaaaaaagcctttgccatatacagtacgtgtgtgttgatagagtagcagtaactttcttttcttctgcaaactgattgatgatccaatccaaagcaagcaagcaagcaagcaacctgttagctgctgtatgtatgtacttgcaaacaacacaaagatgagtgttgagtaacataagctggtggctggccgccccacgcacaccccccaccccggcaccccgtagctatcggcctcggctaccatcccccgctcccgtcccgggcggcctcccgtgccctgcaactgcaatccccgaccccgacccggagcagccaccaccaccggcgcgcataaatgggtgtcggcgacccgcgagccagccccgcccaccaccacagacgatcccctcccccttccccccgccccagcagcaggagcagcagctagcctatccaacagtgaaaagcacgcgcgctccgcccggccctccctcctctcctgcggtgcgtgcgtgcggtgccgccgagcgatccatccatctgacgggagggtgcgtgagatgcctgcctgcctgcctgcctccctccctccctgtagctctggcctcttcctctagctccagcctccagccagccgtgcgggtttctgctgcagcggaagaagagcgagcagtgcgtgcgtgtcctcctgctcccacacgaactccatcgtcctcacctcagctcatctcccgcgcgccgcggccacccacacgcacgggcacgagcacgatgccgtgctcgtcggcggccccgacgtggctgctgcgggtggcgtcggcggccgaccaggcctcgtcctcgtcctcgtccaagggcggcggccgcgtgctcaccgccggcaccactggcaccaccatggacacggccgccaccgctgccgccgccggctgtggcggcaacggcggcgggggaggcggcagtaatgccgccgacctccaggagagcagcagcagcgggcagtcccggctcgcggcgcgcggccactggcgccccgccgaggacgccaagctccgcgagctcgtcgcgctctacggtccccagaactggaacctcatcgccgagaagctcgacggcagatccggtacgtacgcacgagtcatcatcatcttgtactgctctcactgctgacctcattcactcttgccatcgacggcgctccaccgatcgagatatatatatatatatatacaaatatacagaatacattggtcacctatatattggaaggagtatatacttgctgtttgacacatgtacatggtgtaatgcttggtgtggctgcgcgcatgcagggaagagctgccgcctccggtggttcaaccagctggacccgcggatcagcaagcggcccttcagcgacgaggaagaagagcggctgatggcggcgcaccgcttctacggcaacaagtgggcgatgatcgcgcgcctgttcccggggcgcacggacaacgccgtcaagaaccactggcacgtcatcatggcgcgcaagtaccgcgagcagtccacggcgtaccgccgccgcaagctcaaccaggcagtccagcggaagctcgaggcctccgccgcggcggtcgcaacaatgccgccggccgcgggcagcacgggagacgtcgtcggcgccgccctcggccaccaccaccaccaactcctggcggccgccgccgccgccgcccacgacgcggcctacggcttcgccgcggcggacccctacggcgccttcggcttccgccaatactacccgttcccgccagcttcggccgaggacacgccgccgccgccgccgcctcccttctgcttgttccctggtgagcactccggcaaatcatccctcccttcccgaccgccattacctagcacgacacgacaccgcattcgctcgcgctcgtacaccacggacggagacacggcggagtgtatgcgtatgcatgacagccggggcccgtcccctggcacagtaacttccctcccggctgattgttccctcccccccccccccccccccccccccaccgtgtccggccgccggcaacggcaacactagtggctgacagatgagcccgagaaggtcccggagcccatctatcagcgagtgtcacgggactgctctccccgtcgtgatggataccgatggattcctggccgccatggccggattcattttttactgtatgtatatgacgaccctagccggcctttgttatgtctgtccggccgtgtgacgatgacgagacctatctctatctacctgggcgcgctagctgtacctgtacctgtacggtttcagctgcagcgtttctactgtagcataggcctagagggaggaggaggaggagtctggccgggcacacgcagtacattcaggcacagcatcgatgaaccatgattccatgataggcagacacacacgggccgatcgaatcaatggaggaccgccgatcggggcggtgcactggtcgctgcacggcctccgatccgtccgatcgccaccgccggcccccctggcgggcgtaaatgcctttgcttgaaataggaagcgctgcgggcgggcgagatcgatctcgtcggcatgcatcggagacggagacggagaccaggcaggcatgcactgcacaggccggccgcgaagacttggacttgcagcacggcgccacagtgccagcagatgagagaggggtcatcagggagtcgtcgcgcggcgcagcgagcagtgaggcttttttagatgacgtgccaaggcgatgccttgctgccatcaatgcgggcgctagactagagctgcccccttgttggctgttgctctcacagtcacacactcatcactctatggtcacctgctgctgctgtgccgttggcttcgtctgttggccgtggctggccacacaactcatcacatacaagatctcctgtgtgcagtgcgtgcgtgttcttttctttgggagggcttttgtttgcttgcttcttgattaatttcccgtcctgattattattcgttattggtttcttgattgattgtgccattgctgcggcgtcgctttctggtggtctctaacttccaagaatgcgccacatacagccactagctacttcaatttgtcatcagtcgctataacaccactgtgtcacgccacgcgatcgaggtgtgcaaaaatctgcttgtcttttgatccatggcatatatatatacagggcccagcagcgcggcggcgcttcacgccgacagcaggcgccttccctggccgtcgtcgtcgtcgtcggatgctgccgctgccgccgccggtggcggcaggtacggggagccgcagcagcagctcctgctgcccgttgttcacggtggcagctggatcgacggcgtcggcgtggccgtggccggcggtcaccacgaggcgcagttcgtcttgggcaacaacgggggagcctttgaagggaccacaagacagcagggcgccgccgccggcgctcactttgaagctgccgcggcggcgccgccgccagcgttcatagatttcctcggtgtcggagccacatgaatgtgcatgcatgctccacttcaacgacacatgcgccttgcagttgcaggtggatctaagctaggacaagtagctcgggctcgatctagcagctagcgcaccactactagctagctacctttgccatatgagtacatccaaggcttagcttatgtagtagtagcacgcctctatctctatgtatgtatgtatgtatgtatatctacctaggagctggtgacggagattgagcttatttctatcggggctttgtagatgagggactatagggagggatgcttggactttgatcatttgggatcggatttagttacttgatgattagcccccagccatatatgtatgtatgtatgtatgtatgcaggcaagaaagatcgatgatggtgtgaattggagagctaaatttgcagtggtggattcttttagtactagccaaaagtgtagaagcatgaggtttgagtaaattcaaaatttgcatcactacaatttaaaattgcaacgtttcattagtgttgttgcataaaaaaaatcctacgaaaacatatggctatcaaatcagtacaagtcgaccatcattcccaataacagtggcatccatggatcttagtaggttttcttagaaatcaac

SEQ ID NO: 87 - DNA, 2052 bp, Sorghum bicolor
  source 1..2052: mol_type = unassigned DNA
atgtccaaggcgcgggctaatgttgctggggttgggcgtgtggacgaagccgtgcgtgcgtgcgtgcgtgcgtgcgtgcgtgcgcggctggcccgggggcagagcggcagcggcacagtgcgcgcgggcctgccccccgtgcagttgaaaagaaaggtgcctgtagtggctgctgtccgtgcatgcatgttcatgtcctgccatcagggctcgcccgcgatcgatccatcatcaggcaatgcaatgcaagcggccggcctgtttattgttgggactcttttatttaccgcgcactcccactccacacacgcaccaccctgctatcggcctcggctaccatcccccgctcccgtcccgggcggcctcccgtgccctgcaactgcaatccccgaccccgacccggagcagccaccaccaccggcgcgcataaatgggtgtcggcgacccgcgagccagccccgcccaccaccacagacgatcccctcccccttccccccgccccagcagcaggagcagcagctagcctatccaacagtgaaaagcacgcgcgctccgcccggccctccctcctctcctgcggtgcgtgcgtgcggtgccgccgagcgatccatccatctgacgggagggtgcgtgagatgcctgcctgcctgcctgcctccctccctccctgtagctctggcctcttcctctagctccagcctccagccagccgtgcgggtttctgctgcagcggaagaagagcgagcagtgcgtgcgtgtcctcctgctcccacacgaactccatcgtcctcacctcagctcatctcccgcgcgccgcggccacccacacgcacgggcacgagcacgatgccgtgctcgtcggcggccccgacgtggctgctgcgggtggcgtcggcggccgaccaggcctcgtcctcgtcctcgtccaagggcggcggccgcgtgctcaccgccggcaccactggcaccaccatggacacggccgccaccgctgccgccgccggctgtggcggcaacggcggcgggggaggcggcagtaatgccgccgacctccaggagagcagcagcagcgggcagtcccggctcgcggcgcgcggccactggcgccccgccgaggacgccaagctccgcgagctcgtcgcgctctacggtccccagaactggaacctcatcgccgagaagctcgacggcagatccgggaagagctgccgcctccggtggttcaaccagctggacccgcggatcagcaagcggcccttcagcgacgaggaagaagagcggctgatggcggcgcaccgcttctacggcaacaagtgggcgatgatcgcgcgcctgttcccggggcgcacggacaacgccgtcaagaaccactggcacgtcatcatggcgcgcaagtaccgcgagcagtccacggcgtaccgccgccgcaagctcaaccaggcagtccagcggaagctcgaggcctccgccgcggcggtcgcaacaatgccgccggccgcgggcagcacgggagacgtcgtcggcgccgccctcggccaccaccaccaccaactcctggcggccgccgccgccgccgcccacgacgcggcctacggcttcgccgcggcggacccctacggcgccttcggcttccgccaatactacccgttcccgccagcttcggccgaggacacgccgccgccgccgccgcctcccttctgcttgttccctgggcccagcagcgcggcggcgcttcacgccgacagcaggcgccttccctggccgtcgtcgtcgtcgtcggatgctgccgctgccgccgccggtggcggcaggtacggggagccgcagcagcagctcctgctgcccgttgttcacggtggcagctggatcgacggcgtcggcgtggccgtggccggcggtcaccacgaggcgcagttcgtcttgggcaacaacgggggagcctttgaagggaccacaagacagcagggcgccgccgccggcgctcactttgaagctgccgcggcggcgccgccgccagcgttcatagatttcctcggtgtcggagccacatga

SEQ ID NO: 88 - protein, 683 aa, Sorghum bicolor
MSKARANVAGVGRVDEAVRACVRACVRARLARGQSGSGTVRAGLPPVQLKRKVPVVAAVRACMFMSCHQGSPAIDPSSGNAMQAAGLFIVGTLLFTAHSHSTHAPPCYRPRLPSPAPVPGGLPCPATAIPDPDPEQPPPPARINGCRRPASQPRPPPQTIPSPFPPPQQQEQQLAYPTVKSTRAPPGPPSSPAVRACGAAERSIHLTGGCVRCLPACLPPSLPVALASSSSSSLQPAVRVSAAAEEERAVRACPPAPTRTPSSSPQLISRAPRPPTRTGTSTMPCSSAAPTWLLRVASAADQASSSSSSKGGGRVLTAGTTGTTMDTAATAAAAGCGGNGGGGGGSNAADLQESSSSGQSRLAARGHWRPAEDAKLRELVALYGPQNWNLIAEKLDGRSGKSCRLRWFNQLDPRISKRPFSDEEEERLMAAHRFYGNKWAMIARLFPGRTDNAVKNHWHVIMARKYREQSTAYRRRKLNQAVQRKLEASAAAVATMPPAAGSTGDVVGAALGHHHHQLLAAAAAAAHDAAYGFAAADPYGAFGFRQYYPFPPASAEDTPPPPPPPFCLFPGPSSAAALHADSRRLPWPSSSSSDAAAAAAGGGRYGEPQQQLLLPVVHGGSWIDGVGVAVAGGHHEAQFVLGNNGGAFEGTTRQQGAAAGAHFEAAAAAPPPAFIDFLGVGAT

SEQ ID NO: 89 - DNA, 1418 bp, Sorghum bicolor
  misc_feature 1042: note = n is a, c, g, or t
  misc_feature 1053: note = n is a, c, g, or t
  source 1..1418: mol_type = unassigned DNA
atgccgtgctcgtcggcggccccgacgtggctgctgcgggtggcgtcggcggccgaccaggcctcgtcctcgtcctcgtccaagggcggcggccgcgtgctcaccgccggcaccactggcaccaccatggacacggccgccaccgctgccgccgccggctgtggcggtggcgcctgcggcgggggaggcggcagtaatgccgccgacctccaggagagcagcagcagcgggcagtcccggctcgcggcgcgcggccactggcgccccgccgaggacgccaagctccgcgagctcgtcgcgctctacggtccccagaactggaacctcatcgccgagaagctcgacggcagatccgggaagagctgccgcctccggtggttcaaccagctggacccgcggatcagcaagcggcccttcagcgacgaggaagaagagcggctgatggcggcgcaccgcttctacggcaacaagtgggcgatgatcgcgcgcctgttcccggggcgcacggacaacgccgtcaagaaccactggcacgtcatcatggcgcgcaagtaccgcgagcagtccacggcgtaccgccgccgcaagctcaaccaggcagtccagcggaagctcgaggcctccgccgcggcggtcgcaacaatgccgccggccgcgggcagcacgggagacgtcgtcggcgccgccctcggccaccaccaccaccaactcctggcggccgccgccgccgccgcccacgacgcggcctacggcttcgccgcggcggacccctacggcgccttcggcttccgccaatactacccgttcccgccagcttcggccgaggacacgccgccgccgccgccgcctcccttctgcttgttccctgaaccatatgttgaggctccatttgctcccgttcaacctcaccatgcgcgcgtggggaagatgagccagcgccaacggacgggatgtgaatcgaggattgcgttaactgcaggctgcgcgtggggaagatgagccagcgttaattgtaggctgagcatgggttgcgttaactgcnggccggggggngcttgcatgcagccatgcagagggggcccagcagcgcggcggcgcttcacgccgacagcaggcgccttccctggccgtcgtcgtcgtcgtcggatgctgccgctgccgccgccggtggcggcaggtacggggagccgcagcagcagctcctgctgcccgttgttcacggtggcagctggatcgacggcgtcggcgtggccgtggccggcggtcaccacgaggcgcagttcgtcttgggcaacaacgggggagcctttgaagggaccacaagacagcagggcgccgccgccggcgctcactttgaagctgccgcggcggcgccgccgccagcgttcatagatttcctcggtgtcggagccacatga

SEQ ID NO: 90 - protein, 332 aa, Sorghum bicolor
MPCSSAAPTWLLRVASAADQASSSSSSKGGGRVLTAGTTGTTMDTAATAAAAGCGGGACGGGGGSNAADLQESSSSGQSRLAARGHWRPAEDAKLRELVALYGPQNWNLIAEKLDGRSGKSCRLRWFNQLDPRISKRPFSDEEEERLMAAHRFYGNKWAMIARLFPGRTDNAVKNHWHVIMARKYREQSTAYRRRKLNQAVQRKLEASAAAVATMPPAAGSTGDVVGAALGHHHHQLLAAAAAAAHDAAYGFAAADPYGAFGFRQYYPFPPASAEDTPPPPPPPFCLFPEPYVEAPFAPVQPHHARVGKMSQRQRTGCESRIALTAGCAWGR

SEQ ID NO: 91 - DNA, 1205 bp, Sorghum bicolor
  source 1..1205: mol_type = unassigned DNA
tgccgtgctcgtcggcggccccgacgtggctgctgcgggtggcgtcggcggccgaccaggcctcgtcctcgtcctcgtccaagggcggcggccgcgtgctcaccgccggcaccactggcaccaccatggacacggccgccaccgctgccgccgccggctgtggcggcaacggcggcgggggaggcggcagtaatgccgccgacctccaggagagcagcagcagcgggcagtcccggctcgcggcgcgcggccactggcgccccgccgaggacgccaagctccgcgagctcgtcgcgctctacggtccccagaactggaacctcatcgccgagaagctcgacggcagatccgggaagagctgccgcctccggtggttcaaccagctggacccgcggatcagcaagcggcccttcagcgacgaggaagaagagcggctgatggcggcgcaccgcttctacggcaacaagtgggcgatgatcgcgcgcctgttcccggggcgcacggacaacgccgtcaagaaccactggcacgtcatcatggcgcgcaagtaccgcgagcagtccacggcgtaccgccgccgcaagctcaaccaggcagtccagcggaagctcgaggcctccgccgcggcggtcgcaacaatgccgccggccgcgggcagcacgggagacgtcgtcggcgccgccctcggccaccaccaccaccaactcctggcggccgccgccgccgccgcccacgacgcggcctacggcttcgccgcggcggacccctacggcgccttcggcttccgccaatactacccgttcccgccagcttcggccgaggacacgccgccgccgccgccgcctcccttctgcttgttccctgggcccagcagcgcggcggcgcttcacgccgacagcaggcgccttccctggccgtcgtcgtcgtcgtcggatgctgccgctgccgccgccggtggcggcaggtacggggagccgcagcagcagctcctgctgcccgttgttcacggtggcagctggatcgacggcgtcggcgtggccgtggccggcggtcaccacgaggcgcagttcgtcttgggcaacaacgggggagcctttgaagggaccacaagacagcagggcgccgccgccggcgctcactttgaagctgccgcggcggcgccgccgccagcgttcatagatttcctcggtgtcggagccacatga

SEQ ID NO: 92 - protein, 401 aa, Sorghum bicolor
MPCSSAAPTWLLRVASAADQASSSSSSKGGGRVLTAGTTGTTMDTAATAAAAGCGGNGGGGGGSNAADLQESSSSGQSRLAARGHWRPAEDAKLRELVALYGPQNWNLIAEKLDGRSGKSCRLRWFNQLDPRISKRPFSDEEEERLMAAHRFYGNKWAMIARLFPGRTDNAVKNHWHVIMARKYREQSTAYRRRKLNQAVQRKLEASAAAVATMPPAAGSTGDVVGAALGHHHHQLLAAAAAAAHDAAYGFAAADPYGAFGFRQYYPFPPASAEDTPPPPPPPFCLFPGPSSAAALHADSRRLPWPSSSSSDAAAAAAGGGRYGEPQQQLLLPVVHGGSWIDGVGVAVAGGHHEAQFVLGNNGGAFEGTTRQQGAAAGAHFEAAAAAPPPAFIDFLGVGAT

SEQ ID NO: 93 - DNA, 11417 bp, Sorghum bicolor
  source 1..11417: mol_type = unassigned DNA
gttaaataataattgtcaaatacaaatgaaagtgttatagtattgaaatcataaaacttttttatatgtcttgtttagttcacccttagggcttgtttagttcccaaaaaaaattgcaaaattcttcaaattccccatcacatcgaatctttagacgtaagcatggagtattaaatatagacgaaataaaaaataattgtacagtttggtcagaattgacgagataaatcttttgagtctagttagtccatgattggataatatttgtcaaatacaaacgaaaaaactaaggccttgtttagatgcacctaaaaacccaaaactttacaagattccccatcacatcgaatcttgtggcacatgcatggaatattaaatatagataaaaaagataactaattatacagtttacctgtaaatcacgatacaaatctaaaaagacgaaaatgctacggtgtcaaaatctaaaaagtttttgcatctaaacaagggccttgtttagttcgcaaaatttttaagatttcccgtcacatcgaatctttggtcgtatgcatggagcattaaatatagataaaaataaaaactaattgcacagtttacctgtaatttgtgagatgaatcttttgagcctagttactccatgattggacaatgtttgtcaaataaaaacaaaagtgctacagtagtcaaaaaccaaagtttttgccaactaaacgaggcctaggcctaattgcacattttgactgtaaatcgcgagacgaatctttgaaaactttttatacgtcttgtttagttcacccttaaaaaccaaaaattttttcaagattctccgtcacatcgaatcttatggcacaaacatgtgcactaaaaatagatgaaaacacaaactaattgtacagtttgtctgtaaatcgcgagacgaatcttttaaatctagttactgtatgattgaataatgtttgttaaataaaaatgaaagtggccttacaccggtcctcagcgaaggaagaaacacgaaattcttgtagagcacaagcacaaaaggttttccatcggccatagattttcgtgacgacactgatagaacgcaacgcgtagaatacatccattctcgttcgttttccacagtatggggatgagtactgtactgtgcaaggagcccccccgtacattcacagtctctctcactcgttggactcttcctctactcctacaaagacacacgaggctgcctgggattggagccttgcactgggccgacgccgaccggacggccgagcgaacgagcctgaaccagagcccctctcatcagaggtctcaagccgcaagccaattataagaggcgagacaaagcaactcccaattcaatccaccccagggcctccctccctccctctcggcatcctctgctttgctctgcgccagccactctgccgaggtgggggcagaggagaggagagcttccccccctcccttccctcggtccctccccggcccccgatcgatgtctaccaacgacccggacgagatcagggcgcgcgtcgtcgtcctcggcgcccctcatgccgacgacgacgccggcgacgagtgggcccgccccgagctcgaggccttccacctcccctctcccgcccaccagcctcctggcttccacctagccgctgggcaccaaccggaagctgcagcagagcaacccaccacgctccctgctgcccgccgcaccagcgacacatccactgctgctggtgctgctcctccttctccttcgccgcctccgccgccggctcctttggagatggaccagccgcccaatgccaagccggcctcctcctccgccgccgccgccggcgccaatgacaacaagaagcccaccccgcccgccgcgctgcgcgacctcttccgcttcgccgacggcctcgactgcgcgctcatgctcgtcggcacgctcggcgcgctcgtccacggctgctcgctccccgtcttcctccgcttcttcgccgacctcgtcgactccttcggctcccacgccaacgacccggacaccatggtccgcctcgtcgtcaagtacgccttctacttcctcgtcgtcggagccgcaatctgggcgtcctcatgggcaggtaaccaacgttattcctcctcctcctcctcttcctcctcccggcactgctgctcgcgtcgcgaattgtctgtcgatttggattggatggcgaatcacatcagtcgctcaatcttcatggcccatggctagcaatgagatcgaccttcgaatccctcgcttgcagagatctcctgctggatgtggaccggcgagcggcagtcgacgcggatgcggatccggtacctggacgcggcgctgcggcaggacgtgtccttcttcgacaccgacgtgcgcgcctcggacgtcatctacgccatcaacgcggacgccgtggtggtgcaggacgccatcagcgagaagctgggcaacctcatccactacatggccaccttcgtggcgggcttcgtcgtgggcttcaccgccgcctggcagctggcgctcgtcacgctcgccgtcgtgccgctcatcgccgtcatcggggggctcagcgccgccgcgctcgccaagctctcctccaggagccaggacgcgctgtcgggcgccagcggcatcgcggagcaggcgctcgcgcagatacggatcgtgcaggccttcgtcggcgaggagcgcgaaatgcgggcgtactcggcggcgctggccgtcgcgcagaagatcggctaccgcagcggcttcgccaaggggctcggcctcggcggcacctacttcaccgtcttctgctgctacggcctcctgctctggtacggcggacacctcgtccgcggcaaccacaccaacggagggctcgccatcgccaccatgttctccgtcatgatcggcgggctgtaagatgatcagtttctcccgggctctcctgttcttccgtcatgacacagcatgtactacgtacgcttactggtctgtgtctgtgtgtgtgtggatcgcgtgcgtccagggccctcgggcagtcggcgccgagcatggccgcgttcgccaaggcgcgcgtggcggccgccaagatcttccgcatcatcgaccacaggccgggcatctcctcgcgggacggcgaggacggcggcggcgtggagctggagtcggtgacggggcgggtggagatgaggggcgtggacttcgcgtacccgtcgcggccggacgtccccatcctgcgcggcttctcgctcagcgtgcccgccggcaagaccatcgcgctggtgggcagctccggctccgggaagagcacggtggtgtcgctcctcgagaggttctacgaccccagcgcaggtatacatagtacgctaccaattctagctttagcgcattgattaattagtgttggagttcacttgcttgccaattgccattgccatcacacatcagcagctaccatacattgccaactgccattgctgctgccttgctgggtggttagtaggggaagaagcttccactgtagcaggagtacattgcaaacaggaagtgaattttgcagtgggaaatgaagaagtgaatgcttggagcagagctggccggcctcatgggctgcttacctactagctagtcaaccaagcatcctgtttcttccttgtttatggtcatgcattcacacagctagaactagaagagctagccttgtttagtttctagaaaattttgtaaaatttttaagttccctgtcacatcgaatctttagacgcatgcatggagtattaaatatagacgaaaataaaaactaattgcacagtttggtcgaaattgacgaaacgaatcttttgagcctaattagtccatgattggacaatatttgtcaaatacaaacgaaagtgctgccgtatcgattttgcaaagtttttcggaactaaacaaagcctggtgcctgcaacgcgagacaaagaaaactatttgcctggcaagatgccactattgcacatgcatgccactctttgagccttgaccgactgactgactactcagagtaggagtggttcaattgtattgacatgtagtaggagtactcgtatgctatagtagtcctgtagttttttcaaacaaaaaaaaaagagaaagaaagaaatgaagtctgaaatttgttggttttggcagggcaaatcttgctggacgggcatgatctcaagtcgctgaagctccggtggctccggcagcagattggtctggtgagccaggagccgacgctgttcgcgacgagcatcaaggagaacctgctgctggggcgggacagtcagagtgcgacgcaggccgagatggaggaggccgccagggtggccaacgcgcactccttcatcgtcaagctccccgacggctacgacacgcaggtccgtatcgtatagctagctcctagctgcactgtgctgcccttttcttgctcgccaccgttgctgcctgacatgtttgtgcctgttgttctccaacccatttgtcagtgtctagaccaccatgcctgcctgcctagctactcgctgctccccgtctccttcctttcgctaccctgccttttttctcatctgcatgcagcctttttcttttctccatgtcatgagaggatgcagcatgttgcatcatgtcatccatctccattcatgcgtcgcgctcgtgcctgcccggggtagcctcagatcagatactgtttgtgtttgttaggccgcggcagcggcacatgagcagctcatgacaggcagcaccaccaacgccatgtccgccatggaggtgtcgggtccatgtccattccatcacagcacggcactactcatccatcacatagataccagtagacagacactagtactagtactagaactagggatgggagagcaattgcttgttaggagcctgttgctatcacgattgctgttgtgggttaccaacaagtaacatgccagggatgctttgctatcacacacaggacaggagaggtccctttctcaacacgaaactctactagcagtttagcacttgctgctgagtgctgagagcagagggcagagggagatgaatggatggtgagcagctagaggaagagccagaaaaggttaataacaatagtaaaaagattaagaatcaacctggggtacgtagaaagaggtagaattcctaagaataatataggagtgggagtggaacagaacaaattccaagctggtatttttgtcaggaatgtcaagttgatttgatcccagtgcaagcaagaattatcaatcacccatcctgtctgtacaatccagctcttgctactctacttagcactactgtgctactagtggtaggtatcttccacttctcttaatataatctgtcatgagagaaagagagtcagacaagcccatgctgctgcttattttaatcactgtcagtggcaggcagtctggtttgttaataacatctgggaagggtttaatcaaaccagatcaaatctaatgaaatctaagaggtcacatgggatatgggccacatagagcagggccttgtttagttcccaaaaaattttacaaaattttttagattctccatcacatcgaatctttagacacatgcatgaagtattaaatatagataaaaataaaaactaattgcacagtttggtcgaaattgacgagacgaatcttttgaacctagttagtctttgattggataatatttgtcaaatacaaacgaaaaagctacagtgtcgattttgcaaaatattttggaactaaacaaggcccagctcgccttcccacactcccatgactgattccttgctaagcaatgctcgcatgcccatgcatcatccctggtcaaacactctgcaccatcataagactagtattaacaaatgatttcatttttgttgttattaattatgctggaggagtggtactattttattatatttgatgaaaaacttggcagtcaaagtcaacccgtttgtttgacactgcgtgcatggccggtgcaggttggggagcgcggcctgcagctctccggcgggcagaagcagcgcatcgccatcgcccgcgccatgctcaagaaccctgccatcctgctgctggacgaggctaccagcgcgctcgactccgagtcggagaagctcgtgcaggaggcgctggaccgcttcatgatcgggcgcaccaccctggtgatcgcgcacaggctgtccaccatccgcaaggccgacgtcgtggccgtgctgcagggcggcgccgtctccgagatgggcacccacgacgagctcatggccaagggcgagaacggcacctacgccaagctgatccgcatgcaggagcaggcgcacgaggcggcgctcgtcaacgcccgccgcagcagcgccaggccctccagcgcccgcaactccgtcagctcgcccatcatgacgcgcaactcctcctacggccgctccccctactcccgccgcctctccgacttctccacctccgacttcaccctctccatccacgacccgcaccaccaccaccggacgatggccgacaagcagctcgcgttccgcgccggcgccagctccttcctccgcctcgccaggatgaactcgcccgagtgggcctacgcgctcgtcggctccctgggctccatggtctgcggctccttcagcgccatcttcgcctacatcctcagcgccgtgctcagcgtctactacgcgccggaccctcgctacatgaagcgcgagatcgccaagtactgctacctgctcatcggcatgtcctccgcggcgctgctgttcaacacggtgcagcacgtgttctgggacacggtcggcgagaacctcacgaagcgtgtgcgcgagaagatgttcgccgccgtgctccgcaacgagatcgcctggttcgacgccgacgagaacgccagcgcgcgcgtcgccgccaggctcgcgctcgacgcccagaacgtgcgctccgccatcggggaccgtatctccgtcatcgtccagaactcggcgctcatgctcgtcgcctgcaccgcgggcttcgtcctccagtggcgcctcgcgctcgtgctcctcgccgtcttcccgctcgtcgtcggcgccaccgtcctgcagaagatgttcatgaagggcttctcgggggacctggaggccgcgcacgccagggccacgcagatcgcgggcgaggccgtcgccaacctgcgcaccgtggcggcgttcaacgcggagcgcaagatcacggggctcttcgaggccaaccttcgcggcccgctccggcgctgcttctggaaggggcagatcgccgggagcggctacggcgtggcgcagttcctgctgtacgcgtcctacgcgctggggctctggtacgccgcgtggctagtgaagcacggcgtctccgacttctcgcgcaccatccgcgtgttcatggtgctcatggtgtccgccaacggcgccgccgagacgctgacgctggcgccggactttgtcaagggcgggcgcgcgatgcggtccgtgttcgagaccatcgaccggaaaacggaggtggagcccgacgacgtggacgcggcgccggtgccggagcggcccaagggcgaggtggagctgaagcacgtggacttctcgtacccgtcgcggccggacatccaggtgttccgcgacctgagcctccgggcgcgcgccgggaagacgctggcgctggtgggtccgagcgggtgcggcaagagctcggtgctggcgctggtgcagcggttctacgagcccacgtccgggcgcgtgctcctggacggcaaggacgtgcgcaagtacaacctgcgggcgctgcggcgcgtggtggcggtggtgccgcaggagccgttcctgttcgcggcgagcatccacgacaacatcgcgtacgggcgcgagggcgcgacggaggcggaggtggtggaggcggcgacgcaggcgaacgcgcaccggttcatctcggcgctgccggagggctacgggacgcaggtgggcgagcgcggggtgcagctgtcgggcgggcagcggcagcggatcgcgatcgcgcgcgcgctggtgaagcaggcggccatcatgctgctggacgaggcgaccagcgcgctggacgccgagtcggagcggtggctcttcgaggccaaccttcgcggcccgctccggcgctgcttctggaaggggcagatcgccgggagcggctacggcgtggcgcagttcctgctgtacgcgtcctacgcgctggggctctggtacgccgcgtggctagtgaagcacggcgtctccgacttctcgcgcaccatccgcgtgttcatggtgctcatggtgtccgccaacggcgccgccgagacgctgacgctggcgccggactttgtcaagggcgggcgcgcgatgcggtccgtgttcgagaccatcgaccggaaaacggaggtggagcccgacgacgtggacgcggcgccggtgccggagcggcccaagggcgaggtggagctgaagcacgtggacttctcgtacccgtcgcggccggacatccaggtgttccgcgacctgagcctccgggcgcgcgccgggaagacgctggcgctggtgggtccgagcgggtgcggcaagagctcggtgctggcgctggtgcagcggttctacgagcccacgtccgggcgcgtgctcctggacggcaaggacgtgcgcaagtacaacctgcgggcgctgcggcgcgtggtggcggtggtgccgcaggagccgttcctgttcgcggcgagcatccacgacaacatcgcgtacgggcgcgagggcgcgacggaggcggaggtggtggaggcggcgacgcaggcgaacgcgcaccggttcatctcggcgctgccggagggctacgggacgcaggtgggcgagcgcggggtgcagctgtcgggcgggcagcggcagcggatcgcgatcgcgcgcgcgctggtgaagcaggcggccatcatgctgctggacgaggcgaccagcgcgctggacgccgagtcggagcggtgcgtgcaggaggcgctggagcgcgccgggaacggccgcaccaccatcgtggtggcgcaccggctggccacggtgcggaacgcgcacaccatcgccgtgatcgacgacggcaaggtggtggagcaagggtcgcactcgcacctgctcaagcaccatcccgacgggtgctacgcgcggatgctgcagctgcagcggctgacaggcggtgccgcgcccgggccgccgccgtcgtcgtccaacggggccgccgcgtaggatggatggatggatcatggatgagtttggttccttgagagattgatggatgaggaagctgaagctccggagggaatgatggtactccatgatcgcaacaaggggaaaagaaaaaaagaagcagaaaacacggtggttcatatgattgtacaatttgatgatgatctctttgagttgaggttttaggatgatgtaaactttcacatctttttttttttgactctttttgcttctcattcttcttgtttctcatctgctagctgggactgagatgtggaacagatagaagcaacagcatctatctaaatacagtgccagagatgggaattgatccttccttgtaagtagacgatggctagctcccaacttcccaaggaaaaggaacccagatcatatgtactactgtacttatactgtattagttagcctcttatatacaccatatatagcagatttactgcttatttgcaatagcagcaacagatagggaccagaaaaatgaaggtagtgcatgaatgattgctactatcggtgagtactccctccgtatcaaattacaagtcattttaagaatcttggagagtcaaagatttttaagtttgaccaaatttatatagtaaaacaataatatttttggtatcaactaagtatcattagattctttgttagttatattttcatagtatgcctattttatgacataaatctttatatttttctatatatttttggtcaaacttgaaaatactttgactctccaagattcttgaatgacttataatttagaacggagggagtagcatgaaagaaatggcaagctccacttgtctctgaaacacaaaagcatctcgattttcagcggcagaggagctgaacagtatctatccttttccgtttctgtttcttctctctatttttgcgaggacactctccatccttttcaggcagacaggaagcggatctagtggtgagcagtgagcacgcctctcagaatttttttttatttttagcccatttttaaatcaaatttcaattttaggccgcttttgaaaaaaatttcaaaactaggccgtttggccccgccaccatgcatggcggggcaaaaccactacaccccgccatgcatggcggcggggttgaagcgccacgtggcgcccagatgggcggggccggctgacgtggaccccgccgtaggtggtggcggggtaggggttccccgccgcggatcatggcgggttcgcaccccgccccgcatcgtggcgggttaccccctgtcagaagaaaaccggggcccgacccgcacattgcccacaccgtaacgctgaccccgcgcacccgccgccccgctgccgtgcccgcgccgccgccccgccctctccgcgcccgcgccgccgccgcgctcgcgcccaaccccgcccccgcgcctgccgcgcacgtcctcccgcgccgagcacgccctcacgcggacgcccccagcccccggccgcgcgccgcggccgcgtcgctctgggcgcacggccgcgtcccgcccccgcggcgcgccgccgctgccgcgcacggcctccgcgccccgctgccgcgccgcggcacgccgctgctgcccggcctccgcgccctgccccatccggccgccttgttgtgaaggtattttttttatttaaactcatattattacatatttagtattattattttacattttgtattagttatttttattagttaaagttaaactttgtttagatattagttttatagtaattagtttttagttaatattattatttaatattagtagcgttatttagtgattagtgattagttattattatttaataatgtttagtgtgtttacatataagttacctagtaattagtaattagatattaagatttagtgattagtaattaataattagttattagttactattattatatagtaatttagttagtaaaatagttaataaattcggtagtactgtacttagttattttgtatttattagtaccatattggttagttgatgaggaatgtacttaaatcgttttgtagatggattacggtgtaagagttttttacggaggaagtgttaggagagaggattgtcagtttgaggatatgtctgaggatttggaatggtttga

SEQ ID NO: 94 - DNA, 4626 bp, Sorghum bicolor
  source 1..4626: mol_type = unassigned DNA
atgctcgtcggcacgctcggcgcgctcgtccacggctgctcgctccccgtcttcctccgcttcttcgccgacctcgtcgactccttcggctcccacgccaacgacccggacaccatggtccgcctcgtcgtcaagtacgccttctacttcctcgtcgtcggagccgcaatctgggcgtcctcatgggcagagatctcctgctggatgtggaccggcgagcggcagtcgacgcggatgcggatccggtacctggacgcggcgctgcggcaggacgtgtccttcttcgacaccgacgtgcgcgcctcggacgtcatctacgccatcaacgcggacgccgtggtggtgcaggacgccatcagcgagaagctgggcaacctcatccactacatggccaccttcgtggcgggcttcgtcgtgggcttcaccgccgcctggcagctggcgctcgtcacgctcgccgtcgtgccgctcatcgccgtcatcggggggctcagcgccgccgcgctcgccaagctctcctccaggagccaggacgcgctgtcgggcgccagcggcatcgcggagcaggcgctcgcgcagatacggatcgtgcaggccttcgtcggcgaggagcgcgaaatgcgggcgtactcggcggcgctggccgtcgcgcagaagatcggctaccgcagcggcttcgccaaggggctcggcctcggcggcacctacttcaccgtcttctgctgctacggcctcctgctctggtacggcggacacctcgtccgcggcaaccacaccaacggagggctcgccatcgccaccatgttctccgtcatgatcggcgggctggccctcgggcagtcggcgccgagcatggccgcgttcgccaaggcgcgcgtggcggccgccaagatcttccgcatcatcgaccacaggccgggcatctcctcgcgggacggcgaggacggcggcggcgtggagctggagtcggtgacggggcgggtggagatgaggggcgtggacttcgcgtacccgtcgcggccggacgtccccatcctgcgcggcttctcgctcagcgtgcccgccggcaagaccatcgcgctggtgggcagctccggctccgggaagagcacggtggtgtcgctcctcgagaggttctacgaccccagcgcagggcaaatcttgctggacgggcatgatctcaagtcgctgaagctccggtggctccggcagcagattggtctggtgagccaggagccgacgctgttcgcgacgagcatcaaggagaacctgctgctggggcgggacagtcagagtgcgacgcaggccgagatggaggaggccgccagggtggccaacgcgcactccttcatcgtcaagctccccgacggctacgacacgcaggttggggagcgcggcctgcagctctccggcgggcagaagcagcgcatcgccatcgcccgcgccatgctcaagaaccctgccatcctgctgctggacgaggctaccagcgcgctcgactccgagtcggagaagctcgtgcaggaggcgctggaccgcttcatgatcgggcgcaccaccctggtgatcgcgcacaggctgtccaccatccgcaaggccgacgtcgtggccgtgctgcagggcggcgccgtctccgagatgggcacccacgacgagctcatggccaagggcgagaacggcacctacgccaagctgatccgcatgcaggagcaggcgcacgaggcggcgctcgtcaacgcccgccgcagcagcgccaggccctccagcgcccgcaactccgtcagctcgcccatcatgacgcgcaactcctcctacggccgctccccctactcccgccgcctctccgacttctccacctccgacttcaccctctccatccacgacccgcaccaccaccaccggacgatggccgacaagcagctcgcgttccgcgccggcgccagctccttcctccgcctcgccaggatgaactcgcccgagtgggcctacgcgctcgtcggctccctgggctccatggtctgcggctccttcagcgccatcttcgcctacatcctcagcgccgtgctcagcgtctactacgcgccggaccctcgctacatgaagcgcgagatcgccaagtactgctacctgctcatcggcatgtcctccgcggcgctgctgttcaacacggtgcagcacgtgttctgggacacggtcggcgagaacctcacgaagcgtgtgcgcgagaagatgttcgccgccgtgctccgcaacgagatcgcctggttcgacgccgacgagaacgccagcgcgcgcgtcgccgccaggctcgcgctcgacgcccagaacgtgcgctccgccatcggggaccgtatctccgtcatcgtccagaactcggcgctcatgctcgtcgcctgcaccgcgggcttcgtcctccagtggcgcctcgcgctcgtgctcctcgccgtcttcccgctcgtcgtcggcgccaccgtcctgcagaagatgttcatgaagggcttctcgggggacctggaggccgcgcacgccagggccacgcagatcgcgggcgaggccgtcgccaacctgcgcaccgtggcggcgttcaacgcggagcgcaagatcacggggctcttcgaggccaaccttcgcggcccgctccggcgctgcttctggaaggggcagatcgccgggagcggctacggcgtggcgcagttcctgctgtacgcgtcctacgcgctggggctctggtacgccgcgtggctagtgaagcacggcgtctccgacttctcgcgcaccatccgcgtgttcatggtgctcatggtgtccgccaacggcgccgccgagacgctgacgctggcgccggactttgtcaagggcgggcgcgcgatgcggtccgtgttcgagaccatcgaccggaaaacggaggtggagcccgacgacgtggacgcggcgccggtgccggagcggcccaagggcgaggtggagctgaagcacgtggacttctcgtacccgtcgcggccggacatccaggtgttccgcgacctgagcctccgggcgcgcgccgggaagacgctggcgctggtgggtccgagcgggtgcggcaagagctcggtgctggcgctggtgcagcggttctacgagcccacgtccgggcgcgtgctcctggacggcaaggacgtgcgcaagtacaacctgcgggcgctgcggcgcgtggtggcggtggtgccgcaggagccgttcctgttcgcggcgagcatccacgacaacatcgcgtacgggcgcgagggcgcgacggaggcggaggtggtggaggcggcgacgcaggcgaacgcgcaccggttcatctcggcgctgccggagggctacgggacgcaggtgggcgagcgcggggtgcagctgtcgggcgggcagcggcagcggatcgcgatcgcgcgcgcgctggtgaagcaggcggccatcatgctgctggacgaggcgaccagcgcgctggacgccgagtcggagcggtggctcttcgaggccaaccttcgcggcccgctccggcgctgcttctggaaggggcagatcgccgggagcggctacggcgtggcgcagttcctgctgtacgcgtcctacgcgctggggctctggtacgccgcgtggctagtgaagcacggcgtctccgacttctcgcgcaccatccgcgtgttcatggtgctcatggtgtccgccaacggcgccgccgagacgctgacgctggcgccggactttgtcaagggcgggcgcgcgatgcggtccgtgttcgagaccatcgaccggaaaacggaggtggagcccgacgacgtggacgcggcgccggtgccggagcggcccaagggcgaggtggagctgaagcacgtggacttctcgtacccgtcgcggccggacatccaggtgttccgcgacctgagcctccgggcgcgcgccgggaagacgctggcgctggtgggtccgagcgggtgcggcaagagctcggtgctggcgctggtgcagcggttctacgagcccacgtccgggcgcgtgctcctggacggcaaggacgtgcgcaagtacaacctgcgggcgctgcggcgcgtggtggcggtggtgccgcaggagccgttcctgttcgcggcgagcatccacgacaacatcgcgtacgggcgcgagggcgcgacggaggcggaggtggtggaggcggcgacgcaggcgaacgcgcaccggttcatctcggcgctgccggagggctacgggacgcaggtgggcgagcgcggggtgcagctgtcgggcgggcagcggcagcggatcgcgatcgcgcgcgcgctggtgaagcaggcggccatcatgctgctggacgaggcgaccagcgcgctggacgccgagtcggagcggtgcgtgcaggaggcgctggagcgcgccgggaacggccgcaccaccatcgtggtggcgcaccggctggccacggtgcggaacgcgcacaccatcgccgtgatcgacgacggcaaggtggtggagcaagggtcgcactcgcacctgctcaagcaccatcccgacgggtgctacgcgcggatgctgcagctgcagcggctgacaggcggtgccgcgcccgggccgccgccgtcgtcgtccaacggggccgccgcgtag

SEQ ID NO: 95 - protein, 1402 aa, Sorghum bicolor
MSTNDPDEIRARVVVLGAPHADDDAGDEWARPELEAFHLPSPAHQPPGFHLAAGHQPEAAAEQPTTLPAARRTSDTSTAAGAAPPSPSPPPPPAPLEMDQPPNAKPASSSAAAAGANDNKKPTPPAALRDLFRFADGLDCALMLVGTLGALVHGCSLPVFLRFFADLVDSFGSHANDPDTMVRLVVKYAFYFLVVGAAIWASSWAEISCWMWTGERQSTRMRIRYLDAALRQDVSFFDTDVRTSDVIYAINADAVVGAGRHQREAGQPHPLHGHLRGGLRRGLHRRLAAGARHARRRAAHRRHRGAQRRRARQALLQEPGRAVGRQRHRGAGARADTDRAGLRRRGARNAGVLGGVGRRAEDRLPQRLRQGARPRRHLLHRLLLLRPPALVRRTPRPRNHTNGGLAIATMFSVMIGGLALGQSAPSMAAFAKARVAAAKIFRIIDHRPGISSRDGEDGGGVELESVTGRVEMRGVDFAYPSRPDVPILRGFSLSVPAGKTIALVGSSGSGKSTVVSLLERFYDPSAGQILLDGHDLKSLKLRWLRQQIGLVSQEPTLFATSIKENLLLGRDSQSATQAEMEEAARVANAHSFIVKLPDGYDTQVGERGLQLSGGQKQRIAIARAMLKNPAILLLDEATSALDSESEKLVQEALDRFMIGRTTLVIAHRMSTIRKADVVAVLQGGPVSEMGAHDELMAKGENGTYAKFIRMQEQAHEAAFVNARRSSARPSSARNSVSSPIMTRNSSYGRSPYSRRLSDFSTSDFTLSIHDPHHHHRTMADKQLAFRAGASSFLRLARMNSPEWAYALVGSLGSMVCGSFSAIFAYILSAVLSVYYAPDPRYMKREIAKYCYLLIGMSSAALLFNTVQHVFWDTVGENLTKRVREKMFAAVLRNEIAWFDADENASARVAARLALDAQNVRSAIGDRISVIVQNSALMLVACTAGFVLQWRLALVLLAVFPLVVAATVLQKMFMKGFSGDLEAAHARATQIAGEAVANLRTVAAFNAERKITGLFEANLRGPLRRCFWKGQIAGSGYGVAQFLLYASYALGLWYAAWLVKHGVSDFSRTIRVFMVLMVSANGAAETLTLAPDFVKGGRAMRSVFETIDRKTEVEPDDVDAAPVPERPKGEVELKHVDFSYPSRPDIQVFRDLSLRARAGKTLALVGPSGCGKSSVLALVQRFYEPTSGRVLLDGKDVRKYNLRALRRVVAVAPQEPFLFAASIHDNIAYGREGATEAEVVEAATQANAHRFIAALPEGYGTQVGERGVQLSGGQRQRIAIARALVKQAAIVLLDEATSALDAESERCVQEALERAGSGRTTIVVAHRLATVRGAHTIAVIDDGKVAEQGSHSHLLKHHPDGCYARMLQLQRLTGGCRARAAAVVVQRGRRVGWMDGSWMSLVP

SEQ ID NO: 96 - DNA, 20 bp, Sorghum bicolor
  source 1..20: mol_type = unassigned DNA
tgtctgtaaatcgcgagacg

SEQ ID NO: 97 - DNA, 22 bp, Sorghum bicolor
  source 1..22: mol_type = unassigned DNA
tcgtgacgacactgatagaacg

SEQ ID NO: 98 - DNA, 26 bp, Sorghum bicolor
  source 1..26: mol_type = unassigned DNA
ccttgtaagtagacgatggctagctc

SEQ ID NO: 99 - DNA, 24 bp, Sorghum bicolor
  source 1..24: mol_type = unassigned DNA
tgaatgattgctactatcggtgag

SEQ ID NO: 100 - protein, 319 aa, Oryza sativa
MMASCRRGGGGDVDRIKGPWSPEEDEALQRLVGRHGARNWSLISKSIPGRSGKSCRLRWCNQLSPQVEHRPFTPEEDDTILRAHARFGNKWATIARLLAGRTDNAIKNHWNSTLKRKHHSSLLADDLRPLKRTTSDGHPTLSSAAAPGSPSGSDLSDSSHHSLPSQMPSSPPHLLLPQHVYRPVARAGGVVVPPPPPPPPPATSLSLSLSLPGLDHPHPDPSTPSEPAVQLQPPPPSQMPPPTPSCVRQEPPQMPFQLQPPPPPRPSAPFSAEFLAMMQEMIRIEVRNYMSGSAAVDPRSSPDNGVRAASRIMGMAKIE

SEQ ID NO: 101 - protein, 338 aa, Oryza sativa
MAHEMMGGFFGHPPPPPATAAVGEEEDEVVEETEEGGHGGGVQGKLCARGHWRPAEDAKLKDLVAQYGPQNWNLIAEKLDGRSGKSCRLRWFNQLDPRINRRAFTEEEEERLMAAHRAYGNKWALIARLFPGRTDNAVKNHWHVLMARRHREQSGAFRRRKPSSSSASPAPAPAPPPPPQPVVALHHHHHRYSQQYSGYSGAAESDESASTCTTDLSLSSGSAAAAAAAAAAANIPCCFYQSTPRASSSSTAACRAPRVAAAADTVAFFPGAGYDFAAAPHAMAPAAASTFAPSARSAFSAPAGRGEPPGAVDQRGGAQATTDSHTIPFFDFLGVGAT

SEQ ID NO: 102 - protein, 267 aa, Oryza sativa
MASSSTTNTSDGAGKPASSSSSACPRGHWRPGEDEKLRQLVEKYGPQNWNSIAEKLEGRSGKSCRLRWFNQLDPRINKRPFTEEEEERLLAAHRHHGNKWALIARHFPGRTDNAVKNHWHVVRARRSRERSRLLARAAAAAAHPPPFSSYASQLDFSGGGASSGARNSSLCFGFGMINRSSSSSSSPAAAPFLIKSFNGTSYGTLLPATTSMAAAAQPVSTITFSSTPMRETLELMDAGGHENHGDVDGGGDKRKGVPYFDFLGVGV

SEQ ID NO: 103 - protein, 245 aa, Oryza sativa
MCTRGHWRPSEDEKLKELVARYGPHNWNAIAEKLQGRSGKSCRLRWFNQLDPRINRSPFTEEEEELLLASHRAHGNRWAVIARLFPGRTDNAVKNHWHVIMARRCRERMRLSNRRGGAAAAGAAKGDESPARISNGEKTATRPPATNGSGMAMASLLDKYRRECGAAGLFAIGRHHNSKEDYCSSTNEDTSKSVEFYDFLQVNASSSDTKCGSSIEEQEDNRDDDQAEGQVQLIDFMEVGTTSRQ

SEQ ID NO: 104 - protein, 303 aa, Sorghum bicolor
MAAFFGAAPPPPPPPGLASFHHHEEEEQQHHKDAAEEVYHVDDVDEGGAGQGKLCARGHWRPAEDAKLKELVAQYGPQNWNLIAERLDGRSGKSCRLRWFNQLDPRINRRAFSEEEEERLLAAHRAYGNKWALIARLFPGRTDNAVKNHWHVLMARKQREQTGALRRRKPSSSSSSSPGPAPTPHFAPVVVLHHHHYAGSPPMPLHATAGAYTTAAAAADTRAHSGGESDETASTCTTDLSLGSAAAPCFYQSGYDVVPRAAAFAPSARSAFSAPSATARHGEARSDDKVSLPFFDFLGVGAA

SEQ ID NO: 105 - protein, 282 aa, Sorghum bicolor
MAHEMACFLGPAPPVPALSPFHEGQAESHGHGHGSSRSGHSRGHWRPAEDAKLKDLVAQYGPQNWNLIANKLHGRSGKSCRLRWFNQLDPRLNRRPFSEEEEERLLAAHRAYGNKWALIARLFPGRTDNAVKNHWHVLMARKQREHLSGSGGGAPRRRKPSSSSSASSSSSSSAVDVVVRHQHASSPLPFREAAAGAVTTRARAYSDDGESDESVSVSTSGATDLSLGSVSGTTCLLGSLPLPHCVLGSSVPSPARHRAAASDDGRGKLALPFFDFLGVGAT

SEQ ID NO: 106 - protein, 401 aa, Sorghum bicolor; identical to SEQ ID NO: 92

SEQ ID NO: 107 - protein, 442 aa, Sorghum bicolor
MPCSSAAPTWLLRVASAADQASSSSSSKGGGRVLTAGTTGTTMDTAATAAAAGCGGNGGGGGGSNAADLQESSSSGQSRLAARGHWRPAEDAKLRELVALYGPQNWNLIAEKLDGRSGKSCRLRWFNQLDPRISKRPFSDEEEERLMAAHRFYGNKWAMIARLFPGRTDNAVKNHWHVIMARKYREQSTAYRRRKLNQAVQRKLEASAAAVATMPPAAGSTGDVVGAALGHHHHQLLAAAAAAAHDAAYGFAAADPYGAFGFRQYYPFPPASAEDTPPPPPPPFCLFPATSYFNLSSVAITPLCHATRSRCAKICLSFDPWHIYIYIYTGPSSAAALHADSRRLPWPSSSSSDAAAAAAGGGRYGEPQQQLLLPVVHGGSWIDGVGVAVAGGHHEAQFVLGNNGGAFEGTTRQQGAAAGAHFEAAAAAPPPAFIDFLGVGAT

SEQ ID NO: 108 - DNA, 1329 bp, Sorghum bicolor
  source 1..1329: mol_type = unassigned DNA
atgccgtgctcgtcggcggccccgacgtggctgctgcgggtggcgtcggcggccgaccaggcctcgtcctcgtcctcgtccaagggcggcggccgcgtgctcaccgccggcaccactggcaccaccatggacacggccgccaccgctgccgccgccggctgtggcggcaacggcggcgggggaggcggcagtaatgccgccgacctccaggagagcagcagcagcgggcagtcccggctcgcggcgcgcggccactggcgccccgccgaggacgccaagctccgcgagctcgtcgcgctctacggtccccagaactggaacctcatcgccgagaagctcgacggcagatccgggaagagctgccgcctccggtggttcaaccagctggacccgcggatcagcaagcggcccttcagcgacgaggaagaagagcggctgatggcggcgcaccgcttctacggcaacaagtgggcgatgatcgcgcgcctgttcccggggcgcacggacaacgccgtcaagaaccactggcacgtcatcatggcgcgcaagtaccgcgagcagtccacggcgtaccgccgccgcaagctcaaccaggcagtccagcggaagctcgaggcctccgccgcggcggtcgcaacaatgccgccggccgcgggcagcacgggagacgtcgtcggcgccgccctcggccaccaccaccaccaactcctggcggccgccgccgccgccgcccacgacgcggcctacggcttcgccgcggcggacccctacggcgccttcggcttccgccaatactacccgttcccgccagcttcggccgaggacacgccgccgccgccgccgcctcccttctgcttgttccctgccactagctacttcaatttgtcatcagtcgctataacaccactgtgtcacgccacgcgatcgaggtgtgcaaaaatctgcttgtcttttgatccatggcatatatatatatatatatatacagggcccagcagcgcggcggcgcttcacgccgacagcaggcgccttccctggccgtcgtcgtcgtcgtcggatgctgccgctgccgccgccggtggcggcaggtacggggagccgcagcagcagctcctgctgcccgttgttcacggtggcagctggatcgacggcgtcggcgtggccgtggccggcggtcaccacgaggcgcagttcgtcttgggcaacaacgggggagcctttgaagggaccacaagacagcagggcgccgccgccggcgctcactttgaagctgccgcggcggcgccgccgccagcgttcatagatttcctcggtgtcggagccacatga

SEQ ID NO: 109 - DNA, 1209 bp, Sorghum bicolor
  source 1..1209: mol_type = unassigned DNA
atgccgtgctcgtcggcggccccgacgtggctgctgcgggtggcgtcggcggccgaccaggcctcgtcctcgtcctcgtccaagggcggcggccgcgtgctcaccgccggcaccactggcaccaccatggacacggccgccaccgctgccgccgccggctgtggcggtggcgcctgcggcgggggaggcggcagtaatgccgccgacctccaggagagcagcagcagcgggcagtcccggctcgcggcgcgcggccactggcgccccgccgaggacgccaagctccgcgagctcgtcgcgctctacggtccccagaactggaacctcatcgccgagaagctcgacggcagatccgggaagagctgccgcctccggtggttcaaccagctggacccgcggatcagcaagcggcccttcagcgacgaggaagaagagcggctgatggcggcgcaccgcttctacggcaacaagtgggcgatgatcgcgcgcctgttcccggggcgcacggacaacgccgtcaagaaccactggcacgtcatcatggcgcgcaagtaccgcgagcagtccacggcgtaccgccgccgcaagctcaaccaggcagtccagcggaagctcgaggcctccgccgcggcggtcgcaacaatgccgccggccgcgggcagcacgggagacgtcgtcggcgccgccctcggccaccaccaccaccaactcctggcggccgccgccgccgccgcccacgacgcggcctacggcttcgccgcggcggacccctacggcgccttcggcttccgccaatactacccgttcccgccagcttcggccgaggacacgccgccgccgccgccgcctcccttctgcttgttccctgggcccagcagcgcggcggcgcttcacgccgacagcaggcgccttccctggccgtcgtcgtcgtcgtcggatgctgccgctgccgccgccggtggcggcaggtacggggagccgcagcagcagctcctgctgcccgttgttcacggtggcagctggatcgacggcgtcggcgtggccgtggccggcggtcaccacgaggcgcagttcgtcttgggcaacaacgggggagcctttgaagggaccacaagacagcagggcgccgccgccggcgctcactttgaagctgccgcggcggcgccgccgccagcgttcatagatttcctcggtgtcggagccacatga

SEQ ID NO: 110 - DNA, 1418 bp, Sorghum bicolor
  misc_feature 1042: note = n is a, t, c, or g
  misc_feature 1053: note = n is a, t, c, or g
  source 1..1418: mol_type = unassigned DNA
atgccgtgctcgtcggcggccctgacgtggctgctgcgggtggcgtcggcggccgaccaggcctcgtcctcgtcctcgtccaagggcggcggccgcgagctcaccgccggcaccactggcaccaccatggacacggccgccaccgctgccgccgccggctgtggcggtggcgcctgcggcgggggaggcggcagtaatgccgccgacctccaggagagcggcagcagcgggcagtcccggctcgcggcgcgcggccactggcgccccgccgaggacgccaagctccgcgagctcgtcgcgctctacggtccccagaactggaacctcatcgccgagaagctcgacggcagatccgggaagagctgccgcctccggtggttcaaccagctggacccgcggatcagcaagcggcccttcagcgacgaggaggaagagcggctgatggcggcgcaccgcttctacggcaacaagtgggcgatgatcgcgcgcctgttcccggggcgcacggacaacgccgtcaagaaccactggcacgtcatcatggcgcgcaagtaccgcgagcagtccacggcgtaccgccgccgcaagctcaaccaggcagtccagcggaagctcgaggcctccgccgcggcggtcgcaacaatgccgccggccgcgggcagcacgggagacgtcgtcggcgccgccctcggccaccaccaccaccaactcctggcggccgccgccgccgccgcccacgacgcggcctacggcttcgccgcggcggacccctacggcgccttcggcttccgccaatactacccgttcccgccagcttcggccgaggacacgccgccgccgccgccgcctcccttctgcttgttccctgaaccatatgttgaggctccatttgctcccgttcaacctcaccatgcgcgcgtggggaagatgagccagcgccaacggacgggatgtgaatcgaggattgcgttaactgcaggctgcgcgtggggaagatgagccagcgttaattgtaggctgagcatgggttgcgttaactgcnggccggggggngcttgcatgcagccatgcagagggggcccagcagcgcggcggcgcttcacgccgacagcaggcgccttccctggccgtcgtcgtcgtcgtcggatgctgccgctgccgccgccggtggcggcaggtacggggagccgcagcagcagctcctgctgcccgttgttcacggtggcagctggatcgacggcgtcggcgtggccgtggccggcggtcaccacgaggcgcagttcgtcttgggcaacaacgggggagcctttgaagggaccacaagacagcagggcgccgccgccggcgctcactttgaagctgccgcggcggcgccgccgccagcgttcatagatttcctcggtgtcggagccacatga

SEQ ID NO: 111 - DNA, 143 bp, Zea mays
  source 1..143: mol_type = unassigned DNA
gagataattgtcattatagacgaagagcggacgggattcgacgaaatggaggcgatggcgttggcttctctgttctggaaacgcagacgacagccaaacgccaaaacggaaaggagacagcgcttggagctgtgtaaacaggt

(19) United States
(12) Patent Application Publication
(10) Pub. No.: US 2025/0236882 A1
(43) Pub. Date: Jul. 24, 2025

(54) COMPOSITIONS AND METHODS FOR STATURE MODIFICATION IN PLANTS

(71) Applicant: PIONEER HI-BRED INTERNATIONAL, INC., JOHNSTON, IA (US)

(72) Inventors: HUIRONG GAO, JOHNSTON, IA (US); ROBERT B. MEELEY, DES MOINES, IA (US); SHUPING JIAO, JOHNSTON, IA (US); SABRINA HUMBERT, JOHNSTON, IA (US); DILBAG S. MULTANI, URBANDALE, IA (US); XIAOMU NIU, JOHNSTON, IA (US); JOAN MARIE PHILLIPS, PROLE, IA (US)

(73) Assignee: PIONEER HI-BRED INTERNATIONAL, INC., JOHNSTON, IA (US)

(21) Appl. No.: 19/028,415

(22) Filed: Jan. 17, 2025

Related U.S. Application Data
(63) Continuation of application No. 16/634,275, filed on Jan. 27, 2020.
(60) Provisional application No. 62/558,619, filed on Sep. 14, 2017.

(51) Int. Cl. C12N 15/82

(52) U.S. Cl. CPC C12N 15/8297; C12N 15/8213; C12N 15/8261

(57) ABSTRACT

Methods and compositions to modulate plant stature including plant height, car height, planting density and other agronomic characteristics are disclosed. The disclosure further discloses compositions, polynucleotide constructs, transformed host cells, plants and seeds exhibiting altered stature characteristics or produce plants that exhibit altered stature parameters.

### CROSS-REFERENCE TO RELATED APPLICATIONS

This application is a continuation application of U.S. patent application Ser. No. 16/634,275, filed Jan. 27, 2020, which is a National Stage application of International Patent Application number PCT/US18/44498, filed Jul. 31, 2018, which claims the benefit of priority to U.S. Provisional Application No. 62/558,619 filed Sep. 14, 2017, the disclosures of each of which is are incorporated herein by reference in their entireties.

### REFERENCE TO SEQUENCE LISTING SUBMITTED ELECTRONICALLY

The official copy of the sequence listing is submitted electronically via Patent Center as an XML formatted sequence listing with a file named 106531-US-CON-1.xml created on Jan. 14, 2025, and having a size of 204,889 bytes and is filed concurrently with the specification. The sequence listing comprised in this XML formatted document is part of the specification and is herein incorporated by reference in its entirety. The sequence descriptions (Table 1) and sequence listing attached hereto comply with the rules governing nucleotide and amino acid sequence disclosures in patent applications as set forth in 37 C.F.R. §§ 1.831-1.835.

### FIELD

This disclosure relates to compositions and methods of modifying stature in plants, including height reduction.

### BACKGROUND

Recent advances in plant genetic engineering have opened new doors to engineer plants to have improved characteristics or traits, such as stature, height and other architecture. Plant height is a desirable trait in crop breeding for a variety of crops of commercial interest. Dwarf stature has been used to improve yield and lodging resistance in crop plants, e.g., use of dwarf mutants in wheat and rice that increased harvest index. Height adaptations increase harvest index, favorably partition carbon and nutrients between grain and none-grain biomass, enhance fertilizer use, water use efficiency and plays a role in increasing planting density.

### SUMMARY

A method of reducing plant height, the method comprising introducing one or more nucleotide modifications through a targeted DNA break at a genomic locus of a plant, wherein the genomic locus comprises a polynucleotide involved in a biological process selected from the group consisting of gibberellic acid biosynthesis, gibberellic acid signaling, auxin transport, auxin signaling, brassinosteroid biosynthesis or signaling, brachytic 2 (Br2), MYB transcription factor expression or activity, and wherein the plant height is reduced compared to a control plant not comprising the one or more introduced genetic modifications.

In an embodiment, the reduction in plant height is in the absence of a substantial reduction in grain yield measure per plant or as a population of plants per unit area.

In an embodiment, the genetic modifications target more than one distinct genomic loci that are involved in plant height reduction. In an embodiment, the plant height is reduced by about 5% to about 30% compared to the control plant. In an embodiment, the plant comprises an average leaf length to width ratio reduced at V6-V8 growth stages. In an embodiment, the plant height reduction does not substantially affect flowering time. In an embodiment, the flowering time does not change by more than about 5-10 CRM or plus or minus 10% GDU or 125-250 GDU, compared to a control plant not comprising the modifications, wherein 25 GDU is equivalent to about 1 day and 1 CRM is about 1 day. In an embodiment, the plant height reduction does not substantially alter root architecture of the plant or does not significantly increase root lodging, compared to a control plant not comprising the modifications. In an embodiment, the plant is substantially tolerant to lodging as measured at a single plant level or at an increased planting density, compared to a control plant or control population of plants. In an embodiment, the plant comprises up to about 10% less number of leaves compared to the control plant. In an embodiment, the plant is maize and the plant height reduction is characterized by the shortening of distance between one or more internodes that are present above or below a female reproductive part of the maize plant. Modified maize plants, whose average internode lengths are reduced compared to the wild-type plants are provided. For example, average internode length (2^{nd} internode length and/or 4^{th} internode length relative to the position of the car) that is at least 5%, 10%, 15%, 20%, 25%, 30%, 35%, 40%, 45%, 50%, 55%, 60%, 65%, 70%, 75% less than the same or average internode length of a wild-type or control plant are provided. The 2^{nd} internode” refers to the second internode below the ear of the corn plant, likewise, the “4^{th} internode” refers to the fourth internode below the car of the corn plant.

Plants are provided that have (i) a plant height that is at least 5%, 10%, 15%, 20%, 25%, 30%, 35%, 40%, 45%, 50%, 55%, 60%, 65%, 70%, 75% less than the height of a wild-type or control plant, and/or (ii) a stem or stalk diameter that is at least 5%, 5%, 10%, 15%, 20%, 25%, 30%, 35%, 40%, 45%, 50%, 55%, 60%, 65%, 70%, 75%, 80% greater than the stem diameter of the wild-type or control plant.

In an embodiment, one or more of the following agronomic characteristics of the plant is increased or reduced: harvest index of the plant is increased; leaf area is increased; leaf number above the car is reduced; ratio of the plant car height over the plant height is increased; and the yield is increased at higher planting density, as compared to a control plant not comprising the mutations. In an embodiment, the plant is selected from the group consisting of maize, sorghum, rice, wheat and barley.

In an embodiment, the plant is maize, and the maize plant comprises an car, wherein the car height as measured to the maize plant height is substantially similar or slightly reduced to the car height measured relative to the control plant height. In an embodiment, the modifications target the genomic locus such that more than one genetic modifications are present within (a) the same coding region; (b) non-coding region; (c) regulatory sequence; or (d) untranslated region, of an endogenous polynucleotide encoding a polypeptide that is involved in plant height.

In an embodiment, the gibberellic acid biosynthesis genomic locus comprises a polynucleotide that encodes a polypeptide comprising an amino acid sequence that is at least 95% identical to a sequence selected from the group consisting of SEQ ID NOS: 75-76. In an embodiment, the auxin transport or auxin signaling genomic locus comprises a polynucleotide that encodes a polypeptide comprising an amino acid sequence that is at least 95% identical to a maize polypeptide sequence involved in auxin signaling. In an embodiment, the MYB transcription factor genomic locus comprises a polynucleotide that encodes a polypeptide comprising an amino acid sequence that is at least 95% identical to a sequence selected from the group consisting of SEQ ID NOS: 1-9.

In an embodiment, the MYB transcription factor genomic locus comprises an edit in a polynucleotide that encodes a polypeptide comprising an amino acid sequence that is at least 95% identical to a sequence selected from the group consisting of SEQ ID NOS: 1-9, such that the edit results in one or more of the following:
(a) reduced expression of a polynucleotide encoding the MYB transcription factor;
(b) reduced transcriptional activity of the MYB transcription factor;
(c) generation of one or more alternative spliced transcripts of a polynucleotide encoding the MYB transcription factor;
(d) deletion of one or more DNA binding domains of the MYB transcription factor;
(e) frameshift mutation in one or more exons of a polynucleotide encoding the MYB transcription factor;
(f) deletion of a substantial portion of the polynucleotide encoding the MYB transcription factor or deletion of the polynucleotide encoding the full-length MYB transcription factor;
(g) repression of an enhancer motif present within a regulatory region encoding the MYB transcription factor;
(h) modification of one or more nucleotides or deletion of a regulatory element operably linked to the expression of the polynucleotide encoding the MYB transcription factor, wherein the regulatory element is present within a promoter, intron, 3′UTR, terminator or a combination thereof.

In an embodiment, the Br2 genomic locus comprises an edit in a polynucleotide that encodes a Br2 polypeptide comprising an amino acid sequence that is at least 95% identical to SEQ ID NO: 43, such that the edit results in results in
(a) reduced expression of a polynucleotide encoding the Br2 polypeptide;
(b) reduced activity of the Br2 polypeptide;
(c) generation of one or more alternative spliced transcripts of a polynucleotide encoding the Br2 polypeptide;
(d) deletion of one or more domains of the Br2 polypeptide;
(e) frameshift mutation in one or more exons of a polynucleotide encoding the Br2 polypeptide;
(f) deletion of a substantial portion of the polynucleotide encoding the Br2 polypeptide or deletion of the polynucleotide encoding the Br2 polypeptide;
(g) repression of an enhancer motif present within a regulatory region encoding the Br2 polypeptide;
(h) modification of one or more nucleotides or deletion of a regulatory element operably linked to the expression of the polynucleotide encoding the Br2 polypeptide, wherein the regulatory element is present within a promoter, intron, 3′UTR, terminator or a combination thereof.

In an embodiment, the double strand or single strand break is induced by using a guide RNA that corresponds to a target sequence selected from the group consisting of SEQ ID NOS: 22-42, 46-71.

In an embodiment, the gibberellic acid biosynthesis or signaling pathway is modulated by one or more introduced nucleotide changes at D8 genetic loci selected from the group consisting of:
(a) reduced expression of a polynucleotide encoding the D8 polypeptide;
(b) reduced activity of the D8 polypeptide;
(c) generation of one or more alternative spliced transcripts of a polynucleotide encoding the D8 polypeptide;
(d) deletion of one or more domains of the D8 polypeptide;
(e) frameshift mutation in one or more exons of a polynucleotide encoding the D8 polypeptide;
(f) deletion of a substantial portion of the polynucleotide encoding the D8 polypeptide or deletion of the polynucleotide encoding the Br2 polypeptide;
(g) repression of an enhancer motif present within a regulatory region encoding the D8 polypeptide;
(h) modification of one or more nucleotides or deletion of a regulatory element operably linked to the expression of the polynucleotide encoding the D8 polypeptide, wherein the regulatory element is present within a promoter, intron, 3′UTR, terminator or a combination thereof.

A method of identifying a Br1 allele (MYB transcription factor) in a population of plants, the method comprising isolating a polynucleotide of a genomic region, wherein the genomic region encodes a polypeptide that is at least 95% identical to the amino acid sequence selected from the group consisting of SEQ ID NOS: 1-9; sequencing the polynucleotide to identify one or more nucleotide variations; and identifying the Br1 allele based on a phenotype selected from the group consisting of: reduced plant height, ear height, reduced root lodging, reduced leaf width to length ratio, reduced shade avoidance, reduced leaf number above the ear, increased leaf area per plant, and a combination thereof when compared to a control plant. In an embodiment, the plant is maize, rice, wheat, barley, sorghum or cotton. In an embodiment, the plant is maize and the plant height is reduced by about 10% to about 30% compared to the control plant.

In an embodiment, the one or more Br1 alleles are introduced through a genome modification technique selected from the group consisting polynucleotide-guided endonuclease, CRISPR-Cas endonucleases, zinc finger nuclease, a transcription activator-like effector nuclease (TALEN), engineered site-specific meganucleases, or Argonaute.

A plant comprising a modified Br1 genomic locus, wherein the Br1 genomic locus comprises one or more mutations compared to a control plant and wherein the Br1 genomic locus encodes a polypeptide that is at least 95% identical to the amino acid sequence selected from the group consisting of SEQ ID NOS: 1-9. In an embodiment, the plant is semi-dwarf when compared to the control plant. In an embodiment, the plant is maize or sorghum.

A Br1 mutant maize plant comprising a polypeptide sequence that is at least 95% identical to SEQ ID NOS: 1-5 and wherein the mutant maize plant exhibits reduced plant height.

A recombinant DNA construct comprising a polynucleotide sequence comprising any of the nucleotide sequences set forth Table 1, operably linked to at least one heterologous nucleic acid sequence. In an embodiment, the plant cell includes the recombinant construct.

A guide RNA sequence that targets a genomic loci of a plant cell, wherein the genomic loci comprises a polynucleotide that encodes a polypeptide comprising an amino acid sequence that is at least 90% identical to a sequence selected from the group consisting of SEQ ID NOS: 1-9, 75-76. In an embodiment, the recombinant DNA construct expresses the guide RNA. In an embodiment, the plant cell includes the guide RNA.

A plant having stably incorporated into its genome the recombinant DNA construct disclosed herein. In an embodiment, the plant is a monocot plant. In an embodiment, the plant is maize, soybean, rice, wheat, sunflower, cotton, sorghum or canola. A seed produced by the plant disclosed herein.

In an embodiment, the plant further includes a heterologous nucleic acid sequence selected from the group consisting of: a reporter gene, a selection marker, a disease resistance gene, a herbicide resistance gene, an insect resistance gene; a gene involved in carbohydrate metabolism, a gene involved in fatty acid metabolism, a gene involved in amino acid metabolism, a gene involved in plant development, a gene involved in plant growth regulation, a gene involved in yield improvement, a gene involved in drought resistance, a gene involved in increasing nutrient utilization efficiency, a gene involved in cold resistance, a gene involved in heat resistance and a gene involved in salt resistance in plants.

A mutant maize plant that comprises an introduced genetic modification at more than one genomic loci, the genomic loci comprises a component involved in a biological pathway selected from the group consisting of gibberellic acid biosynthesis, gibberellic acid signaling, auxin transport, auxin signaling, brassinosteroid biosynthesis or signaling, Brachytic 2 (Br2), MYB transcription factor expression or activity, and a combination thereof, wherein the genetic modification is introduced by a site-specific endonuclease in vivo and wherein the mutant maize plant exhibit reduced plant height and or car height.

A sorghum plant, wherein the sorghum plant exhibits a shorter stature (e.g., semi dwarf) compared to a control plant (e.g., tall), wherein the sorghum plant comprises a mutation in a genomic locus, the genomic locus encodes a polypeptide comprising an amino acid sequence that is at least 90% identical to one of SEQ ID NOS: 6, 88, 104-105 (e.g., Dw5, orthologs and variants thereof) and the control plant does not comprise the mutation. In an aspect, the sorghum plant of claim 62, wherein the polypeptide comprises an amino acid sequence that is at least 95% identical to one of SEQ ID NOS: 6, 88, 104-105. In an aspect, the polypeptide comprises an amino acid sequence that is at least 98% identical to one of SEQ ID NOS: 6, 88, 104-105. In an aspect, the mutation is an insertion or deletion of one or more nucleotides in the genomic locus. In an aspect, the mutation results in a non-functional polypeptide. In an aspect, the mutation results in a significant reduction in the expression or activity of the polypeptide.

A sorghum seed produced from a sorghum plant that has modified at a genomic locus comprising Dw3 or a plant cell of the sorghum plant.

A population of sorghum plants comprising a genetic modification in a genomic locus represented by dwarf 3 (dw3), wherein at least 90% of the population of sorghum plants exhibit dwarf phenotype compared to a control population of sorghum plants not comprising the genetic modification. In an aspect, at least 95% of the population of sorghum plants exhibit dwarf phenotype. In an aspect, at least 99% of the population of sorghum plants exhibit dwarf phenotype.

In an aspect, the genetic modification results in a reduction of the dw3 dwarf allele from reverting to a wild type (tall) allele in a sorghum plant. In an aspect, the genetic modification is a deletion of one or more regions of the dw3 genomic locus. In an aspect, the dw3 gene is non-functional.

A method of reducing reversion frequency of a dwarf 3 (dw3) allele in a sorghum plant to a tall phenotype, the method comprising introducing a genetic modification at a genomic locus comprising the dw3 allele by a site-specific genome editing method and obtaining a modified sorghum plant that exhibits reduced reversion frequency of reversion to the tall phenotype when compared to a control sorghum plant not comprising the modification. In an aspect, the genome editing method comprises CRISPR-Cas endonuclease. In an aspect, the genome editing method comprises targeted base editing. In an aspect, the genome editing method comprises a method selected from the group comprising Zn finger nuclease, meganuclease, and TALEN. In an aspect, the genome editing method comprises CRISPR-Cas9 or Cpf1 endonuclease. In an aspect, the dw3 genomic locus encodes a polypeptide that is at least 90% identical to the full length sequence of SEQ ID NO: 95. In an aspect, the reversion frequency is less than about 15%, 10%, 5%, 1%, 0.5%, 0.1% or 0.05% compared to the control sorghum plant.

A modified semi-dwarf sorghum plant includes a modified Dw3 locus wherein the modified Dw3 locus does not comprise direct repeat in exon of the Dw3 gene, wherein the Dw3 gene encodes a polypeptide that is at least 95% identical to SEQ ID NO: 95 and the modified sorghum plant exhibits reduced reversion to wild-type (tall) phenotype. In an aspect, the modified Dw3 locus comprises a modification introduced by genome editing involving a site-directed guided endonuclease. In an aspect, the modified Dw3 locus comprises a deletion of the direct repeat in exon 5 of the Dw3 gene or deletion of a substantial portion of the Dw3 gene or deletion of the entire Dw3 coding region. In an aspect, the sorghum plant exhibits less than 15% reversion to wild-type (tall) phenotype. In an aspect, the modified sorghum plant exhibits less than 10% reversion to wild-type (tall) phenotype. In an aspect, the modified sorghum plant exhibits less than 5% reversion to wild-type (tall) phenotype.

A dwarf sorghum plant that exhibits a plant height reduction of about 25% to about 75% of a wild-type sorghum plant, wherein the dwarf plant comprises a double mutant comprising a dwarf dw3 allele and a dwarf dw5 allele, wherein the dw3 allele comprises a genomic modification resulting in less than 15% reversion to wild-type phenotype, when compared to a control sorghum plant not comprising the genomic modification. In an aspect, the genomic modification is introduced through CRISPR-Cas endonuclease. In an aspect, the reversion frequency of the double mutant sorghum plant is less than about 15%, 10%, 5%, 1%, 0.5%, 0.1% or 0.05% compared to the control sorghum plant.

A method of marker-assisted selection of a plant, the method includes performing a plurality of sequencing, polymerase chain reaction (PCR), probe hybridization reactions or a combination thereof on one or more samples obtained from a plant population and obtaining sequence information, probe hybridization data, amplified fragments or a combination thereof to determine genotypic variation at a genomic locus of brachytic 1 (Br1) or dwarf 5 (Dw5) and associating the genotypic information with the stature of the plant. In an aspect, the Br1 locus is characterized by encoded polypeptide comprising an amino sequence that is at least 90% identical to one of SEQ ID NOS: 1-9, 100-106. In an aspect, the Dw5 locus is characterized by encoded polypeptide comprising an amino sequence that is at least 90% identical to one of SEQ ID NOS: 6, 88, 104-105.

### BRIEF DESCRIPTION OF THE DRAWINGS AND SEQUENCE LISTING

The disclosure can be more fully understood from the following detailed description and the accompanying drawings and Sequence Listing that form a part of this application, which are incorporated herein by reference.

FIG. 1 shows plant height of the br1-CooP mutant in BC3F3 as compared to its WT-sib.

FIG. 2A shows plant height of br1-Mutag, a weak mutant allele in BC2F2 as compared to its WT-sib. The br1-Mutag allele was introgressed into an inbred line and characterized for plant and car heights. FIG. 2B shows car height of the br-Mutag mutant compared to its WT-sib at the BC2F2 generation. FIG. 2C shows a graph of average plant and car height of the br1-Mutag mutant plants compared to heterozygous sibs and WT sibs.

FIG. 3 shows cloning and gene structure of a candidate gene for br1 mutation is a diagrammatic representation of gene structure of br1-candidate gene Transcription Regulator HTH, MYB-type DNA binding protein (dpzm01g068810). The br1-candidate gene has three exons (empty rectangles) and two introns (line). Both forward and reverse gene specific primers (GSP) are marked with arrow showing their directions. The br1-Mutag weak allele has insertion of a Mutator element in intron1 (triangle) 15 bp from the intron1 and exon2 junction. The br1-CooP mutant allele has insertion of a retro-transposon element of 2.8 kb size present in exon3 (triangle). Solid rectangles represent 5′ and 3′ untranslated regions (UTR) of the gene.

FIG. 4 shows average expression of ZmBr1 (dpzm01g068810.1.1) gene in different corn tissues compiled from Lynx MPSS databases. Gene expression of Br1 based on combined databases of Illumina WgT was also performed.

FIG. 5A shows multiple alignment of maize (SEQ ID NO: 1), sorghum (SEQ ID NO: 6), rice (SEQ ID NO: 7), soybean (SEQ ID NO: 9), and Arabidopsis (SEQ ID NO: 8) from amino acid (aa) 1 to aa 400. FIG. 5B continues the alignment from aa 401 to aa 467. FIG. 5C shows the phylogenetic relationship of maize brachytic 1 gene with its homologs from other plants species.

FIG. 6A shows multiple alignment of RT-PCR transcripts amplified from TX430 (SEQ ID NOs: 91 and 108) and P898012 (SEQ ID NOs: 109 and 110) sorghum lines and the sorghum SbBR1 (Sb07G021280) sequence (SEQ ID NO: 17) from aa 1 to aa 450. FIG. 6B continues the alignment from amino acid 451 to aa 850. FIG. 6C continues the alignment from aa 851 to aa 1250. FIG. 6D continues the alignment from aa 1251 to aa 1481.

FIG. 7A shows multiple sequence alignments of predicted peptides of differentially spliced Transcripts of TX430 and P898012 compared with wild-type peptide from aa 1 to aa 450. FIG. 7B continues the alignment from aa 451 to aa 507. Sb07021280-dw is SEQ ID NO: 6, P898012-dw is SEQ ID NO: 88, P898012-dw-alt is SEQ ID NO: 90, TX430-dw is SEQ ID NO: 92, and TX430-dw-alt is SEQ ID NO: 107.

FIG. 8 shows target sites for genome editing in the Br1 genomic locus. CR-CRISPR sites and corresponding sequences are in Table 1.

FIG. 9 shows CRISPR (CR) target sites in ZmBr1 genomic DNA.

FIG. 10 shows Br1 target sites selected for deletion (SDN1) analysis. CRs targeting both the beginning of exon 2 and close to the end of exon 3 were selected—as these were conserved across genotypes. CR Selection: Pair 1: CR3 & CR5-Deletion of 1899 bp (EXX-INBRED) and 1910 bp (GXX-INBRED); Pair 2: CR4 & CR6-Deletion of 1894 bp (EXX-INBRED) and 1905 bp (GXX-INBRED). No unintended ORFs >450 bp is created with perfect repair. Small portion of Exon 2 remains: Pair 1-46 bp, Pair 2-104 bp. C-terminus of Exon 3 remains: Pair 1-86 bp, Pair 2-33 bp (includes WT stop).

FIG. 11 shows Zm-BR1 exons 2 and 3 deletion schematic for SDN1 approach. Recessive mutations target both in SS (EXX-inbred; stiff stalk) and NSS (GXX-inbred non-stiff stalk) elite lines. Two pairs dual gRNAs are shown: CR3 & CR5, CR4 & CR6 to create a non-functional mutant allele at br1 locus through deletion of exon 3 or deletion of exon 2 as well as exon 3 (SDN1).

FIG. 12 shows a schematic representing Br1-Mutag insertion. To create a weak mutant allele which would behave as semi-dwarf but still recessive mutant, thus, targeting both SS (EXX-inbred; stiff stalk) and NSS (GXX-inbred non-stiff stalk) genotypes, insertion of Br1-mutag is performed. br1-mutag allele: Addition of 143 bp of MuTIR in intron1 would interfere in splicing. One gRNA (CR3 or CR4) will be used to have a single cut in exon2 and BR1-Mutag transcript; (ZM-BR1-ALT1) sequence is added by homology directed repair.

FIG. 13 shows a schematic representing genomic edit structure and insertion of Mu-Tag fragment by Cas9 SDN2: A construct showing 143 bp of Mu-TIR (middle part) (SEQ ID NO: 111) flanked by left and right homologous arms of 500 bp size (ZM-BR1-ALT1) for recombination was inserted at CR3 site. Mu-TIRa (SEQ ID NO:30) and Mu-TIRb (SEQ ID NO: 31) primer sequences are shown.

FIG. 14 shows gene structure and target locations in SbDw3 (A) and SbDw5 (B). Various gRNA target sites are also shown (sequences in Table 4).

FIG. 15 shows RT-PCR expression analysis using GSPs from the flanking 5′-UTR and 3′-UTR sequences of br1 gene.

The sequence descriptions summarize the Sequence Listing attached hereto, which is hereby incorporated by reference. The Sequence Listing contains one letter codes for nucleotide sequence characters and the single and three letter codes for amino acids as defined in the IUPAC-IUB standards described in Nucleic Acids Research 13:3021-3030 (1985) and in the Biochemical Journal 219 (2):345-373 (1984).

TABLE 1
Sequence Listing Description

| SEQ ID NOS | Description |
|---|---|
| SEQ ID NO: 1 | dpzm01g068810.1.1 Peptide Transcription regulator HTH (ZmBr1) |
| SEQ ID NO: 2 | dpzm01g068810.1.2 Peptide Transcription regulator HTH |
| SEQ ID NO: 3 | dpzm01g068810.1.3 Peptide Transcription regulator HTH |
| SEQ ID NO: 4 | dpzm01g068810.1.4 Peptide Transcription regulator HTH |
| SEQ ID NO: 5 | Predicted amino acid sequence of br1-Mutag allele |
| SEQ ID NO: 6 | Sb07g021280.1 peptide |
| SEQ ID NO: 7 | Os08g33800.1(Q6YW39) peptide |
| SEQ ID NO: 8 | AT1G69560.1 (ATMYB105) peptide |
| SEQ ID NO: 9 | Glyma01g05980.1 peptide |
| SEQ ID NO: 10 | dpzm01g068810.1.1 gDNA Myb-type (ZmBr1) |
| SEQ ID NO: 11 | dpzm01g068810.1.1 CDS Myb-type (ZmBr1) |
| SEQ ID NO: 12 | dpzm01g068810.1.2 CDS Transcription regulator HTH |
| SEQ ID NO: 13 | dpzm01g068810.1.3 CDS Transcription regulator HTH |
| SEQ ID NO: 14 | dpzm01g068810.1.4 CDS Transcription regulator HTH |
| SEQ ID NO: 15 | Sequence of a novel RTE present in br1-CooP mutant allele |
| SEQ ID NO: 16 | Sequence of RT-PCR amplified br1-Mutag transcript |
| SEQ ID NO: 17 | Sb07g021280.1 CDS |
| SEQ ID NO: 18 | Os08g33800.1(Q6YW39) CDS |
| SEQ ID NO: 19 | AT1G69560.1 (ATMYB105)CDS |
| SEQ ID NO: 20 | Glyma01g05980.1 CDS |
| SEQ ID NO: 21 | Sequence of SDN3 variant amplified by GSPs (GSP7 + GSP8) |
| SEQ ID NO: 22 | GSP1 |
| SEQ ID NO: 23 | GSP2 |
| SEQ ID NO: 24 | GSP3 |
| SEQ ID NO: 25 | GSP4 |
| SEQ ID NO: 26 | GSP5 |
| SEQ ID NO: 27 | GSP6 |
| SEQ ID NO: 28 | GSP7 |
| SEQ ID NO: 29 | GSP8 |
| SEQ ID NO: 30 | Mu-TIRa |
| SEQ ID NO: 31 | Mu-TIRb |
| SEQ ID NO: 32 | ZM-BR1-CR1-sense |
| SEQ ID NO: 33 | ZM-BR1-CR2-complement |
| SEQ ID NO: 34 | ZM-BR1-CR3-complement |
| SEQ ID NO: 35 | ZM-BR1-CR4-sense |
| SEQ ID NO: 36 | ZM-BR1-CR5-sense |
| SEQ ID NO: 37 | ZM-BR1-CR6-complement |
| SEQ ID NO: 38 | ZM-BR1-CR7 |
| SEQ ID NO: 39 | ZM-BR1-CR8 |
| SEQ ID NO: 40 | ZM-BR1-CR9 |
| SEQ ID NO: 41 | ZM-BR2-CR1 |
| SEQ ID NO: 42 | ZM-BR2-CR3 |
| SEQ ID NO: 43 | Zm-BR2-PRT-full |
| SEQ ID NO: 44 | Zm-Br2-genomic-full |
| SEQ ID NO: 45 | Zm-Br2-cDNA |
| SEQ ID NO: 46 | ZM-BR2-CR4 |
| SEQ ID NO: 47 | ZM-BR2-CR5 |
| SEQ ID NO: 48 | ZM-BR2-CR6 |
| SEQ ID NO: 49 | ZM-BR2-CR7 |
| SEQ ID NO: 50 | ZM-BR2-CR8 |
| SEQ ID NO: 51 | ZM-BR2-CR9 |
| SEQ ID NO: 52 | ZM-BR2-CR10 |
| SEQ ID NO: 53 | ZM-BR2-CR11 |
| SEQ ID NO: 54 | ZM-BR2-CR12 |
| SEQ ID NO: 55 | ZM-BR2-CR13 |
| SEQ ID NO: 56 | ZM-BR2-CR14 |
| SEQ ID NO: 57 | ZM-BR2-CR15 |
| SEQ ID NO: 58 | ZM-BR2-CR16 |
| SEQ ID NO: 59 | ZM-BR2-CR17 |
| SEQ ID NO: 60 | ZM-BR2-CR18 |
| SEQ ID NO: 61 | ZM-BR2-CR12 + CR13 |
| SEQ ID NO: 62 | ZM-D8-CR2 |
| SEQ ID NO: 63 | ZM-D8-CR3 |
| SEQ ID NO: 64 | ZM-D8-CR4 |
| SEQ ID NO: 65 | ZM-D8-CR5 |
| SEQ ID NO: 66 | ZM-D8-CR6 |
| SEQ ID NO: 67 | ZM-D8-CR7 |
| SEQ ID NO: 68 | ZM-D8-CR8 |
| SEQ ID NO: 69 | ZM-D8-CR9 |
| SEQ ID NO: 70 | ZM-BR1_CDS_B73 |
| SEQ ID NO: 71 | ZM-BR1_PRO_B73 |
| SEQ ID NO: 72 | ZM-BR2_PRO_B73 |
| SEQ ID NO: 73 | ZM-D8_CDS_B73 |
| SEQ ID NO: 74 | ZM-D8_GENE_B73 |
| SEQ ID NO: 75 | ZM-D8_PRO_B73 |
| SEQ ID NO: 76 | ZM-D8-Peptide-B73 |
| SEQ ID NO: 77 | Br1 18-bp insertion haplotype |
| SEQ ID NO: 78 | DW3-TS1 gRNA |
| SEQ ID NO: 79 | DW3-TS2 gRNA |
| SEQ ID NO: 80 | DW3-TS3 gRNA |
| SEQ ID NO: 81 | DW3-TS4 gRNA |
| SEQ ID NO: 82 | DW5-TS1 gRNA |
| SEQ ID NO: 83 | DW5-TS2 gRNA |
| SEQ ID NO: 84 | DW5-TS3 gRNA |
| SEQ ID NO: 85 | DW5-TS4 gRNA |
| SEQ ID NO: 86 | SbDw5 genomic sequence |
| SEQ ID NO: 87 | SbDw5-mRNA sequence |
| SEQ ID NO: 88 | SbDW5 peptide sequence |
| SEQ ID NO: 89 | Differential spliced transcript sorghum dw5 (dw5-ALT) |
| SEQ ID NO: 90 | Peptide of differential sliced sorghum dw5 (DW5-ALT) |
| SEQ ID NO: 91 | SbDw5-mRNA of TX430 |
| SEQ ID NO: 92 | SbDW5 peptide of TX430 |
| SEQ ID NO: 93 | SbDw3 genomic sequence |
| SEQ ID NO: 94 | SbDw3-mRNA sequence |
| SEQ ID NO: 95 | SbDW3 peptide sequence |
| SEQ ID NO: 96 | Dw3-TS1 Forward Primer |
| SEQ ID NO: 97 | Dw3-TS1 Reverse Primer |
| SEQ ID NO: 98 | Dw3-TS3 Forward Primer |
| SEQ ID NO: 99 | Dw3-TS3 Reverse Primer |
| SEQ ID NO: 100 | Os09g01960.1 rice amino acid ortholog of ZmBr1 |
| SEQ ID NO: 101 | Os01g16810.1 rice amino acid ortholog of ZmBr1 |
| SEQ ID NO: 102 | Os11g10130 rice amino acid ortholog of ZmBr1 |
| SEQ ID NO: 103 | Os03g51110.1 rice amino acid ortholog of ZmBr1 |
| SEQ ID NO: 104 | Sb03g010960.1 sorghum amino acid ortholog of ZmBr1 |
| SEQ ID NO: 105 | Sb03g010970.1 sorghum amino acid ortholog of ZmBr1 |
| SEQ ID NO: 106 | Sb007G137200 sorghum amino acid ortholog of ZmBrl |
| SEQ ID NO: 107 | Tx430-dw5-alt peptide |
| SEQ ID NO: 108 | Tx430-dw5-alt cds |

### DETAILED DESCRIPTION

The disclosure of all patents, patent applications, and publications cited herein are incorporated by reference in their entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

### Gibberellin Biosynthesis and Deactivation

Gibberellins have been identified as determinants of plant height. Mutants such as sd1 in rice, rht-1 in wheat or barley sdw1 map to genes involved in gibberellin synthesis or signaling. Gibberellins (GA) are plant hormones involved multiple processes of plant growth and development: germination, stem elongation, leaf expansion, flowering. Among the large number of GA species that have been identified, a few forms are thought to be biologically active (GA1, GA3, GA4, GA7). Some of the enzymes involved in GA biosynthesis are GA20-oxidases (GA200x), GA3-oxidases (GA3ox) and GA2-oxidases (GA2ox). Disruption of these enzymes affects plant stature. GA20ox and GA3ox catalyze oxidations which convert inactive GAs into active GAs (GA20, GA1, GA4) and thus enhance GA responses. GA2ox deactivates GAs by converting GA4 and GA1 into inactive forms. Therefore, methods and compositions are provided that modulate the expression levels, activity levels and a combination thereof of GA these GA biosynthetic pathway that impact plant stature. More specifically, genome edited variants are provided that affect GA biosynthesis, GA signaling and/or a combination thereof.

### DELLA Proteins and Regulators of GA Responses

DELLA proteins are a subfamily of the GRAS superfamily of proteins and play an important role in the negative regulation of GA signaling. In presence of GAs, DELLAs associate with GID1 (Gibberellin insensitive dwarf1), and are then ubiquitinated and degraded through 26S proteasome pathway and subsequent de-repression of downstream effectors of the GA pathway. DELLAs operate in the nucleus and function as transcriptional regulators. They are considered a central switch of GA action and it has been suggested that GA3ox and GA20ox might be some of the direct targets of DELLA proteins.

In addition to DELLAs, feedback regulators of GA biosynthesis such as for example, RSG (Repression of Shoot Growth), a bZIP transcription factor, and its interactors 14-3-3, SCL3 (Scarecrow-like3), another member of the GRAS family, have been identified as GA regulators. Therefore, methods and compositions are provided that modulate the expression levels, activity levels and a combination thereof of GA regulators such as DELLA are that impact plant stature. More specifically, genome edited variants are provided that affect GA regulation, GA signaling and/or a combination thereof.

### Brassinosteroids

Brassinosteroids, and its most active form brassinolide, are a group of steroid hormones that have been identified in many plant species. In addition to gibberellins, brassinosteroid-deficient mutants have also been a significant source of dwarfism in crops such as barley. The uzu-type barley, which is insensitive to brassinosteroid treatment, has lodging resistance and upright leaf angle. Uzu1 was identified as an ortholog of Arabidopsis BRI1 and rice D61, encoding the brassinosteroid receptor. Brassinosteroid mutants typically show shortened upper internodes and shorter grain, along with upright leaves. They also tend to exhibit delayed flowering time and leaf senescence. In addition to the biosynthetic pathway, perception and signal transduction of the Brassinosteroid pathway are amenable to manipulation using the methods and compositions provided herein for modulating stature. BRI1, BRL1, BRL3 receptors are also suitable candidates for genome editing to improve stature, for example, by reducing plant height. Weaker alleles of the Brassinosteroid biosynthetic enzymes or targeting genes downstream of the major steps of the biosynthesis pathway may be helpful ways to address reducing plant height by modulating Brassinosteroid pathway, wherein the plant height reduction is not severe and semi-dwarf phenotype is obtained.

### Shade Avoidance and Photomorphogenesis

Methods and compositions to develop plants that respond to shade and plant-to-plant competition by modification of one or more genetic loci to modulate various sets of processes including cell elongation, flowering time, resource partitioning and apical dominance. Modifying plant growth at the canopy level include, for example, targeting genomic edits in genes involved in photomorphogenesis. For example, Phytochrome Interacting Factors (PIFs) involved in the regulation of auxin production are suitable target candidates for improving plant responses to shade.

### Yield and Plant Height Manipulation

Altering plant height may affect yield (or one of its components such as kernel size, kernel weight), as these traits are correlated. Therefore, methods and compositions are provided herein to manipulate plant height without a substantial reduction in grain yield through selective editing genomic loci, e.g., by creating weaker alleles of genomic regions involved dwarfism. For example, one or more variants created through genome editing techniques help separate genetically linked regions of the genome that control yield components and height, i.e., without incurring a yield penalty. In addition, smaller yield losses may be offset at the population level by reduced lodging and increased planting densities. Methods and compositions are provided that either decrease yield penalty on a per plant basis or minimizing overall yield loss by decreased lodging and tolerance to higher planting densities.

### Flowering Time

Methods and compositions are provided herein that impact plant height without substantially altering desirable flowering time window, e.g., semi-dwarf inbreds with a deviation of flowering time of about plus or minus 10-20 CRM. By targeted modification of one or more genomic loci involved in height reduction and uncoupling such height reduction from flowering time impact, overall yield is increased.

### Plant Growth and Development

Gene editing targets and methods to generate weaker variants of genomic regions that control plant height help develop agronomically relevant mutant plants. For example, weaker alleles of previously known are generated through genome editing. In other cases, weaker of alleles of previously unknown targets are also generated that reduce plant height with minimal pleiotropic effects.

A “Br1 mutant plant” or a “Br1 plant” or a “Br1 modified plant” or “br1” generally refers to a modified plant or mutant plant that has one or more nucleotide changes in a genomic region that encodes a polypeptide that is at least 80% identical to one of SEQ ID NOS: 1-9 or an allelic variant thereof, wherein the plant shows altered stature including for example reduced plant height and/or ear height.

An “isolated polynucleotide” generally refers to a polymer of ribonucleotides (RNA) or deoxyribonucleotides (DNA) that is single- or double-stranded, optionally containing synthetic, non-natural or altered nucleotide bases. An isolated polynucleotide in the form of DNA may be comprised of one or more segments of cDNA, genomic DNA or synthetic DNA.

The terms “polynucleotide”, “polynucleotide sequence”, “nucleic acid sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

A regulatory element generally refers to a transcriptional regulatory element involved in regulating the transcription of a nucleic acid molecule such as a gene or a target gene. The regulatory element is a nucleic acid and may include a promoter, an enhancer, an intron, a 5′-untranslated region (5′-UTR, also known as a leader sequence), or a 3′-UTR or a combination thereof. A regulatory element may act in “cis” or “trans”, and generally it acts in “cis”, i.e. it activates expression of genes located on the same nucleic acid molecule, e.g. a chromosome, where the regulatory element is located. The nucleic acid molecule regulated by a regulatory element does not necessarily have to encode a functional peptide or polypeptide, e.g., the regulatory element can modulate the expression of a short interfering RNA or an anti-sense RNA.

An enhancer element is any nucleic acid molecule that increases transcription of a nucleic acid molecule when functionally linked to a promoter regardless of its relative position. An enhancer may be an innate element of the promoter or a heterologous element inserted to enhance the level or tissue-specificity of a promoter.

A repressor (also sometimes called herein silencer) is defined as any nucleic acid molecule which inhibits the transcription when functionally linked to a promoter regardless of relative position.

“Promoter” generally refers to a nucleic acid fragment capable of controlling transcription of another nucleic acid fragment. A promoter generally includes a core promoter (also known as minimal promoter) sequence that includes a minimal regulatory region to initiate transcription, that is a transcription start site. Generally, a core promoter includes a TATA box and a GC rich region associated with a CAAT box or a CCAAT box. These elements act to bind RNA polymerase II to the promoter and assist the polymerase in locating the RNA initiation site. Some promoters may not have a TATA box or CAAT box or a CCAAT box, but instead may contain an initiator element for the transcription initiation site. A core promoter is a minimal sequence required to direct transcription initiation and generally may not include enhancers or other UTRs. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Core promoters are often modified to produce artificial, chimeric, or hybrid promoters, and can further be used in combination with other regulatory elements, such as cis-elements, 5′UTRs, enhancers, or introns, that are either heterologous to an active core promoter or combined with its own partial or complete regulatory elements.

The term “cis-element” generally refers to transcriptional regulatory element that affects or modulates expression of an operably linked transcribable polynucleotide, where the transcribable polynucleotide is present in the same DNA sequence. A cis-element may function to bind transcription factors, which are trans-acting polypeptides that regulate transcription.

“Promoter functional in a plant” is a promoter capable of initiating transcription in plant cells whether or not its origin is from a plant cell.

“Tissue-specific promoter” and “tissue-preferred promoter” are used interchangeably to refer to a promoter that is expressed predominantly but not necessarily exclusively in one tissue or organ, but that may also be expressed in one specific cell.

“Developmentally regulated promoter” generally refers to a promoter whose activity is determined by developmental events.

“Constitutive promoter” generally refers to promoters active in all or most tissues or cell types of a plant at all or most developing stages. As with other promoters classified as “constitutive” (e.g. ubiquitin), some variation in absolute levels of expression can exist among different tissues or stages. The term “constitutive promoter” or “tissue-independent” are used interchangeably herein.

A “heterologous nucleotide sequence” generally refers to a sequence that is not naturally occurring with the sequence of the disclosure. While this nucleotide sequence is heterologous to the sequence, it may be homologous, or native, or heterologous, or foreign, to the plant host. However, it is recognized that the instant sequences may be used with their native coding sequences to increase or decrease expression resulting in a change in phenotype in the transformed seed. The terms “heterologous nucleotide sequence”, “heterologous sequence”, “heterologous nucleic acid fragment”, and “heterologous nucleic acid sequence” are used interchangeably herein.

A “functional fragment” refers to a portion or subsequence of the sequence described in the present disclosure in which, the ability to modulate gene expression is retained. Fragments can be obtained via methods such as site-directed mutagenesis and synthetic construction. As with the provided promoter sequences described herein, the functional fragments operate to promote the expression of an operably linked heterologous nucleotide sequence, forming a recombinant DNA construct (also, a chimeric gene). For example, the fragment can be used in the design of recombinant DNA constructs to produce the desired phenotype in a transformed plant. Recombinant DNA constructs can be designed for use in co-suppression or antisense by linking a promoter fragment in the appropriate orientation relative to a heterologous nucleotide sequence.

A nucleic acid fragment that is functionally equivalent to the Target sequences of the present disclosure is any nucleic acid fragment that is capable of modulating the expression of a coding sequence or functional RNA in a similar manner to the Target sequences of the present disclosure.

The polynucleotide sequence of the targets of the present disclosure (e.g., SEQ ID NOS: 1-84), may be modified or altered to enhance their modulation characteristics. As one of ordinary skill in the art will appreciate, modification or alteration can also be made without substantially affecting the gene expression function. The methods are well known to those of skill in the art. Sequences can be modified, for example by insertion, deletion, or replacement of template sequences through any modification approach.

A “variant promoter” as used herein, is the sequence of the promoter or the sequence of a functional fragment of a promoter containing changes in which one or more nucleotides of the original sequence is deleted, added, and/or substituted, while substantially maintaining promoter function. One or more base pairs can be inserted, deleted, or substituted internally to a promoter. In the case of a promoter fragment, variant promoters can include changes affecting the transcription of a minimal promoter to which it is operably linked. Variant promoters can be produced, for example, by standard DNA mutagenesis techniques or by chemically synthesizing the variant promoter or a portion thereof.

Modifying stature of plants by one or more methods and compositions disclosed here are characterized by one or more of the following traits: a shorter stature or semi-dwarf plant height, reduced internode length, increased stalk/stem diameter, improved lodging resistance, reduced green snap, deeper roots, increased leaf area, earlier canopy closure, altered foliar water content and/or higher stomatal conductance under water/nutrient limiting conditions, improved yield-related traits including a larger female reproductive part of a plant e.g., corn car or sorghum tiller, panicle, an increase in ear weight, harvest index, yield, seed or kernel number/panicle number, and/or seed or kernel weight, relative to a wild type or control plant. Increased stress tolerance e.g., drought tolerance, nitrogen utilization, and/or tolerance to higher planting density are contemplated.

In some aspects of the present disclosure, the fragments of polynucleotide sequences disclosed herein can comprise at least about 20 contiguous nucleotides, or at least about 50 contiguous nucleotides, or at least about 75 contiguous nucleotides, or at least about 100 contiguous nucleotides, or at least about 150 contiguous nucleotides, or at least about 200 contiguous nucleotides of nucleic acid sequences or polypeptides encoded designated by the SEQ ID NOS: listed in Table 1. In another aspect of the present disclosure, the fragments can comprise at least about 250 contiguous nucleotides, or at least about 300 contiguous nucleotides, or at least about 350 contiguous nucleotides, or at least about 400 contiguous nucleotides, or at least about 450 contiguous nucleotides, or at least about 500 contiguous nucleotides, or at least about 550 contiguous nucleotides, or at least about 600 contiguous nucleotides, or at least about 650 contiguous nucleotides, or at least about 700 contiguous nucleotides, or at least about 750 contiguous nucleotides, or at least about 800 contiguous nucleotides, or at least about 850 contiguous nucleotides, or at least about 900 contiguous nucleotides, or at least about 950 contiguous nucleotides, or at least about 1000 contiguous nucleotides, or at least about 1050 contiguous nucleotides and further may include a sequence from Table 1 listings.

The terms “full complement” and “full-length complement” are used interchangeably herein, and refer to a complement of a given nucleotide sequence, wherein the complement and the nucleotide sequence consist of the same number of nucleotides and are 100% complementary.

The terms “substantially similar” and “corresponding substantially” as used herein refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences.

The transitional phrase “consisting essentially of” generally refers to a composition, method that includes materials, steps, features, components, or elements, in addition to those literally disclosed, provided that these additional materials, steps, features, components, or elements do not materially affect the basic and novel characteristic(s) of the claimed subject matter, e.g., one or more of the claimed sequences.

The isolated promoter sequence comprised in the recombinant DNA construct of the present disclosure can be modified to provide a range of constitutive expression levels of the heterologous nucleotide sequence. Thus, less than the entire promoter regions may be utilized and the ability to drive expression of the coding sequence retained. However, it is recognized that expression levels of the mRNA may be decreased with deletions of portions of the promoter sequences. Likewise, the tissue-independent, constitutive nature of expression may be changed.

Modifications of the isolated promoter sequences of the present disclosure can provide for a range of constitutive expression of the heterologous nucleotide sequence. Thus, they may be modified to be weak constitutive promoters or strong constitutive promoters. Generally, by “weak promoter” is intended a promoter that drives expression of a coding sequence at a low level. By “low level” is intended levels about 1/10,000 transcripts to about 1/100,000 transcripts to about 1/500,000 transcripts. Conversely, a strong promoter drives expression of a coding sequence at high level, or at about 1/10 transcripts to about 1/100 transcripts to about 1/1,000 transcripts. Similarly, a “moderate constitutive” promoter is somewhat weaker than a strong constitutive promoter like the maize ubiquitin promoter.

Planting density in a field, e.g., may range from about at least 36,000 plants per acre, at least 40,000 plants per acre, at least 42,000 plants per acre, at least 44,000 plants per acre, at least 45,000 plants per acre, at least 46,000 plants per acre, at least 48,000 plants per acre, 50,000 plants per acre, at least 52,000 plants per acre, at least 54,000 per acre, or at least 56,000 plants per acre. In an embodiment, corn plants may be planted at a higher density, such as in a range from about 36,000 plants per acre to about 60,000 plants per acre, or about 40,000 plants per acre to about 58,000 plants per acre, or about 42,000 plants per acre to about 58,000 plants per acre, or about 40,000 plants per acre to about 45,000 plants per acre, or about 45,000 plants per acre to about 50,000 plants per acre, or about 50,000 plants per acre to about 58,000 plants per acre, or about 52,000 plants per acre to about 56,000 plants per acre, or about 38,000 plants per acre, about 42,000 plant per acre, about 46,000 plant per acre, or about 48,000 plants per acre, about 50,000 plants per acre, or about 52,000 plants per acre, or about 54,000 plant per acre, as opposed to a standard planting density range, such as about 18,000 plants per acre to about 38,000 plants per acre.

In addition to modulating gene expression, the expression modulating elements disclosed herein are also useful as probes or primers in nucleic acid hybridization experiments. The nucleic acid probes and primers hybridize under stringent conditions to a target DNA sequence. A “probe” is generally referred to an isolated/synthesized nucleic acid to which, is attached a conventional detectable label or reporter molecule, such as for example, a radioactive isotope, ligand, chemiluminescent agent, bioluminescent molecule, fluorescent label or dye, or enzyme. Such detectable labels may be covalently linked or otherwise physically associated with the probe. “Primers” generally referred to isolated/synthesized nucleic acids that hybridize to a complementary target DNA strand which is then extended along the target DNA strand by a polymerase, e.g., a DNA polymerase. Primer pairs often used for amplification of a target nucleic acid sequence, e.g., by the polymerase chain reaction (PCR) or other conventional nucleic-acid amplification methods. Primers are also used for a variety of sequencing reactions, sequence captures, and other sequence-based amplification methodologies. Primers are generally about 15, 20, 25 nucleotides or more, and probes can also be longer about 30, 40, 50 and up to a few hundred base pairs. Such probes and primers are used in hybridization reactions to target DNA or RNA sequences under high stringency hybridization conditions or under lower stringency conditions, depending on the need.

Moreover, the skilled artisan recognizes that substantially similar nucleic acid sequences encompassed by this disclosure are also defined by their ability to hybridize, under moderately stringent conditions (for example, 0.5×SSC, 0.1% SDS, 60° C.) with the sequences exemplified herein, or to any portion of the nucleotide sequences reported herein and which are functionally equivalent to the promoter of the disclosure. Estimates of such homology are provided by either DNA-DNA or DNA-RNA hybridization under conditions of stringency as is well understood by those skilled in the art (Hames and Higgins, Eds.; In Nucleic Acid Hybridisation; IRL Press: Oxford, U. K., 1985). Stringency conditions can be adjusted to screen for moderately similar fragments, such as homologous sequences from distantly related organisms, to highly similar fragments, such as genes that duplicate functional enzymes from closely related organisms. Post-hybridization washes partially determine stringency conditions. One set of conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. Another set of stringent conditions uses higher temperatures in which the washes are identical to those above except for the temperature of the final two 30 min washes in 0.2×SSC, 0.5% SDS was increased to 60° C. Another set of highly stringent conditions uses two final washes in 0.1×SSC, 0.1% SDS at 65° C.

Preferred substantially similar nucleic acid sequences encompassed by this disclosure are those sequences that are 80% identical to the nucleic acid fragments reported herein or which are 80% identical to any portion of the nucleotide sequences reported herein. More preferred are nucleic acid fragments which are 90% identical to the nucleic acid sequences reported herein, or which are 90% identical to any portion of the nucleotide sequences reported herein. Most preferred are nucleic acid fragments which are 95% identical to the nucleic acid sequences reported herein, or which are 95% identical to any portion of the nucleotide sequences reported herein. It is well understood by one skilled in the art that many levels of sequence identity are useful in identifying related polynucleotide sequences. Useful examples of percent identities are those listed above, or also preferred is any integer percentage from 71% to 100%, such as 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

In one embodiment, the isolated sequences of the present disclosure comprises a nucleotide sequence having at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% sequence identity, based on the Clustal V method of alignment with pairwise alignment default parameters (KTUPLE=2, GAP PENALTY=5, WINDOW=4 and DIAGONALS SAVED=4), when compared to the nucleotide sequence of SEQ ID NOS: 1-52. It is known to one of skilled in the art that a 5′ UTR region can be altered (deletion or substitutions of bases) or replaced by an alternative 5′UTR while maintaining promoter activity.

A “substantially similar sequence” generally refers to variants of the disclosed sequences such as those that result from site-directed mutagenesis, as well as synthetically derived sequences. A substantially similar promoter sequence of the present disclosure also generally refers to those fragments of a particular promoter nucleotide sequence disclosed herein that operate to promote the constitutive expression of an operably linked heterologous nucleic acid fragment. These promoter fragments comprise at least about 20 contiguous nucleotides, at least about 50 contiguous nucleotides, at least about 75 contiguous nucleotides, preferably at least about 100 contiguous nucleotides of the particular promoter nucleotide sequence disclosed herein or a sequence that is at least 95 to about 99% identical to such contiguous sequences. The nucleotides of such fragments will usually include the TATA recognition sequence (or CAAT box or a CCAAT) of the particular promoter sequence. Such fragments may be obtained by use of restriction enzymes to cleave the naturally occurring promoter nucleotide sequences disclosed herein; by synthesizing a nucleotide sequence from the naturally occurring promoter DNA sequence; or may be obtained through the use of PCR technology. Variants of these promoter fragments, such as those resulting from site-directed mutagenesis, are encompassed by the compositions of the present disclosure.

“Codon degeneracy” generally refers to divergence in the genetic code permitting variation of the nucleotide sequence without affecting the amino acid sequence of an encoded polypeptide. Accordingly, the instant disclosure relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for improved expression in a host cell, it is desirable to design the nucleic acid fragment such that its frequency of codon usage approaches the frequency of preferred codon usage of the host cell.

Sequence alignments and percent identity calculations may be determined using a variety of comparison methods designed to detect similar or identical sequences including, but not limited to, the Megalign® program of the LASERGENE® bioinformatics computing suite (DNASTAR® Inc., Madison, WI). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

In one embodiment the % sequence identity is determined over the entire length of the molecule (nucleotide or amino acid). A “substantial portion” of an amino acid or nucleotide sequence comprises enough of the amino acid sequence of a polypeptide or the nucleotide sequence of a gene to afford putative identification of that polypeptide or gene, either by manual evaluation of the sequence by one skilled in the art, or by computer-automated sequence comparison and identification using algorithms such as BLAST (Altschul, S. F. et al., J. Mol. Biol. 215:403-410 (1993)) and Gapped Blast (Altschul, S. F. et al., Nucleic Acids Res. 25:3389-3402 (1997)). BLASTN generally refers to a BLAST program that compares a nucleotide query sequence against a nucleotide sequence database.

“Gene” includes a nucleic acid fragment that expresses a functional molecule such as, but not limited to, a specific protein, including regulatory sequences preceding (5′ non-coding sequences) and following (3′ non-coding sequences) the coding sequence. “Native gene” generally refers to a gene as found in nature with its own regulatory sequences.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas endonuclease system as disclosed herein. A mutated plant is a plant comprising a mutated gene.

“Chimeric gene” or “recombinant expression construct”, which are used interchangeably, includes any gene that is not a native gene, comprising regulatory and coding sequences that are not found together in nature. Accordingly, a chimeric gene may comprise regulatory sequences and coding sequences that are derived from different sources.

“Coding sequence” generally refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

An “intron” is an intervening sequence in a gene that is transcribed into RNA but is then excised in the process of generating the mature mRNA. The term is also used for the excised RNA sequences. An “exon” is a portion of the sequence of a gene that is transcribed and is found in the mature messenger RNA derived from the gene, but is not necessarily a part of the sequence that encodes the final gene product.

The 5′ untranslated region (5′UTR) (also known as a translational leader sequence or leader RNA) is the region of an mRNA that is directly upstream from the initiation codon. This region is involved in the regulation of translation of a transcript by differing mechanisms in viruses, prokaryotes and eukaryotes.

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“RNA transcript” generally refers to a product resulting from RNA polymerase-catalyzed transcription of a DNA sequence. When an RNA transcript is a perfect complimentary copy of a DNA sequence, it is referred to as a primary transcript or it may be a RNA sequence derived from posttranscriptional processing of a primary transcript and is referred to as a mature RNA. “Messenger RNA” (“mRNA”) generally refers to RNA that is without introns and that can be translated into protein by the cell. “cDNA” generally refers to a DNA that is complementary to and synthesized from an mRNA template using the enzyme reverse transcriptase. The cDNA can be single-stranded or converted into the double-stranded by using the Klenow fragment of DNA polymerase I. “Sense” RNA generally refers to RNA transcript that includes mRNA and so can be translated into protein within a cell or in vitro. “Antisense RNA” generally refers to a RNA transcript that is complementary to all or part of a target primary transcript or mRNA and that blocks expression or transcripts accumulation of a target gene. The complementarity of an antisense RNA may be with any part of the specific gene transcript, i.e. at the 5′ non-coding sequence, 3′ non-coding sequence, introns, or the coding sequence. “Functional RNA” generally refers to antisense RNA, ribozyme RNA, or other RNA that may not be translated but yet has an effect on cellular processes.

The term “operably linked” or “functionally linked” generally refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The terms “initiate transcription”, “initiate expression”, “drive transcription”, and “drive expression” are used interchangeably herein and all refer to the primary function of a promoter. As detailed throughout this disclosure, a promoter is a non-coding genomic DNA sequence, usually upstream (5′) to the relevant coding sequence, and its primary function is to act as a binding site for RNA polymerase and initiate transcription by the RNA polymerase. Additionally, there is “expression” of RNA, including functional RNA, or the expression of polypeptide for operably linked encoding nucleotide sequences, as the transcribed RNA ultimately is translated into the corresponding polypeptide.

The term “expression”, as used herein, generally refers to the production of a functional end-product e.g., an mRNA or a protein (precursor or mature).

The term “expression cassette” as used herein, generally refers to a discrete nucleic acid fragment into which a nucleic acid sequence or fragment can be cloned or synthesized through molecular biology techniques.

Expression or overexpression of a gene involves transcription of the gene and translation of the mRNA into a precursor or mature protein. “Antisense inhibition” generally refers to the production of antisense RNA transcripts capable of suppressing the expression of the target protein. “Overexpression” generally refers to the production of a gene product in transgenic organisms that exceeds levels of production in normal or non-transformed organisms. “Co-suppression” generally refers to the production of sense RNA transcripts capable of suppressing the expression or transcript accumulation of identical or substantially similar foreign or endogenous genes (U.S. Pat. No. 5,231,020). The mechanism of co-suppression may be at the DNA level (such as DNA methylation), at the transcriptional level, or at post-transcriptional level.

As stated herein, “suppression” includes a reduction of the level of enzyme activity or protein functionality (e.g., a phenotype associated with a protein) detectable in a transgenic plant when compared to the level of enzyme activity or protein functionality detectable in a non-transgenic or wild type plant with the native enzyme or protein. The level of enzyme activity in a plant with the native enzyme is referred to herein as “wild type” activity. The level of protein functionality in a plant with the native protein is referred to herein as “wild type” functionality. The term “suppression” includes lower, reduce, decline, decrease, inhibit, eliminate and prevent. This reduction may be due to a decrease in translation of the native mRNA into an active enzyme or functional protein. It may also be due to the transcription of the native DNA into decreased amounts of mRNA and/or to rapid degradation of the native mRNA. The term “native enzyme” generally refers to an enzyme that is produced naturally in a non-transgenic or wild type cell. The terms “non-transgenic” and “wild type” are used interchangeably herein.

“Altering expression” or “modulating expression” generally refers to the production of gene product(s) in plants in amounts or proportions that differ significantly from the amount of the gene product(s) produced by the corresponding wild-type plants (i.e., expression is increased or decreased).

“Transformation” as used herein generally refers to both stable transformation and transient transformation.

“Stable transformation” generally refers to the introduction of a nucleic acid fragment into a genome of a host organism resulting in genetically stable inheritance. Once stably transformed, the nucleic acid fragment is stably integrated in the genome of the host organism and any subsequent generation. Host organisms containing the transformed nucleic acid fragments are referred to as “transgenic” organisms. “Transient transformation” generally refers to the introduction of a nucleic acid fragment into the nucleus, or DNA-containing organelle, of a host organism resulting in gene expression without genetically stable inheritance.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

“Genome” as it applies to plant cells encompasses not only chromosomal DNA found within the nucleus, but organelle DNA found within subcellular components (e.g., mitochondrial, plastid) of the cell.

“Genetic modification” generally refers to modification of any nucleic acid sequence or genetic element by insertion, deletion, or substitution of one or more nucleotides in an endogenous nucleotide sequence by genome editing or by insertion of a recombinant nucleic acid, e.g., as part of a vector or construct in any region of the plant genomic DNA by routine transformation techniques. Examples of modification of genetic components include, but are not limited to, promoter regions, 5′ untranslated leaders, introns, genes, 3′ untranslated regions, and other regulatory sequences or sequences that affect transcription or translation of one or more nucleic acid sequences.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

The terms “monocot” and “monocotyledonous plant” are used interchangeably herein. A monocot of the current disclosure includes the Gramineae.

The terms “dicot” and “dicotyledonous plant” are used interchangeably herein. A dicot of the current disclosure includes the following families: Brassicaceae, Leguminosae, and Solanaceae.

“Progeny” comprises any subsequent generation of a plant.

The heterologous polynucleotide can be stably integrated within the genome such that the polynucleotide is passed on to successive generations. The heterologous polynucleotide may be integrated into the genome alone or as part of a recombinant DNA construct. The alterations of the genome (chromosomal or extra-chromosomal) by conventional plant breeding methods, by genome editing procedures that do not result in an insertion of a foreign polynucleotide, or by naturally occurring events such as random cross-fertilization, non-recombinant viral infection, non-recombinant bacterial transformation, non-recombinant transposition, or spontaneous mutation are also methods of modifying a host genome.

“Transient expression” generally refers to the temporary expression of often reporter genes such as β-glucuronidase (GUS), fluorescent protein genes ZS-GREEN1, ZS-YELLOW1 N1, AM-CYAN1, DS-RED in selected certain cell types of the host organism in which the transgenic gene is introduced temporally by a transformation method. The transformed materials of the host organism are subsequently discarded after the transient gene expression assay.

Standard recombinant DNA and molecular cloning techniques used herein are well known in the art and are described more fully in Sambrook, J. et al., In Molecular Cloning: A Laboratory Manual; 2^{nd} ed.; Cold Spring Harbor Laboratory Press: Cold Spring Harbor, New York, 1989 (hereinafter “Sambrook et al., 1989”) or Ausubel, F. M., Brent, R., Kingston, R. E., Moore, D. D., Seidman, J. G., Smith, J. A. and Struhl, K., Eds.; In Current Protocols in Molecular Biology; John Wiley and Sons: New York, 1990 (hereinafter “Ausubel et al., 1990”).

“PCR” or “Polymerase Chain Reaction” is a technique for the synthesis of large quantities of specific DNA segments, consisting of a series of repetitive cycles (Perkin Elmer Cetus Instruments, Norwalk, CT). Typically, the double stranded DNA is heat denatured, the two primers complementary to the 3′ boundaries of the target segment are annealed at low temperature and then extended at an intermediate temperature. One set of these three consecutive steps comprises a cycle.

The terms “plasmid”, “vector” and “cassette” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The term “recombinant DNA construct” or “recombinant expression construct” is used interchangeably and generally refers to a discrete polynucleotide into which a nucleic acid sequence or fragment can be moved. Preferably, it is a plasmid vector or a fragment thereof comprising the promoters of the present disclosure. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain lines displaying the desired expression level and pattern. Such screening may be accomplished by PCR and Southern analysis of DNA, RT-PCR and Northern analysis of mRNA expression, Western analysis of protein expression, or phenotypic analysis.

Various changes in phenotype are of interest including, but not limited to, modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

More specific categories, for example, include, but are not limited to, genes encoding important traits for agronomics, insect resistance, disease resistance, herbicide resistance, sterility, grain or seed characteristics, and commercial products. Genes of interest include, generally, those involved in oil, starch, carbohydrate, or nutrient metabolism as well as those affecting seed size, plant development, plant growth regulation, and yield improvement. Plant development and growth regulation also refer to the development and growth regulation of various parts of a plant, such as the flower, seed, root, leaf and shoot.

Other commercially desirable traits are genes and proteins conferring cold, heat, salt, and drought resistance.

In certain embodiments, the present disclosure contemplates the transformation of a recipient cell with more than one advantageous gene. Two or more genes can be supplied in a single transformation event using either distinct gene-encoding vectors, or a single vector incorporating two or more gene coding sequences. Any two or more genes of any description, such as those conferring herbicide, insect, disease (viral, bacterial, fungal, and nematode), or drought resistance, oil quantity and quality, or those increasing yield or nutritional quality may be employed as desired.

Recombinant DNA constructs comprising an isolated nucleic acid fragment comprising of the targets disclosed herein. This disclosure also concerns a recombinant DNA construct comprising a genomic region of interest of the nucleotide sequence set forth in Table 1.

In another aspect, this disclosure concerns a recombinant DNA construct comprising at least one heterologous nucleic acid fragment operably linked to any promoter, or combination of promoter elements, of the present disclosure. Recombinant DNA constructs can be constructed by operably linking the nucleic acid fragment of the disclosure or a fragment that is substantially similar and functionally equivalent to any portion of the nucleotide sequence set forth in Table 1 to a heterologous nucleic acid fragment. Any heterologous nucleic acid fragment can be used to practice the disclosure. The selection will depend upon the desired application or phenotype to be achieved. The various nucleic acid sequences can be manipulated so as to provide for the nucleic acid sequences in the proper orientation. It is believed that various combinations of promoter elements as described herein may be useful in practicing the present disclosure.

In another aspect, this disclosure concerns a recombinant DNA construct comprising at least one gene that provides drought tolerance operably linked to a heterologous sequence or a fragment, or combination of promoter elements, of the present disclosure. In another aspect, this disclosure concerns a recombinant DNA construct comprising at least one gene that provides insect resistance operably linked to a heterologous sequence or a fragment, or combination of promoter elements, of the present disclosure. In another aspect, this disclosure concerns a recombinant DNA construct comprising at least one gene that increases nitrogen use efficiency and/or yield, operably linked to Target sequences or a fragment, or combination of promoter elements, of the present disclosure. In another aspect, this disclosure concerns a recombinant DNA construct comprising at least one gene that provides herbicide resistance operably linked to Target sequences or a fragment, or combination of promoter elements, of the present disclosure.

In another embodiment, this disclosure concerns host cells comprising either the recombinant DNA constructs of the disclosure as described herein or isolated polynucleotides of the disclosure as described herein. Examples of host cells which can be used to practice the disclosure include, but are not limited to, yeast, bacteria, and plants.

Plasmid vectors comprising the instant recombinant DNA construct can be constructed. The choice of plasmid vector is dependent upon the method that will be used to transform host cells. The skilled artisan is well aware of the genetic elements that must be present on the plasmid vector in order to successfully transform, select and propagate host cells containing the chimeric gene.

### Gene Editing

In some embodiments, gene editing may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpf1 endonuclease systems, and the like. In some embodiments, the introduction of a DSB can be combined with the introduction of a polynucleotide modification template.

A polynucleotide modification template can be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

The polynucleotide modification template can be introduced into a cell as a single stranded polynucleotide molecule, a double stranded polynucleotide molecule, or as part of a circular DNA (vector DNA). The polynucleotide modification template can also be tethered to the guide RNA and/or the Cas endonuclease. Tethered DNAs can allow for co-localizing target and template DNA, useful in genome editing and targeted genome regulation, and can also be useful in targeting post-mitotic cells where function of endogenous HR machinery is expected to be highly diminished (Mali et al. 2013 Nature Methods Vol. 10:957-963.) The polynucleotide modification template may be present transiently in the cell or it can be introduced via a viral replicon.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

In addition to modification by a double strand break technology, modification of one or more bases without such double strand break are achieved using base editing technology, see e.g., Gaudelli et al., (2017) Programmable base editing of A*T to G*C in genomic DNA without DNA cleavage. Nature 551 (7681):464-471; Komor et al., (2016) Programmable editing of a target base in genomic DNA without double-stranded DNA cleavage, Nature 533 (7603):420-4.

These fusions contain dCas9 or Cas9 nickase and a suitable deaminase, and they can convert e.g., cytosine to uracil without inducing double-strand break of the target DNA. Uracil is then converted to thymine through DNA replication or repair. Improved base editors that have targeting flexibility and specificity are used to edit endogenous locus to create target variations and improve grain yield. Similarly, adenine base editors enable adenine to inosine change, which is then converted to guanine through repair or replication. Thus, targeted base changes i.e., C·G to T·A conversion and A·T to G·C conversion at one or more locations are made using appropriate site-specific base editors.

In an embodiment, base editing is a genome editing method that enables direct conversion of one base pair to another at a target genomic locus without requiring double-stranded DNA breaks (DSBs), homology-directed repair (HDR) processes, or external donor DNA templates. In an embodiment, base editors include (i) a catalytically impaired CRISPR-Cas9 mutant that are mutated such that one of their nuclease domains cannot make DSBs; (ii) a single-strand-specific cytidine/adenine deaminase that converts C to U or A to G within an appropriate nucleotide window in the single-stranded DNA bubble created by Cas9; (iii) a uracil glycosylase inhibitor (UGI) that impedes uracil excision and downstream processes that decrease base editing efficiency and product purity; and (iv) nickase activity to cleave the non-edited DNA strand, followed by cellular DNA repair processes to replace the G-containing DNA strand.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

TAL effector nucleases (TALEN) are a class of sequence-specific nucleases that can be used to make double-strand breaks at specific target sequences in the genome of a plant or other organism. (Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognized a sequence of 9 contiguous nucleotides, with a dimerization requirement of the nuclease, two sets of zinc finger triplets are used to bind an 18 nucleotide recognition sequence.

Genome editing using DSB-inducing agents, such as Cas9-gRNA complexes, has been described, for example in U.S. Patent Application US 2015-0082478 A1, published on Mar. 19, 2015, WO2015/026886 A1, published on Feb. 26, 2015, WO2016007347, published on Jan. 14, 2016, and WO201625131, published on Feb. 18, 2016, all of which are incorporated by reference herein.

The term “Cas gene” herein refers to a gene that is generally coupled, associated or close to, or in the vicinity of flanking CRISPR loci in bacterial systems. The terms “Cas gene”, “CRISPR-associated (Cas) gene” are used interchangeably herein. The term “Cas endonuclease” herein refers to a protein encoded by a Cas gene. A Cas endonuclease herein, when in complex with a suitable polynucleotide component, is capable of recognizing, binding to, and optionally nicking or cleaving all or part of a specific DNA target sequence. A Cas endonuclease described herein comprises one or more nuclease domains. Cas endonucleases of the disclosure includes those having a HNH or HNH-like nuclease domain and/or a RuvC or RuvC-like nuclease domain. A Cas endonuclease of the disclosure includes any polynucleotide-guided endonuclease such as Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, and homologs or modified versions thereof, Argonaute and homologs or modified versions thereof.

As used herein, the terms “guide polynucleotide/Cas endonuclease complex”, “guide polynucleotide/Cas endonuclease system”, “guide polynucleotide/Cas complex”, “guide polynucleotide/Cas system”, “guided Cas system” are used interchangeably herein and refer to at least one guide polynucleotide and at least one Cas endonuclease that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double strand break) the DNA target site. A guide polynucleotide/Cas endonuclease complex herein can comprise Cas protein(s) and suitable polynucleotide component(s) of any of the four known CRISPR systems (Horvath and Barrangou, 2010, Science 327:167-170) such as a type I, II, or III CRISPR system. A Cas endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas protein. Such recognition and cutting of a target sequence by a Cas endonuclease typically occurs if the correct protospacer-adjacent motif (PAM) is located at or adjacent to the 3′ end of the DNA target sequence. Alternatively, a Cas protein herein may lack DNA cleavage or nicking activity, but can still specifically bind to a DNA target sequence when complexed with a suitable RNA component. (See also U.S. Patent Application US 2015-0082478 A1, published on Mar. 19, 2015 and US 2015-0059010 A1, published on Feb. 26, 2015, both are hereby incorporated in its entirety by reference).

A guide polynucleotide/Cas endonuclease complex can cleave one or both strands of a DNA target sequence. A guide polynucleotide/Cas endonuclease complex that can cleave both strands of a DNA target sequence typically comprise a Cas protein that has all of its endonuclease domains in a functional state (e.g., wild type endonuclease domains or variants thereof retaining some or all activity in each endonuclease domain). Non-limiting examples of Cas9 nickases suitable for use herein are disclosed in U.S. Patent Appl. Publ. No. 2014/0189896, which is incorporated herein by reference.

Other Cas endonuclease systems have been described in PCT patent applications PCT/US16/32073, filed May 12, 2016 and PCT/US16/32028 filed May 12, 2016, both applications incorporated herein by reference.

“Cas9” (formerly referred to as Cas5, Csn1, or Csx12) herein refers to a Cas endonuclease of a type II CRISPR system that forms a complex with a crNucleotide and a tracrNucleotide, or with a single guide polynucleotide, for specifically recognizing and cleaving all or part of a DNA target sequence. Cas9 protein comprises a RuvC nuclease domain and an HNH (H—N—H) nuclease domain, each of which can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al, Cell 157:1262-1278). A type II CRISPR system includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA.

Any guided endonuclease can be used in the methods disclosed herein. Such endonucleases include, but are not limited to Cas9 and Cpf1 endonucleases. Many endonucleases have been described to date that can recognize specific PAM sequences (see for example—Jinek et al. (2012) Science 337 p 816-821, PCT patent applications PCT/US16/32073, filed May 12, 2016 and PCT/US16/32028 filed May 12, 2016 and Zetsche B et al. 2015. Cell 163, 1013) and cleave the target DNA at a specific position. It is understood that based on the methods and embodiments described herein utilizing a guided Cas system one can now tailor these methods such that they can utilize any guided endonuclease system.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site.

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site. A guide RNA/Cas endonuclease complex herein can comprise Cas protein(s) and suitable RNA component(s) of any of the four known CRISPR systems (Horvath and Barrangou, 2010, Science 327:167-170) such as a type I, II, or III CRISPR system. A guide RNA/Cas endonuclease complex can comprise a Type II Cas9 endonuclease and at least one RNA component (e.g., a crRNA and tracrRNA, or a gRNA). (See also U.S. Patent Application US 2015-0082478 A1, published on Mar. 19, 2015 and US 2015-0059010 A1, published on Feb. 26, 2015, both are hereby incorporated in its entirety by reference).

The guide polynucleotide can be introduced into a cell transiently, as single stranded polynucleotide or a double stranded polynucleotide, using any method known in the art such as, but not limited to, particle bombardment, Agrobacterium transformation or topical applications. The guide polynucleotide can also be introduced indirectly into a cell by introducing a recombinant DNA molecule (via methods such as, but not limited to, particle bombardment or Agrobacterium transformation) comprising a heterologous nucleic acid fragment encoding a guide polynucleotide, operably linked to a specific promoter that is capable of transcribing the guide RNA in said cell. The specific promoter can be, but is not limited to, a RNA polymerase III promoter, which allow for transcription of RNA with precisely defined, unmodified, 5′- and 3′-ends (DiCarlo et al., Nucleic Acids Res. 41:4336-4343; Ma et al., Mol. Ther. Nucleic Acids 3: e161) as described in WO2016025131, published on Feb. 18, 2016, incorporated herein in its entirety by reference.

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, or any other DNA molecule in the genome (including chromosomal, choloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. Cells include, but are not limited to, human, non-human, animal, bacterial, fungal, insect, yeast, non-conventional yeast, and plant cells as well as plants and seeds produced by the methods described herein. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

Methods for “modifying a target site” and “altering a target site” are used interchangeably herein and refer to methods for producing an altered target site.

The length of the target DNA sequence (target site) can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other Cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by an Cas endonuclease. Assays to measure the single or double-strand break of a target site by an endonuclease are known in the art and generally measure the overall activity and specificity of the agent on DNA substrates containing recognition sites.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

The terms “targeting”, “gene targeting” and “DNA targeting” are used interchangeably herein. DNA targeting herein may be the specific introduction of a knock-out, edit, or knock-in at a particular DNA sequence, such as in a chromosome or plasmid of a cell. In general, DNA targeting can be performed herein by cleaving one or both strands at a specific DNA sequence in a cell with an endonuclease associated with a suitable polynucleotide component. Such DNA cleavage, if a double-strand break (DSB), can prompt NHEJ or HDR processes which can lead to modifications at the target site.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide an guide polynucleotide/Cas endonuclease complex to a unique DNA target site.

The terms “knock-out”, “gene knock-out” and “genetic knock-out” are used interchangeably herein. A knock-out represents a DNA sequence of a cell that has been rendered partially or completely inoperative by targeting with a Cas protein; such a DNA sequence prior to knock-out could have encoded an amino acid sequence, or could have had a regulatory function (e.g., promoter), for example. A knock-out may be produced by an indel (insertion or deletion of nucleotide bases in a target DNA sequence through NHEJ), or by specific removal of sequence that reduces or completely destroys the function of sequence at or near the targeting site.

The guide polynucleotide/Cas endonuclease system can be used in combination with a co-delivered polynucleotide modification template to allow for editing (modification) of a genomic nucleotide sequence of interest. (See also U.S. Patent Application US 2015-0082478 A1, published on Mar. 19, 2015 and WO2015/026886 A1, published on Feb. 26, 2015, both are hereby incorporated in its entirety by reference.)

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (by HR, wherein a suitable donor DNA polynucleotide is also used). Examples of knock-ins are a specific insertion of a heterologous amino acid coding sequence in a coding region of a gene, or a specific insertion of a transcriptional regulatory element in a genetic locus.

Various methods and compositions can be employed to obtain a cell or organism having a polynucleotide of interest inserted in a target site for a Cas endonuclease. Such methods can employ homologous recombination to provide integration of the polynucleotide of Interest at the target site. In one method provided, a polynucleotide of interest is provided to the organism cell in a donor DNA construct. As used herein, “donor DNA” is a DNA construct that comprises a polynucleotide of Interest to be inserted into the target site of a Cas endonuclease. The donor DNA construct further comprises a first and a second region of homology that flank the polynucleotide of Interest. The first and second regions of homology of the donor DNA share homology to a first and a second genomic region, respectively, present in or flanking the target site of the cell or organism genome. By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

The amount of sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some embodiments the regions of homology share significant sequence homology to the genomic sequence immediately flanking the target site, it is recognized that the regions of homology can be designed to have sufficient homology to regions that may be further 5′ or 3′ to the target site. In still other embodiments, the regions of homology can also have homology with a fragment of the target site along with downstream genomic regions. In one embodiment, the first region of homology further comprises a first fragment of the target site and the second region of homology comprises a second fragment of the target site, wherein the first and second fragments are dissimilar.

As used herein, “homologous recombination” includes the exchange of DNA fragments between two DNA molecules at the sites of homology.

Further uses for guide RNA/Cas endonuclease systems have been described (See U.S. Patent Application US 2015-0082478 A1, published on Mar. 19, 2015, WO2015/026886 A1, published on Feb. 26, 2015, US 2015-0059010 A1, published on Feb. 26, 2015, U.S. application 62/023,246, filed on Jul. 7, 2014, and U.S. application 62/036,652, filed on Aug. 13, 2014, all of which are incorporated by reference herein) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Methods for transforming dicots, primarily by use of Agrobacterium tumefaciens, and obtaining transgenic plants have been published, among others, for cotton (U.S. Pat. Nos. 5,004,863, 5,159,135); soybean (U.S. Pat. Nos. 5,569,834, 5,416,011); Brassica (U.S. Pat. No. 5,463,174); peanut (Cheng et al., Plant Cell Rep. 15:653-657 (1996), McKently et al., Plant Cell Rep. 14:699-703 (1995)); papaya (Ling et al., Bio/technology 9:752-758 (1991)); and pea (Grant et al., Plant Cell Rep. 15:254-258 (1995)). For a review of other commonly used methods of plant transformation see Newell, C. A., Mol. Biotechnol. 16:53-65 (2000). One of these methods of transformation uses Agrobacterium rhizogenes (Tepfler, M. and Casse-Delbart, F., Microbiol. Sci. 4:24-28 (1987)). Transformation of soybeans using direct delivery of DNA has been published using PEG fusion (PCT Publication No. WO 92/17598), electroporation (Chowrira et al., Mol. Biotechnol. 3:17-23 (1995); Christou et al., Proc. Natl. Acad. Sci. U.S.A. 84:3962-3966 (1987)), microinjection, or particle bombardment (McCabe et al., Biotechnology 6:923-926 (1988); Christou et al., Plant Physiol. 87:671-674 (1988)).

There are a variety of methods for the regeneration of plants from plant tissues. The particular method of regeneration will depend on the starting plant tissue and the particular plant species to be regenerated. The regeneration, development and cultivation of plants from single plant protoplast transformants or from various transformed explants is well known in the art (Weissbach and Weissbach, Eds.; In Methods for Plant Molecular Biology; Academic Press, Inc.: San Diego, CA, 1988). This regeneration and growth process typically includes the steps of selection of transformed cells, culturing those individualized cells through the usual stages of embryonic development or through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

This disclosure also concerns a method of altering (increasing or decreasing) the expression of at least one heterologous nucleic acid fragment in a plant cell which comprises:
(a) transforming a plant cell with the recombinant expression construct described herein;
(b) growing fertile mature plants from the transformed plant cell of step (a);
(c) selecting plants containing a transformed plant cell wherein the expression of the heterologous nucleic acid fragment is increased or decreased.

Transformation and selection can be accomplished using methods well-known to those skilled in the art including, but not limited to, the methods described herein.

TABLE 2
Stature modification guide RNAs and target edit description

| | | | Relative | Relative | |
|---|---|---|---|---|---|
| Target | | Start | End | DSB | |
| Sequence | Target Sequence (5′-3′) and | Position | Position | Position | Description of |
| Designation | target strand | (bp) | (bp) | (bp) | Edit |
| ZM-BR1- | GGATCAGCAAGCGCCCC- | 371 | 387 | 373/374 | Insertion of |
| CR3 (SEQ ID | Complementary | | | | Mutag |
| NO: 34) | | | | | sequence (re- |
| | | | | | creation of br1- |
| | | | | | Mutag allele) at |
| | | | | | position 373 of |
| | | | | | the CDS (by |
| | | | | | sgRNA) |
| ZM-BR1- | GCTGCGCACCGCTTCTA-Sense | 418 | 434 | 431/432 | Deletion or |
| CR4 (SEQ ID | | | | | Frameshift at |
| NO: 36) | | | | | position 431 of |
| | | | | | the CDS |
| ZM-BR1- | GCGTTCATAGATTTCCTC- | 1114 | 1131 | 1116/1117 | Deletion or |
| CR6 (SEQ ID | Complementary | | | | Frameshift at |
| NO: 37) | | | | | position 1116 of |
| | | | | | the CDS |
| ZM-BR1- | Deletion by dual gRNAs (listed | 432 | 1116 | see above | Deletion from |
| CR4 + ZM- | above) | | | | position 432 to |
| BR1-CR6 | | | | | 1116 of the CDS |
| | | | | | (685 bp of CDS); |
| | | | | | frameshift |
| | | | | | INDELs also |
| | | | | | targeted at CR |
| | | | | | sites (by dual |
| | | | | | gRNA) |
| ZM-BR1- | CCTCTTCTGTGACGAGGTTA-Sense | 2905 | 2924 | 2921/2922 | INDELs |
| CR7 (SEQ ID | | | | | (SNPs/deletions) |
| NO: 38) | | | | | at position 2921 |
| | | | | | of PRO |
| ZM-BR1- | TGGAGACACAATAATGTCGC-Sense | 4120 | 4139 | 4136/4137 | INDELs |
| CR8 (SEQ ID | | | | | (SNPs/deletions) |
| NO: 39) | | | | | at position 4136 |
| | | | | | of PRO |
| ZM-BR1- | AGTTCCGTGCCTTGCACTTC- | 5544 | 5563 | 5546/5547 | INDELs |
| CR9 (SEQ ID | Complementary | | | | (SNPs/deletions) |
| NO: 40) | | | | | at position 5546 |
| | | | | | of PRO |
| ZM-BR1- | Deletion by dual gRNAs (listed | 2922 | 4136 | see above | Deletion from |
| CR7 + ZM- | above) | | | | position 2922 to |
| BR1-CR8 | | | | | 4136 of the PRO |
| | | | | | (1215 bp) |
| ZM-BR1- | Deletion by dual gRNAs (listed | 4137 | 5546 | see above | Deletion from |
| CR8 + ZM- | above) | | | | position 4137 to |
| BR1-CR9 | | | | | 5546 of the PRO |
| | | | | | (1410 bp) |
| ZM-BR2- | GATCGACCGCAAGACGG-Sense | 3228 | 3304 | 3301/3302 | Deletion or |
| CR1 (SEQ ID | | | | | Frameshift at |
| NO: 41) | | | | | position 3301 of |
| | | | | | the CDS |
| ZM-BR2- | AGTCGGAGCGGTGCGTGC- | 3878 | 3895 | 3880/3881 | Deletion or |
| CR3 (SEQ ID | Complementary | | | | Frameshift at |
| NO: 42) | | | | | position 3880 of |
| | | | | | the CDS |
| ZM-BR2- | Deletion by dual gRNAs (listed | 3302 | 3880 | see above | Deletion from |
| CR1 + ZM- | above) | | | | position 3302 to |
| BR2-CR3 | | | | | 3880 of the CDS |
| | | | | | (579 bp of CDS); |
| | | | | | removes |
| | | | | | transporter |
| | | | | | domain; |
| | | | | | frameshift |
| | | | | | INDELs also |
| | | | | | targeted at CR |
| | | | | | sites (by dual |
| | | | | | gRNA) |
| ZM-BR2- | ACGTGCGCAAGTACAACCTG-Sense | 3557 | 3576 | 3573/3574 | Alter Arg 1193 |
| CR4 (SEQ ID | | | | | to Leucine with |
| NO: 46) | | | | | oligo template |
| | | | | | (Xing et al, |
| | | | | | 2015) |
| ZM-BR2- | AGTACAACCTGCGGGCGCTG-Sense | 3566 | 3585 | 3582/3583 | Alternate |
| CR5 (SEQ ID | | | | | approach to |
| NO: 47) | | | | | alter Arg 1193 |
| | | | | | to Leucine with |
| | | | | | oligo template |
| | | | | | (Xing et al, |
| | | | | | 2015) |
| ZM-BR2- | CGTGCGCAAGTACAACCTGC-Sense | 3558 | 3577 | 3574/3575 | SDN1 Deletions |
| CR6 (SEQ ID | | | | | and/or point |
| NO: 48) | | | | | mutations at |
| | | | | | location of |
| | | | | | R1193 (known |
| | | | | | to affect |
| | | | | | stature) |
| ZM-BR2- | TGCTGGACGGGCACGACCTC-Sense | 1562 | 1581 | 1578/1579 | SDN1 Deletions |
| CR7 (SEQ ID | | | | | and/or point |
| NO: 49) | | | | | mutations in |
| | | | | | first ABC |
| | | | | | transporter |
| | | | | | domain |
| ZM-BR2- | CGCCATGCTCAAGAACCC- | 1842 | 1859 | 1844/1845 | SDN1 Deletions |
| CR8 (SEQ ID | Complementary | | | | and/or point |
| NO: 50) | | | | | mutations in |
| | | | | | conserved ABC |
| | | | | | binding site |
| ZM-BR2- | GGTTCGACGCGGACGAGAAC- | 2663 | 2682 | 2665/2666 | SDN1 Deletions |
| CR9 (SEQ ID | Complementary | | | | and/or point |
| NO: 51) | | | | | mutations in |
| | | | | | transmembrane |
| | | | | | domain |
| ZM-BR2- | GGTGTTCCGCGACCTGAGCC- | 3411 | 3430 | 3413/3414 | SDN1 Deletions |
| CR10 (SEQ | Complementary | | | | and/or point |
| ID NO: 52) | | | | | mutations in |
| | | | | | second ABC |
| | | | | | transporter |
| | | | | | domain |
| ZM-BR2- | GAACGCGCACCGGTTCATCG-Sense | 3708 | 3727 | 3724/3725 | SDN1 Deletions |
| CR11 (SEQ | | | | | and/or point |
| ID NO: 53) | | | | | mutations in |
| | | | | | ATPase domain |
| ZM-BR2- | GTTGGACTCTTCTACTGCTA- | 696 | 715 | 698/699 | SDN1 |
| CR12 (SEQ | Complementary | | | | SNP/INDELs- |
| ID NO: 54) | | | | | within 5′UTR |
| ZM-BR2- | TGCCACTCTGCTGAGGTGGG-Sense | 880 | 899 | 896/897 | SDN1 |
| CR13 (SEQ | | | | | SNP/INDELs- |
| ID NO: 55) | | | | | within 5′UTR |
| ZM-BR2- | GTATCGCGAGATGCTTATTT- | not in | not in | not in B73 | SDN1 |
| CR14 (SEQ | Complementary | B73 | B73 | | SNP/INDELs |
| ID NO: 56) | | | | | |
| ZM-BR2- | AGCAGCATTAACCGAGTGAA-Sense | not in | not in | not in B73 | SDN1 |
| CR15 (SEQ | | B73 | B73 | | SNP/INDELs |
| ID NO: 57) | | | | | |
| ZM-BR2- | CAGAGTGCAGGACATAACTC-Sense | not in | not in | not in B73 | SDN1 |
| CR16 (SEQ | | B73 | B73 | | SNP/INDELs |
| ID NO: 58) | | | | | |
| ZM-BR2- | GGACAAATTGAACCTGGAAC | not in | not in | not in B73 | SDN1 |
| CR17 (SEQ | | B73 | B73 | | SNP/INDELs |
| ID NO: 59) | | | | | |
| ZM-BR2- | CATGCATCCATTCCCATTCG- | 613 | 632 | 615/616 | SDN1 |
| CR18 (SEQ | Complementary | | | | SNP/INDELs |
| ID NO: 60) | | | | | |
| ZM-BR2- | CATGCATCCATTCCCATTCG- | 699 | 896 | see above | Deletion from |
| CR12 + ZM- | Complementary | | | | position 699 to |
| BR1-CR13 | | | | | 896 of the |
| (SEQ ID NO: | | | | | 5′UTR within |
| 61) | | | | | PRO (184 bp in |
| | | | | | B73); INDELs |
| | | | | | also targeted at |
| | | | | | CR sites (by dual |
| | | | | | gRNA) |
| ZM-BR2- | Deletion by dual gRNAs (listed | not in | not in | not in 673 | |
| CR14 + ZM- | above) | B73 | B73 | | |
| BR1-CR15 | | | | | |
| ZM-BR2- | Deletion by dual gRNAs (listed | not in | not in | not in B73 | |
| CR15 + ZM- | above) | B73 | B73 | | |
| BR1-CR16 | | | | | |
| ZM-BR2- | Deletion by dual gRNAs (listed | not in | not in | not in B73 | |
| CR16 + ZM- | above) | B73 | B73 | | |
| BR1-CR17 | | | | | |
| ZM-BR2- | Deletion by dual gRNAs (listed | not in | not in | not in B73 | |
| CR17 + ZM- | above) | B73 | B73 | | |
| BR1-CR18 | | | | | |
| ZM-BR2- | Deletion by dual gRNAs (listed | not in | not in | not in B73 | |
| CR14 + ZM- | above) | B73 | B73 | | |
| BR1-CR18 | | | | | |
| ZM-D8-CR2 | TTATTAGCTGGCTAGCTAGGC- | 405 | 425 | 407/408 | Deletion or |
| (SEQ ID NO: | Complementary | | | | INDEL at |
| 62) | | | | | position 407 of |
| | | | | | the GENE |
| ZM-D8-CR3 | TCCACGGACTCGGCGCGGGAGC- | 961 | 982 | 963/964 | Deletion or |
| (SEQ ID NO: | Complementary | | | | Frameshift at |
| 63) | | | | | position 963 of |
| | | | | | the CDS (in |
| | | | | | DELLA domain) |
| ZM-D8-CR2 | Deletion by dual gRNAs (listed | 408 | 963 | see above | Deletion from |
| CR2 + ZM- | above) | | | | position 408 to |
| D8-CR3 | | | | | 963 of the gene |
| | | | | | (556 bp of |
| | | | | | gene); removes |
| | | | | | N-terminal |
| | | | | | DELLA domain |
| | | | | | (by dual gRNA) |
| ZM-D8-CR4 | CCTACTTCGGCGAGGCGCTTGC- | 1295 | 1316 | 1297/1298 | Deletion or |
| (SEQ ID NO: | Complementary | | | | frameshift at |
| 64) | | | | | position 1297 of |
| | | | | | the GENE in NLS |
| | | | | | region of |
| | | | | | dimerization |
| | | | | | domain |
| ZM-D8-CRS | CGTGTATCGCTTCCGCC- | 1323 | 1339 | 1325/1326 | Deletion or |
| (SEQ ID NO: | Complementary | | | | frameshift at |
| 65) | | | | | position 1325 of |
| | | | | | the GENE in NLS |
| | | | | | region of |
| | | | | | dimerization |
| | | | | | domain |
| ZM-D8- | Deletion by dual gRNAs (listed | 1298 | 1325 | see above | Deletion from |
| CR4 + ZM- | above) | | | | position 1298 to |
| D8-CR5 | | | | | 1325 of the |
| | | | | | gene (28 bp of |
| | | | | | gene); removes |
| | | | | | NLS sequence |
| | | | | | and portion of |
| | | | | | dimerization |
| | | | | | domain (by dual |
| | | | | | gRNA) |
| ZM-D8-CR6 | CTACCTGAAGTTCGCCC- | 1413 | 1429 | 1415/1416 | Deletion or |
| (SEQ ID NO: | Complementary | | | | frameshift at |
| 66) | | | | | position 1415 of |
| | | | | | the GENE in |
| | | | | | VHIID motif |
| ZM-D8-CR7 | GTTCGCGCACACCATCCGCGTGGAC- | 1647 | 1671 | 1649/1650 | Deletion or |
| (SEQ ID NO: | Complementary | | | | frameshift at |
| 67) | | | | | position 1649 of |
| | | | | | the GENE in C- |
| | | | | | terminal GRAS |
| | | | | | domain |
| ZM-D8- | Deletion by dual gRNAs (listed | 1416 | 1649 | see above | Deletion from |
| CR6 + ZM- | above) | | | | position 1416 to |
| D8-CR7 | | | | | 1649 of the |
| | | | | | gene (234 bp of |
| | | | | | gene); removes |
| | | | | | VHIID motif and |
| | | | | | portion of GRAS |
| | | | | | domain by dual |
| | | | | | gRNA) |
| ZM-D8-CR8 | GAGGGCGATGACACGGATGAC- | 1735 | 1755 | 1737/1738 | Deletion or |
| (SEQ ID NO: | Complementary | | | | frameshift at |
| 68) | | | | | position 1755 of |
| | | | | | the GENE in the |
| | | | | | in C-terminal |
| | | | | | GRAS domain |
| ZM-D8-CR9 | GGTCATGTCGGAGGTGTAC- | 2034 | 2052 | 2036/2037 | Deletion or |
| (SEQ ID NO: | Complementary | | | | frameshift at |
| 69) | | | | | position 2036 of |
| | | | | | the GENE in the |
| | | | | | in C-terminal |
| | | | | | GRAS domain |
| ZM-D8- | Deletion by dual gRNAs (listed | 1738 | 2036 | see above | Deletion from |
| CR8 + ZM- | above) | | | | position 1738 to |
| D8-CR9 | | | | | 2036 of the |
| | | | | | gene (299 bp of |
| | | | | | gene); removes |
| | | | | | SH2 motif and |
| | | | | | LXXLL motif in C- |
| | | | | | terminal GRAS |
| | | | | | domain (by dual |
| | | | | | gRNA) |

TABLE 3
D8 genomic target edit description

| Example of editing | | |
|---|---|---|
| rationale | Description of genome edits | Target Position |
| Delete DELLA domain | In frame deletion or out-of-frame edit | 577-768 in D8 |
| (larger designated region) | | B73 gene |
| Delete DELLA domain | In frame deletion or out-of-frame edit | 553-603 in D8 |
| (smaller specified | | B73 gene |
| domain) | | |
| N-terminal truncation(s) | Various size deletions of N-terminus into DELLA | |
| | domain - translation would then initiate at | |
| | endogenous MET codons within D8 (such as at | |
| | amino acid 15, 22, 23, 53, 65, 67, 69, 106, 184, 201, | |
| | etc.). Dwarf D8MPL initiates at Methionine 106. | |
| | An edit creating a stop/frameshift after D8's native | |
| | start creates translation initation at a subseuqent | |
| | Met codon. | |
| Re-create D8-1 deln | 5 amino acid deletion in larger DELLA domain | 604-618 in D8 |
| | | B73 gene |
| Re-create D8 MUT | 4 amino acid deletion in larger DELLA domain | 607-618 in D8 |
| | | B73 gene |
| Re-create D8-2023 | 12 amino acid deletion in larger DELLA domain | 700-735 in D8 |
| | | B73 gene |
| Re-create D8-1023 | 2 amino acid deletion in larger DELLA domain | 745-750 in D8 |
| | | B73 gene |
| Delete VHYNP motif | Delete/alter VHYNP domain which appears in | 715-729 in D8 |
| | larger DELLA domain | B73 gene |
| Delete dimerization | Delete/alter dimerization domain which appears at | 1168-1434 of |
| domain | beginning of GRAS domain (D8 functions as dimer) | D8 B73 gene |
| Delete VHIID domain | Delete/alter VHIID motif which appears in GRAS | 1387-1584 of |
| | domain | D8 B73 gene |
| Delete SH2 domain | Remove/alter SH2 domain hindering protein's | 1855-1971 of |
| | ability to bind or “dock” to phosphorylated | D8 B73 gene |
| | tyrosine residues on other proteins. (SH2 motif | |
| | within larger GRAS domain) | |
| Delete NLS domain | Nuclear localization signal exists within larger GRAS | 1285-1388 of |
| | domain in D8 (the C-terminal half of gene). | D8 B73 gene |
| | Deletion of Partitioning/compartmentalization of | |
| | gene to alter stature. | |
| Delete LXXLL motif(s) | Alter/remove LXXLL binding motif referred to as an | 1st: 1168- |
| | NR (nuclear receptor) box - coactivators - inframe | 1185;2nd: |
| | and/or out-of-frame deletions may be tested. | 1792-1806 of |
| | (LXXLL motifs within larger GRAS domain) | D8 B73 gene |
| GRAS domain variation | Alterations may affect flowering as well as stature - | 1168-2292 of |
| | in-frame and/or out-of-frame deletions may | D8 B73 gene |
| | cause phenotype | |
| Promoter swap | Tissue specific or preferred expression (e.g. | |
| | targeted to stalk so reduced cell expansion | |
| | limitedly impacts other tissues)/ Gene Regulation | |
| | controlled temporally (e.g. in developing juvenile | |
| | cells) to reduce expansion and thus stature | |
| Enhancer insertion | Increase expression of native peptide | |

### EXAMPLES

The present disclosure is further defined in the following Examples. From the above discussion and these Examples, one skilled in the art can ascertain the essential characteristics of this disclosure, and without departing from the spirit and scope thereof, can make various changes and modifications of the disclosure to adapt it to various usages and conditions. Thus, various modifications of the disclosure in addition to those shown and described herein will be apparent to those skilled in the art from the foregoing description. Such modifications are also intended to fall within the scope of the appended claims.

The disclosure of each reference set forth herein is incorporated herein by reference in its entirety.

### Example 1

### Isolation and Characterization of a Br1 Mutant in a Monocot Plant

A semi-dwarf mutant was isolated from a F2 population of a Mutator crossed with an elite line. The semi-dwarf mutant was crossed with br1, br2, and br3 known mutations which are insensitive to GA3. The F1s of both br2 and br3 mutations with semi-dwarf mutant were normal in plant height. This phenotype indicated non-allelic nature of the semi-dwarf mutant with both br2 and br3 mutations. However, the semi-dwarf mutant in reciprocal crosses with br1-CooP could not complement each other and the F1s had reduced plant height indicating that the semi-dwarf mutant is a weak allele of br1 locus and thus named as br1-Mutag.

The br1-ref mutant allele was introgressed in B73 background and was subjected to plant height measurements post flowering just before harvesting. The br1-ref mutant allele was a stronger allele and showed around 50% height reduction as compared to its WT-sibs in BC3 generation. Compared to the br2 mutation where the lower internodes' length is reduced significantly, in one of the identified br1 mutants, most internodes were slightly shorter than its WT-sibs. Similarly, the total plant height and car height in homozygous br1-Mutag generation were measured in BC4F2. The br1-Mutag plants were 30 inches shorter than its heterozygous and homozygous wild-type sibs in total plant height and 10 inches in ear height. On an average, the numbers of both nodes and internodes below car in br1-Mutag was one less than its WT-sibs.

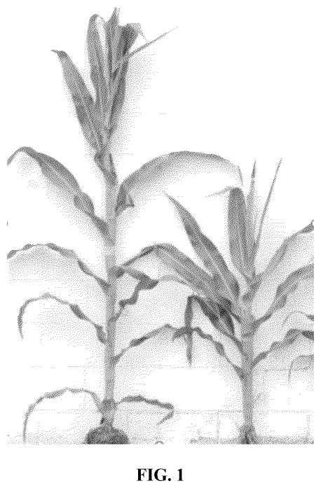

The dwarf phenotype of br1 mutant became evident at around 5th week stage, thus, samples for histology were collected from stalks at v7-v8 stage of plants. Middle section of 4th internode of both homozygous br1-ref mutant and its WT-sib was used for collecting stalk samples. Light microscopy analysis was performed to investigate the cause of observed height reduction in br1 mutant using auto florescence under confocal microscopy. Differences were observed in cell length in longitudinal section and cell numbers in both rind and pith cross sections. In order to quantify these differences, data from more than 1000 cells each from mutant and WT-sib were taken. Total of 101 images from 4 mutants and 4 WT-sibs from 4th internodes were screened using MetaMorph (Molecular Devices, Sunnyvale, CA) image analysis software using 700×700 um square areas) for measuring cell length and calculating cell counts. As expected the average cell length in br1 mutant was reduced significantly (131.13+/−1.01 um) as compared to 149.94+/−1.02 um in its WT-sib (p=<0.01) whereas the cell count was increased slightly from 24.49+/−1.8 in WT-sib to 27.21+/−1.7 in br1 mutant (p=<0.01). Taken together, our findings indicated that brachytic 1 mutation reduced cell length and increased cell counts in br1 mutant without changing its stalk diameter as compared to its WT-sib (FIGS. 1A & 1D).

### Example 2

### Cloning and Validation of a Candidate Gene for Br1 Locus

DNAs from the segregating br1-Mutag mutants along with its WT-sibs in BC2F2 were subjected to co-segregation analysis. Co-segregation analysis was performed first on pool DNAs from 8 mutants and 8 wild-types by digesting DNA with two four base cutter restriction enzymes and ligating with an adapter. PCR based approach SAIFF (Sequence Amplified Insertion Flanking Fragments) was followed by using adapter and Mu-TIR (terminal inverted repeat) primer and their nested primers. Database searches using co-segregating PCR fragment sequence as query against Pioneer Sequence database revealed a full length corn EST that exhibited 100% identity and annotated as a Transcription Regulator HTH, MYB-type DNA binding protein (SEQ ID NO: 5), homologous to Myb105 in Arabidopsis (SEQ ID NOS: 17 and 18) was identified as a putative candidate gene. A complete genomic sequence corresponding to this EST was also found from BAC sequences of both A63 and Mo17 inbred lines and it was named as ZmBr1. Gene specific primers (GSPs) were designed from the ZmBr1 genomic sequence and used to extend linkage analysis using 208 BC2F2 plants comprising of 110 mutants and 98 wild-type plants. No recombinants were found between the genotype and semi-dwarf phenotype of br1-Mutag mutation, suggesting that the two were tightly linked. The Mu-insertion in br1-Mutag mutant allele was found in intron1, 15 bp from the intron1 and exon2 junction.

Two mutant alleles showed allelism in crosses with the br1-ref allele and here named as br1-3 and br1-4. DNA from these two new mutant alleles along with br1-ref allele was subjected to a reverse-genetics (RG) using PCR-amplification and Southern blot analysis. For RG, the ZmBr1-gene specific primers (GSPs) were used in combination with Mutator-terminal inverted repeat (Mu-TIR) primer in PCR reactions using template DNAs from the new mutant alleles. None of these three mutants yielded any PCR product when GSP1 and GSP5 (SEQ ID NOS: 22 and 26) and GSP4 and GSP6 (SEQ ID NOS: 25 and 27) were used in combination with Mu-TIR primer (SEQ ID NO: 30). However, GSP1+GSP5 amplified the same size PCR product in all three mutants and WT-sib of br1-3 (which was isolated from EMS population) for the whole length of Br1 gene. A combination of GSP4+GSP6 could not amplify exon3 in br1-ref allele as compared all other mutants and their WT-sibs. Furthermore, the sequencing of PCR products of br1-3 mutant allele revealed a base pair change leading to one amino acid change in exon2 as compared to A63 sequence. Southern blot analysis using FL-cDNA of Br1 gene as probe detected polymorphism in br1-ref mutant allele as compared to A63 with both Eco RI and Hind III restriction enzymes and complete deletion of Br1 gene in br1-4 mutant as compared to its progenitor P8. Further by using Illumina 4k markers it was determined that the br1-4 mutant allele has a large deletion of 0.46 cM in size. The 3.5 kb/Hind III restriction fragment was excised from br1-ref, purified DNA was self-ligated to re-circularize and amplified by GSPs from exon3 using Inverse PCR (IPCR). Cloning and sequencing of IPCR product revealed that the br1-ref. mutant allele has insertion of a novel retro-transposon element (RTE) in exon3. A complete 2.8 kb sequence of this novel RTE is listed in (SEQ ID NO: 15). The novel RTE has 320 bp terminal inverted repeat (TIR; underline sequence) with 3 bp direct duplication flanking both TIRs at the site of insertion. Taken together these findings where four insertions in the same gene at different sites leading to a brachytic mutation clearly established that Br1 gene/allele is responsible for br1 mutant phenotype.

For candidate gene validation, PCR, Southern blot, and RT-PCR analyses were used to validate the candidate gene for br1 mutation. Gene specific primers (GSPs) could not amplify exon3 in br1-CooP due to the presence of RTE in it whereas the br1-EMS allele has similar size PCR product but a base pair change leading to one aa change in exon2 was detected as compared to A63 sequence. Southern blot analysis using FL-cDNA of Br1 gene as probe showed complete deletion of Br1 gene in br1-P8 mutant as compared to its progenitor P8. The br1-Coop mutant allele showed polymorphism with both Eco RI and Hind III restriction enzymes. RT-PCR analysis showed a complete absence of Br1-transcript in br1-CooP mutant indicating that the br1-Coop is a null allele whereas a bigger size transcript in br1-Mutag as compared to its WT-sibs indicated the cause of a weak phenotype. Cloning and sequencing of RT-PCR product in br1-Mutag allele indicated that the semi-dwarf phenotype was a result of differential splicing of the transcript in br1-Mutag due to interference of Mu-insertion in intron1.

To further validate the br1 candidate gene, Reverse Transcriptase-coupled Polymerase Chain Reaction (RT-PCR) was performed by collecting total RNA samples from 4 week old plants and using Br1 GSPs. A larger size transcript in br1-Mutag as compared to its WT-sibs was detected. Cloning and sequencing of the RT-PCR product allele revealed the presence of 141 bp of Mu-TIR in br1-Mutag transcript indicating an interference of Mu-insertion in its intron1 splicing. Addition of 141 bp of Mu-TIR in the transcript of br1-Mutag allele (starting at position 328 of SEQ ID NO: 16) led to addition of 58 aa (starting at position 110 of SEQ ID NO: 5) and a frame shift and an early stop codon in the coding sequence. RT-PCR expression analysis also showed a complete absence of Br1-transcript in br1-ref mutant indicating that the insertion of a novel retro-transposon (RTE) in br1-ref mutant allele destabilized its transcript. These RT-PCR results also confirmed the functional relationship between the ZmBr1 and the br1 phenotype.

### Example 3

### Characterization of Br1 Gene and Encoded MYB Transcription Factor

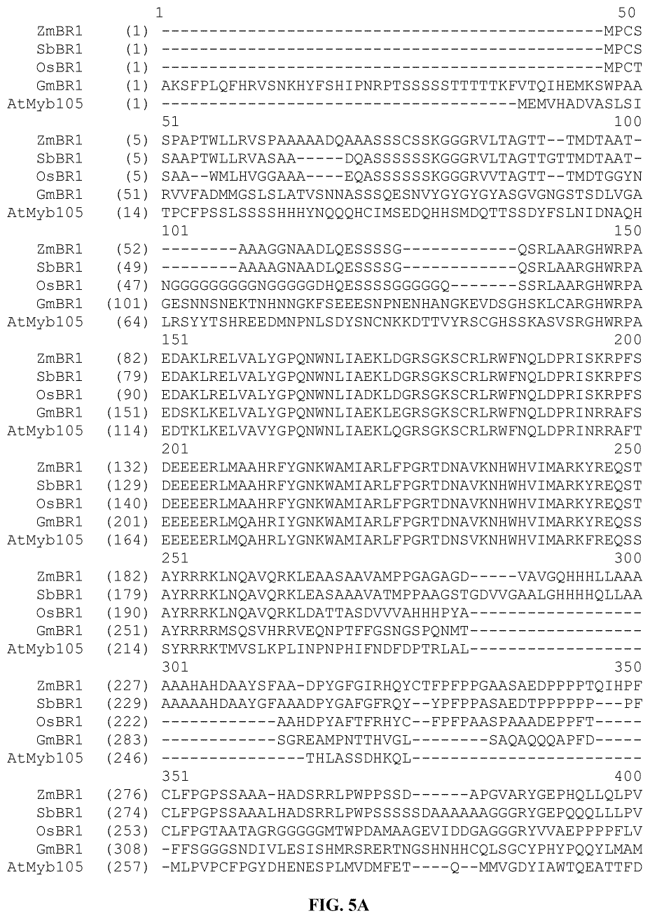

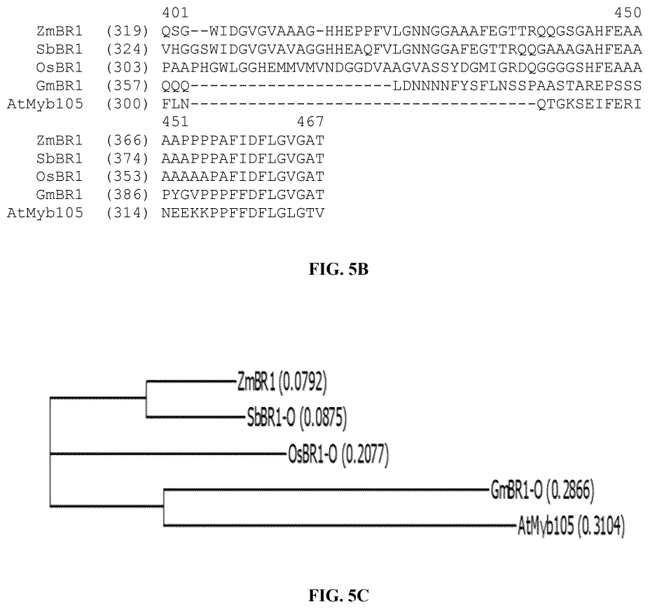

FIG. 5 depicts the expression pattern of the maize Br1 gene in different tissues of an inbred line. The Br1 gene is expressed at a very low level in almost all plant parts with maximum 295 PPM in shoot meristem followed by 96 PPM in immature car, 47 PPM in tassel, and 42 in a stalk sample comprised of nodal plate plus pulvinus plus rind and elongation zone. Similar data for average expression of Br1 was also found in meristematic tissue after combining Lynx MPSS signature and MPSS-Cla with Illumina WgT databases. Taken together, Br1 is least expressed in reproductive tissues such as anther, pollen, silk, embryo, endosperm, and pedicel (see also FIG. 3).

Using Pearson correlation value; r=>0.8), a list of about 30 genes showing similar expression pattern to Br1 were selected to measure and authenticate the gene expression quantitatively. BC3F3 plants of br1-ref in B73 background were used for qRT-PCR. Samples from emerging stalk meristem (SM) tip after removing all leaf whorls were collected from 8 mutants and 8 WT-sib at 4th, 5th, and 6th week development stages. Expression of all 30 genes was quantified as relative to the expression of a reference gene cIF4-gamma, a Transcription Factor. The qRT-PCR for two out of 30 genes did not work. The average expression of Br1 candidate gene, Transcription Regulator HTH, was detected very low in qRT-PCR samples in all three stages as evident from LYNX database, and was close to zero in br1-ref mutant (being null allele) as compared to 0.04 in its WT-sib. Majority of the tested genes were down-regulated in br1 mutant as compared to its WT-sib. Among this class, the Br2 gene (p-glycoprotein1, a membrane transporter) involved in polar transporting of auxin in br2 mutation and other closely related multiple drug resistant protein ABC transporter family protein were significantly down regulated in br1 mutant. Similarity Auxin transporter-like protein2 (LAX2), Auxin response factor5 (ARF5) and Growth regulating factor9 (GRF9) were also down regulated significantly in the br1-ref mutant as compared to its WT-sib. A cell division cycle-associated 7-like protein which might be associated with an increase in the average cell counts in mutant was significantly upregulated in br1 mutant as compared to its WT sib. Similarly, an Auxin efflux carrier component 1b-like protein was also significantly upregulated in br1 mutant indicating that Br1 might be playing a role in auxin stimulus.

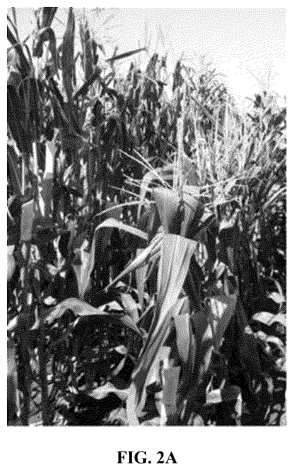

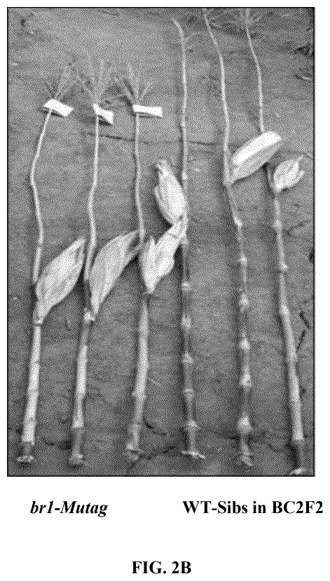

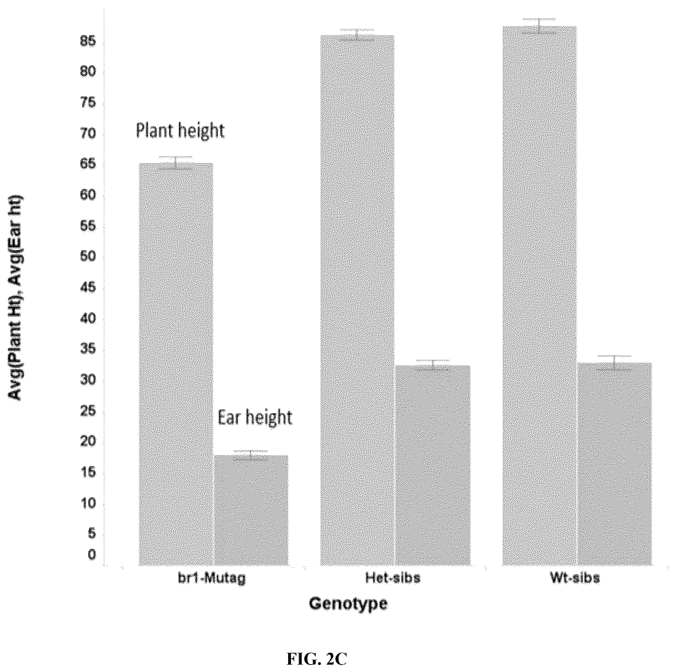

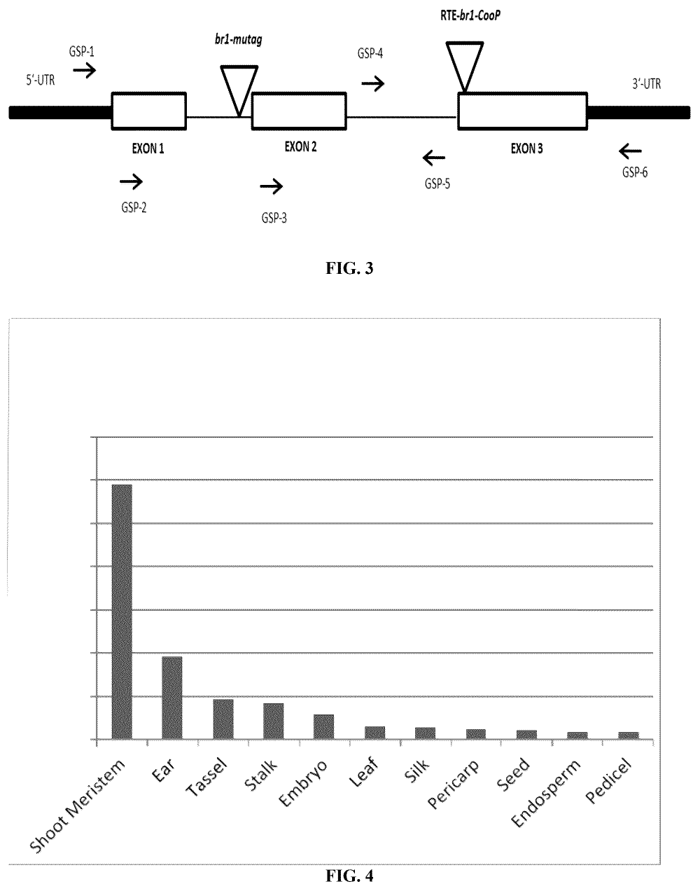

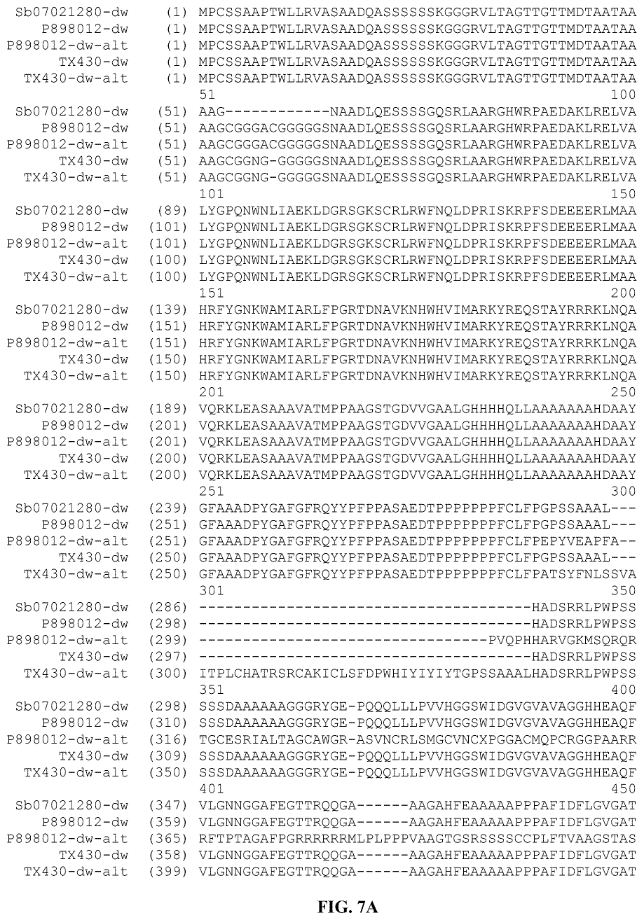

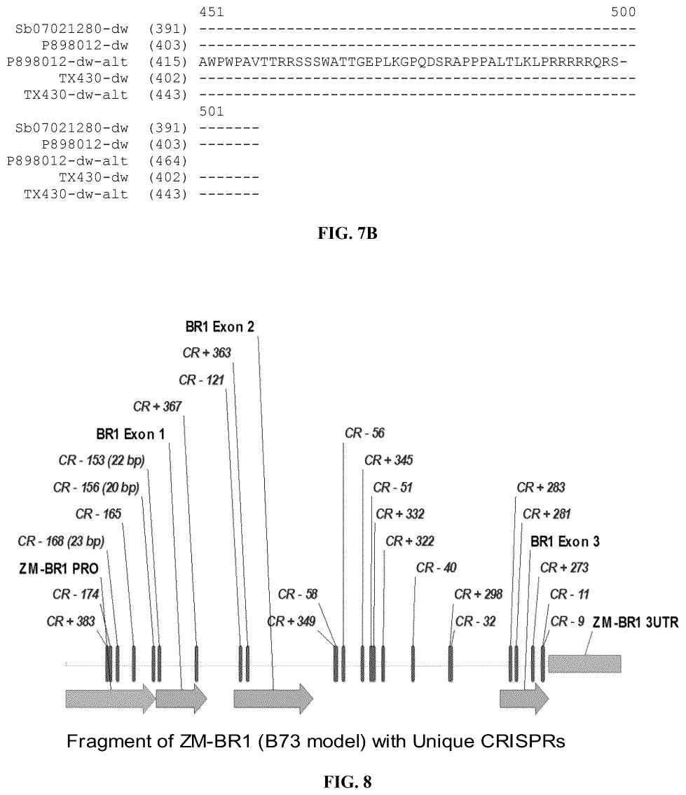

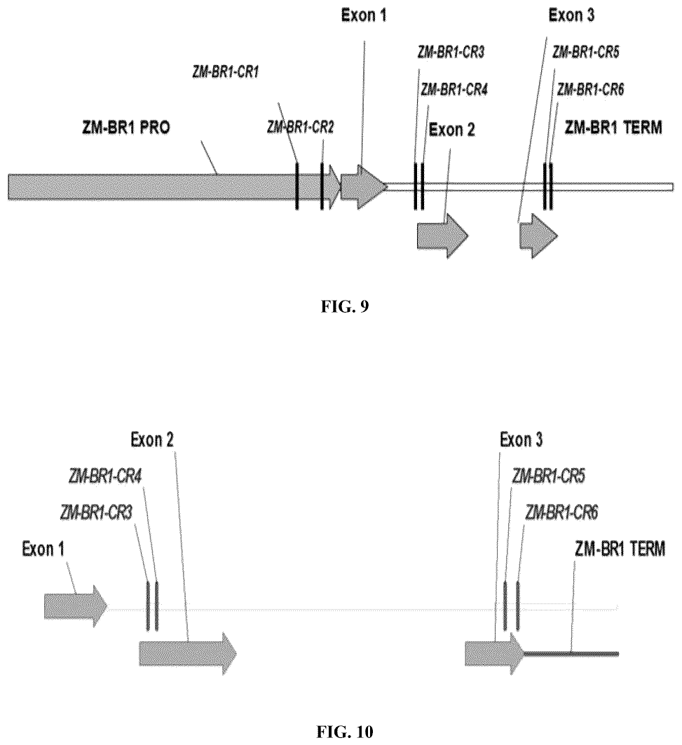

The maize Br1 gene includes of three exons and two introns and the coding region of the Br1 gene is 1,149 bp long (FIG. 2). There are four gene models for the Br1 candidate genes, dpzm01g068810 (SEQ ID NOS: 10-14). Pfam analysis showed that BR1 belongs to MYB-like DNA-binding domain; R2R3-Myb family (PF13921.1). The MYB-super family domain starting from 74 to 183 amino acids in ZmBR1 peptide are highly conserved in both monocot and dicot plant species (FIG. 7A). The ZmBR1 peptide is 83.1% identical to sorghum SbBR1 (Sb07g021280.1) and 41.7% with rice OsBR1 (Os08g33800.1), but only 36.5%, 34.5%, and 27.5% with soybean GmBR1 (Glyma01g05980.1), and Arabidopsis MYB105 (At1g69560.1) proteins, respectively (FIG. 4). The ZmBR1 protein along with sorghum and rice has diverged from dicot soybean and Arabidopsis substantially in the N-terminal signal peptide and the hydrophobic C-terminal sequences. The predicted BR1 protein in maize is 382 amino acids long in comparison to the 390 amino acids in sorghum, 369 amino acids in rice, 402 in soybean, and 330 amino acids in Arabidopsis. Maize BR1 is a monocot specific transcription regulator involved in plant height.

Homolog of the ZmBr1 candidate gene in sorghum (Sb07g021280) was amplified by using gene specific primers (GSPs) from exon2 and exon3. RT-PCR amplified two transcripts, one small size of 620 bp transcript with higher intense product band as expected and another with a larger size in both in TX430 and P898012 lines. The sequence and alignment analyses showed that the differential splicing of intron3 added 123 bp in TX430 cDNA (alternate transcript) and its predicted peptide became 41 amino acids long but was still in frame. However, the differential splicing of intron3 resulted in adding 209 bp in P898012 transcript which lead to addition of 43 aa and early stop codon. The P898012 harbors a mutant allele at Sb7.2 locus, which controls the plant height.

### Example 4

### Evaluation of br1-Mutag Allele

To evaluate br1-Mutag as a weak mutant allele that exhibit plant height differences particularly at flowering stage, homozygous mutant and homozygous WT-sibs of br1-Mutag in Mo17 in BC4F3 generation (here referred as Near Isogenic Lines; NILs) were used and collected stalk meristem tips samples at 4th, 5th, 6th, 7th, and 8th week old plants grown in GH. Both mutants and WT-sib NILs were representing V9, V11, V13, V15, and RI growth stages. RT-PCR analysis was performed using total RNAs from stalk meristems. Two gene specific primers, GSP-157730 and GSP-157726 used in RT-PCR expression were from designed from 5′UTR and exon3 end, respectively. RT-PCR produced three transcripts (labeled as 1, 2, and 3) in br1-Mutag as compared to one normal in its WT-sib NIL at all growth stages except at R1. The br1-Mutag mutant is producing normal transcript relatively in less intensity as compared to two bigger size differential spliced transcripts. Cloning and sequencing analysis of three transcripts of br1-Mutag confirmed that the mutant has normal transcript similar to its WT-sib and its two bigger size transcripts were products of differential splicing of intron 1 by interference of Mutator insertion. No transcript detected in br1-Mutag mutant at RI growth stage indicating that mutant is behaving like a null mutant allele at flowering stage. Weak br1-Mutag allele effects are in part due to low level expression of Br1 gene coupled with production of differential spliced transcripts relatively in substantial quantity at early growth stages which become unstable at flowering.

Expression analysis: Expression of 30 different genes was measured in br1-CooP mutant and its WT-sibs in BC3F3 generation as expression relative to the reference gene (cIF4-gamma) by qRT-PCR. Transcription Regulator HTH, a candidate gene for br1, is expressing at a very low level and br1-CooP mutant was a null allele. A set of genes which were significantly down regulated in br1 mutant as compared to its WT-sib and among those was Zmpgp1, a membrane transporter involved in polar transporting of auxin in br2 mutation and its related multiple drug resistant protein. A cell division cycle-associated 7-like gene was significantly upregulated in mutant which might be associated with an increase in the average cell counts in br1 mutation as compared to its WT sibs. Similarly, an Auxin efflux carrier component 1b-like protein was upregulated in mutant indicating that ZmBr1 HTH transcriptional regulator has role in auxin stimulus.

### Example 5

### Allelic Variation at Br1 Locus and Application for Generating Weaker Alleles of Br1 Mutation

The candidate gene for br1 mutation mapped to c1_192.24 cM with its physical location at 223,645,759-223,649,276 and there are 4 splice variants of the gene model listed in the database. Genotypic variation in 507 out of 600 analyzed maize lines (84%) at the br1 locus is covered by four haplotypes belonging to groups 1, 2, 4, and 6. These haplotypes are present with 0.26 and 0.68 frequencies for groups 1 and 4 in SS germplasm and 0.31, 0.21 and 0.29 frequencies for groups 1, 2, and 6 in NSS germplasm, respectively. Haplotypes in promoter, 5′UTR, exons, introns, and 3′UTR of Br1 gene sequence were detected among all 5 groups and B73 reference sequence. Among these haplotypes, 62 bp deletion in group 2, 94 bp addition in group 1, 10 bp addition in group 1, and two SSRs of various lengths in group 6 were detected at −2639 bp, −2426 bp, −2000 bp, 1805 bp, and −1162 bp positions upstream of ATG, respectively, demonstrating a wide range of haplotypic variation in the ZmBr1 locus. A haplotype of 18 bp (CGCATATGGGTGTCGGCG) (SEQ ID NO: 77) contained an additional sequence in the 5′UTR of Br1 gene, which was present in group 1 as compared to all other groups and B73 reference sequence. Similarly, a 12 bp indel in groups 1 and 4 as compared to groups 1, 6, and B73 and 3 bp addition in groups 2, 6, and 17 in exon1 and 3 bp indel in group 1 in exon2 coding sequence were also detected. One SNP in exon1 in group 4 and three in exon3 were prominent in group1 and 4 as compared to all other groups. A unique haplotype of 118 bp addition in 3′ UTR is present in group 1 only.

### Example 6

### Generation Br1 Mutant Alleles by Genome Editing

Selection of elite lines for transformation and confirmation of Br1 sequence. For genome editing two elite inbred lines (Non-Stiff Stock and Stiff Stock) were selected as targets. The B73 annotated sequence model for Br1 candidate gene (SEQ ID NO: 10) was first confirmed in these two lines by sequencing. Unique target sites identified in B73 gene model by CRISPR Scan tool were further confirmed in both the inbred lines. Many target sites were identified throughout the length of Br1 gene and about six unique target sites, two each in promoter, exon2, and exon3 selected which were conserved in inbred lines (FIG. 7).

Vector construction and gRNA testing. For Br1 candidate gene validation by deletion (SDN1) and insertion of an extra Mu-TIR sequence in intron1 (SDN3), gRNAs using only four out of six selected CR sites (SEQ ID NOS: 32 to 37) were tested, two each from exon2 and exon3, both in inbred lines. Vector construction was done for all four gRNA using CR3, CR4, CR5, and CR6 unique sites (SEQ ID NOS: 34 to 37) and tested for mutation frequencies.

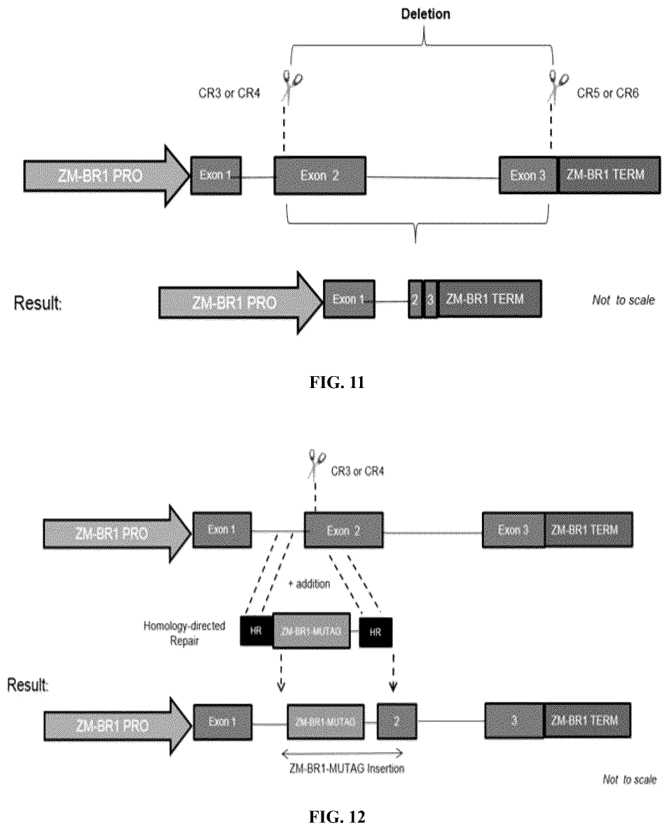

Cas9 SDN1 for deletion. For the Br1 candidate gene validation, deletion of a major part of exon2 and exon3 using a pair of CR4 and CR6 was performed for Cas9 SDN1 approach. A pair of CR4 and CR6 would delete 1894 bp and 1905 bp in the two inbred lines, respectively, because of their differences in intron1 sequence. No unintended ORFs more than 450 bp should be created with perfect repair after deletion done by this pair of CR sites (FIG. 10). Predicted end result would be a Br1 candidate gene with smaller exon2 (104 bp) without second SANT domain and almost whole exon3 gone (except last 33 bp) and is presented in FIG. 10. A total of 250 embryos each of the two inbred lines were transformed using vectors together by bombardment gun. The sequence analysis was completed on ten TO plants. The detail sequence information divided these ten TO plants into two different categories such as plants with perfect deletion as expected and plants with a desired deletion but having extra a few base pair deletion or addition adjacent to their CR sites. A diagrammatic representation based on sequence information of these TO plants with later categories is depicted in FIG. 11. All TO plants were backcrossed with recurrent parents and advanced to T1 generation. Four of ten variants were advanced to T2 following NGS and backcrossing with recurrent parent. Assay was completed on these T2 plants to make sure that these are free from marker, and Cas9 and vector backbone.

A few plants having biallelic deletion showed a reduced internode length phenotype in TO generation. A confirmation of biallelic deletion was done by performing PCR analysis using individual CR4-specific and CR6-specific primers in combination with cross flanking CR site gene specific primers and also using both CR-specific primers together. One perfect biallelic deletion resulted in reduced internode length and thus validating the candidate gene for br1 mutation. Based on sequencing and PCR analyses, four variants each from the two inbred lines were advanced to T2 following sequencing and backcrossing with recurrent parents. Assay was completed on these T2 plants to make sure that these are free from marker, and Cas9 and vector backbone. T2 seed of these variants is being planted for identifying biallelic homozygous plants with br1 gene deletion or truncation. Hybrids are developed using biallelic SDN1 homozygous plants and replicated yield trials are being conducted at various locations.

Cas9 SDN3 for insertion of Mu-TIR fragment. RT-PCR transcript sequence analysis of br1-Mutag showed that the weak phenotype of br1-Mutag allele might be due to the interference of Mu-insertion in splicing of intron1 in its mature transcript. The br1-Mutag had 141 bp extra in its mature transcript which came from TIR sequence of the Mutator (Mu-TIR) and led to a frame shift, addition of 58aa, and an early stop codon in its predicted peptide. For mimicking the br1-Mutag weak mutant phenotype, one gRNA with CR3 (SEQ ID NO: 34) site was used to have a single cut in exon2 and then add 143 bp of Mu-TIR in intron1 by homology directed repair (HDR). A vector with a total 143 bp of Mu-TIR sequence (ZM-BR1-ALT1) along with 500 bp each of left and right homologous arms for homologous recombination was prepared (as shown in FIG. 11). For transformation the same protocol was followed as described in SDN1 section. Total 17 and 18 plants reached to pots in GH and maturity.

Alternatively, GSPs from the Br1 sequence flanked both left and right homologous arms of the ZM-BR1-ALT1 construct were designed and two Mu-TIR specific overlapping primers from 143 bp insertion from the construct were also designed (FIG. 12). Genomic regions of Cas9 SDN3 plants in TO were PCR amplified using these GSPs in combination with Mu-TIR specific primers. When a forward GSP7 (SEQ ID NO: 28) from the 5′ UTR of the Br1 gene is used in combination with a reverse Mu-TIR-a primer (SEQ ID NO: 30), two TO plants (EU_ID #318575270 and #318575410) out of 17 in GXX-INBRED amplified the desired size PCR product. Furthermore, the use of forward Mu-TIR-b primer (SEQ ID NO: 31) in combination with a reverse GSP8 (SEQ ID NO: 29) from end of exon2 confirmed these results indicating that two plants got extra Mu-TIR sequence inserted in the Br1 gene of GXX-INBRED. Similarly, one plant amplified the desired size PCR products. Cloning and sequencing of these PCR products further confirmed that 143 bp of the Mu-TIR fragment has inserted at the right site inside the intron1 which is 15 bp upstream from the intron1-exon2 junction (SEQ ID NO: 21). These TO plants were advanced to T1 by crossing with recurrent parents and PCR amplification and sequence information results were confirmed on progenies of T1 plants (SEQ ID NO: 21). Homozygous plants for Cas9 SDN3 insertion are identified in T2 self progenies in GH and hybrids would be developed using bi-allelic SDN3 homozygous plants in winter nursery and replicated TC yield trials will be conducted at various locations in summer.

### Example 7

### Modification of Stature in Sorghum Through Stable Dw3 Mutagenesis

Dwarfing mutations are used for enhancing harvest index, reducing lodging and increasing yield in many crops. Generally, three independent dwarfing mutations out of four available sorghum dwarf mutants (dw1, dw2, dw3, and dw4) are combined to develop commercial hybrids. dw3 mutation contributes a higher proportion to harvest index and therefore, dw3 mutation is often included in such stacks or trait combination.

However, the sorghum dw3 allele being used in commercial hybrids is unstable due to the presence of a direct repeat of 882 bp in its exon 5 and often reverts to tall (wild-type) by unequal crossing over (Multani et al., (2003) Science. October 3; 302 (5642):81-4). In an aspect, CRISPR Cas9 technology was used to delete the dw3 gene in TX430 transformation background and evaluated the CRISPR CAs9 deletion mutants at dw3 locus (named here as CRISPR-dw3-DO) for various phenotypic traits. These edited dw3-DO mutants are stable and do not revert to wild-type (tall) compared to the original dw3 allele in sorghum. In an aspect, CRISPR-Cas9 was used to edit sorghum genome to engineer changes to the dw3 locus.

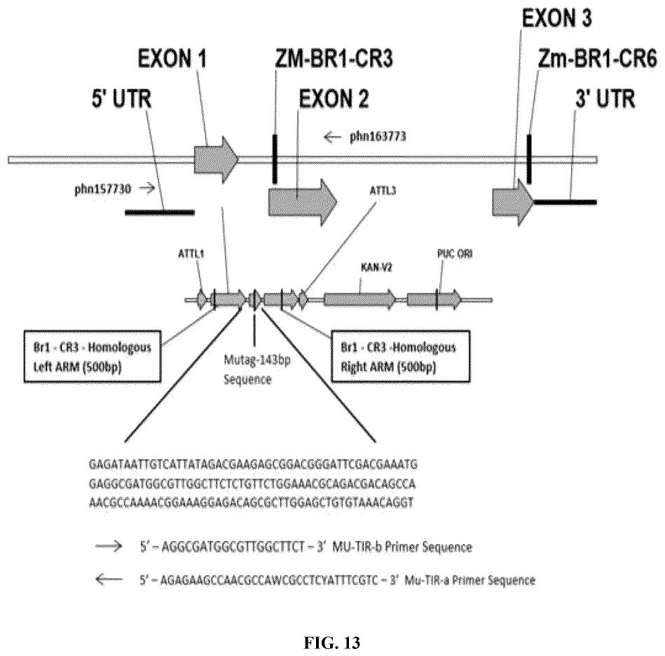

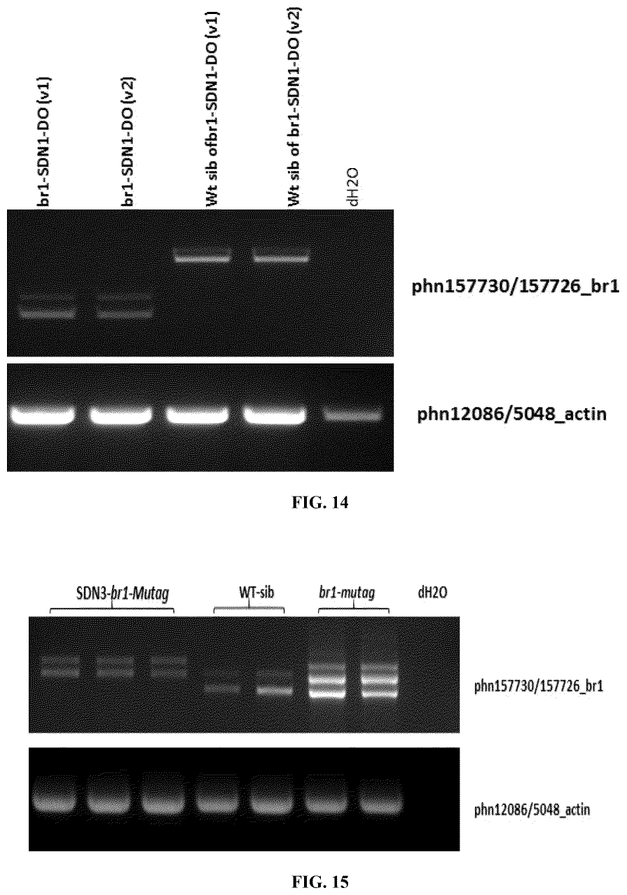

Four gRNAs, two each in 5′UTR and 3′-UTR regions of DW3 gene were designed and tested in TX430 (Table 4; FIG. 13A). Based on efficiency of gRNAs, a pair of DW3-TS1 and DW3-TS3 gRNAs were used to delete the whole dw3-unstable allele in TX430. Two TO plants out of 43 evaluated were selected which were heterozygotes for deletion and advanced by self-pollination to T1 generation and we named here as named here CRISPR-dw3-DO variant1 and CRISPR-dw3-DO variant2. Three plants having variant1 and two in variant2 were identified which were homozygous for the whole dw3 gene deletion and were free from marker, construct, and Cas9. These five plants were self-pollinated and advanced to T2. In a parallel experiment, after genotyping at seedling stage, 53 plants having variant 1 and 62 having variant 2 were transplanted to individual pots and grown to maturity. Data was recorded for ten various phenotypic traits listed in Table 5.

TABLE 4
Sorghum TX430 Dw3 and Dw5 genomic target
site sequence

| | | | SEQ ID |
|---|---|---|---|
| Target Name | gRNA sequence | pam | NO: |
| DW3-TS1 | gccttacaccggtcctcagcga | AGG | 78 |
| DW3-TS2 | gaagacacacgaggctgcct | GGG | 79 |
| DW3-TS3 | gctatatatggtgtatataag | AGG | 80 |
| DW3-TS4 | gttacggtgtgggcaatgtg | CGG | 81 |
| DW5-TS1 | gttctcagggtgaactaaaca | AGG | 82 |
| DW5-TS2 | gaatacatctctcacatatta | GGG | 83 |
| DW5-TS3 | gatacaacacacgttgttgcg | GGG | 84 |
| DW5-TS4 | gaaacacgaggtcttgag | TGG | 85 |

TABLE 5
Phenotypic data on two variants of CRISPR-dw3-DO in T1 generation

| | | | Genotype | |
|---|---|---|---|---|
| Variant | Trait | CRISPR-dw3-DO | Het-Sib | WT-Sib |
| Variant 1 | Avg. Tiller number per plant | 6.50 ± 0.37 | 6.86 ± 0.30 | 6.14 ± 0.31 |
| | Avg. Total leaf number per plant | 14.75 ± 0.27 | 14.95 ± 0.18 | 15.29 ± 0.19 |
| | Avg. PLTHT from first node to apex of the panicle | 100.47 ± 1.65 | 99.90 ± 1.57 | 99.64 ± 2.03 |
| | Avg. PLTHTfrom first node to the base of panicle | 71.69 ± 1.27 | 70.00 ± 1.00 | 70.57 ± 1.46 |
| | Avg. PLTHTfrom the first node to the base of preflag leaf | 51.00 ± 1.32 | 50.48 ± 0.92 | 53.29 ± 1.14 |
| | Avg. Number of Nodes | 12.06 ± 0.21 | 12.13 ± 0.16 | 12.64 ± 0.20 |
| | Avg. Number of Internodes | 11.06 ± 0.21 | 11.13 ± 0.16 | 11.64 ± 0.20 |
| | Avg. Panicle Length | 28.78 ± 0.76 | 29.91 ± 0.75 | 29.07 ± 0.83 |
| | Avg. Days to Flower | 84.63 ± 0.86 | 83.67 ± 0.36 | 84.79 ± 0.77 |
| | Avg. Fresh weight of the Panicle | 99.06 ± 8.39* | 93.68 ± 7.32 | 82.86 ± 7.86 |
| Variant 2 | Avg. Tiller number per plant | 7.09 ± 0.31 | 7.79 ± 0.26 | 7.50 ± 0.48 |
| | Avg. Total leaf number per plant | 16.36 ± 0.28 | 16.62 ± 0.17 | 15.92 ± 0.48 |
| | Avg. PLTHT from first node to apex of the panicle | 98.55 ± 1.51 | 93.88 ± 0.90 | 90.50 ± 2.59 |
| | Avg. PLTHT from first node to base of the panicle | 69.41 ± 1.24 | 67.32 ± 0.60 | 63.79 ± 1.70 |
| | Avg. PLTHT from the first node to the base of preflag leaf | 52.00 ± 0.77 | 50.21 ± 0.68 | 46.92 ± 1.22 |
| | Avg. Number of Nodes | 13.09 ± 0.28 | 13.21 ± 0.15 | 13.00 ± 0.33 |
| | Avg. Number of Internodes | 12.09 ± 0.28 | 12.21 ± 0.15 | 12.00 ± 0.33 |
| | Avg. Panicle length | 29.14 ± 0.63 | 26.56 ± 0.50 | 26.71 ± 1.00 |
| | Avg. Days to Flower | 81.73 ± 0.75 | 80.46 ± 0.30 | 80.92 ± 0.71 |
| | Avg. Fresh weight of the Panicle | 111.37 ± 7.04** | 86.69 ± 4.78 | 70.13 ± 6.20 |

*(p = <0.05%) and
**(p = <0.01)

CRISPR-dw3-DO variants are not expected to have any significant variation on plant height and yield since the unstable dw3 in TX430 (commercial hybrids) is also a null (non-functional) allele. No significant negative effect was recorded in both variants for all ten phenotypic traits observed for T1 plants. Phenotypic data analysis showed both CRISPR-dw3-DO variant1 and CRISPR-dw3-DO variant 2 did not show any significant variation in nine out of ten phenotypic traits recorded as compared to its WT-sibs (Table 5). However, the fresh panicle weight was improved in CRISPR-dw3-DO variant 1 as compared to its WT-sibs (* p=<0.05) and significantly improved in CRISPR-dw3-DO variant 2 (** p=<0.01) as compared to both its heterozygous and WT-sibs (Table 2). Furthermore, the dw3 gene in these CRISPR variants at dw3 locus did not revert to the tall phenotype as observed for other dw3 sorghum allele that were not mutagenized as disclosed herein. Thus, this Example demonstrates that by selectively inactivating or modifying the dw3 locus in sorghum through site-specific nucleotide changes, the dwarf phenotype of sorghum is maintained and not reverted that has been generally observed for native variation of the dw3 mutation.

### Example 8

### Engineering Gibberellic Acid Pathway Components Through Genome Editing for Stature Modification

Gibberellins have been identified as determinants of plant height in many plant species including maize and rice. Mutants such as sd1 in rice, rht-1 in wheat or barley sdw 1 map to genes involved in gibberellin synthesis or signaling. Through CRISPR-cas genome editing nucleic acid guided CAS endonucleases, previously known mutations of GA pathway are introduced into more elite germplasm with minimal genetic drag associated with conventional breeding material. Through CRISPR-cas genome editing nucleic acid guided CAS endonucleases, weaker or stronger alleles of previously known mutations of GA pathway are introduced into more elite germplasm. Through CRISPR-cas genome editing nucleic acid guided CAS endonucleases, new variations of one or more components of the GA pathway are introduced into elite germplasm with minimal genetic drag associated with conventional breeding material. These targets include for example, GA1, GA3, GA4, GA7, GA20-oxidases (GA200x), GA3-oxidases (GA30x) and GA2-oxidases (GA2ox). Disruption of these enzymes through genome editing affects plant stature. GA20ox and GA3ox catalyze oxidations which convert inactive GAs into active GAs (GA20, GA1, GA4) and thus enhance GA responses. GA2ox deactivates GAs by converting GA4 and GA1 into inactive forms. Therefore, methods and compositions are provided that modulate the expression levels, activity levels and a combination thereof of GA these GA biosynthetic pathway that impact plant stature. More specifically, genome edited variants are provided that affect GA biosynthesis, GA signaling and/or a combination thereof.

### Example 9

### Genome Editing of DELLA Proteins and Other GA Regulators for Stature Modification

DELLA proteins are a subfamily of the GRAS superfamily of proteins and play an important role in the negative regulation of GA signaling. DELLA proteins such as D8, D9, and others are suitable targets for generating variations in protein function to alter stature. Through CRISPR-Cas genome editing nucleic acid guided CAS endonucleases, previously known mutations of DELLA proteins are introduced into more elite germplasm with minimal genetic drag associated with conventional breeding material. Through CRISPR-Cas genome editing nucleic acid guided CAS endonucleases, weaker or stronger alleles of previously known mutations of DELLA proteins are introduced into more elite germplasm of plants such as maize, rice, wheat, sorghum and other crop plants.

In addition to DELLAs, feedback regulators of GA biosynthesis such as for example, RSG (Repression of Shoot Growth), a bZIP transcription factor, and its interactors 14-3-3, SCL3 (Scarecrow-like3), another member of the GRAS family and those components that have been identified as GA regulators are targets for genome editing. Therefore, methods and compositions are provided that modulate the expression levels, activity levels and a combination thereof of GA regulators such as DELLA that impact plant stature. More specifically, genome edited variants are provided that affect GA regulation, GA signaling and/or a combination thereof are provided herein.

### Example 10

### Brassinosteroid Pathway Modification Through Genome Editing

Brassinosteroids are a group of steroid hormones that have been identified in many plant species for a variety of functions including stature. Brassinosteroid-deficient mutants have also been a significant source of dwarfism in crops such as barley, e.g., uzu-type barley, which is insensitive to brassinosteroid treatment, has lodging resistance and upright leaf angle; Arabidopsis BRI1; and rice D61, encoding the brassinosteroid receptor.

Through CRISPR-cas genome editing nucleic acid guided CAS endonucleases, previously known mutations of Brassinosteroid pathway are introduced into more elite germplasm with minimal genetic drag associated with conventional breeding material. Through CRISPR-cas genome editing nucleic acid guided CAS endonucleases, weaker or stronger alleles of previously known mutations of Brassinosteroid pathway are introduced into more elite germplasm of plants such as maize, rice, wheat, sorghum and other crop plants. Genome edited variants of Brassinosteroid pathway may exhibit varying degrees of one or more characteristics selected from: shortened upper internodes, shorter grain, upright leaves, delayed flowering time, delayed leaf senescence. In addition to the biosynthetic pathway, perception and signal transduction of the Brassinosteroid pathway are amenable to manipulation using the methods and compositions provided herein for modulating stature. Stronger or weaker alleles of BRI1, BRL1, BRL3 receptors are also suitable candidates for genome editing to improve stature, for example, by reducing plant height. Weaker alleles of the Brassinosteroid biosynthetic enzymes or targeting genes downstream of the major steps of the biosynthesis pathway may be helpful ways to address reducing plant height by modulating Brassinosteroid pathway, wherein the plant height reduction is not severe and semi-dwarf phenotype is obtained.

### Example 11

### Identifying Additional Alleles of Br1 in Germplasm

Examples 1-6 herein describe identification, cloning and characterization of one or more alleles of Br1 in maize and sorghum. Based on the phenotype of Br1 mutants and sequences provided herein, for example, nucleic acid sequences encoding peptides of SEQ ID NOS: 1-9, and nucleic acid sequences of SEQ ID NOS: 10-20, one of ordinary skill in the art can readily design oligonucleotide primer sequences to target genomic loci encoding Br1 peptide and amply a plurality of regions in a population of plants that display varying degrees of dwarfism or dwarf phenotype or shorter stature. Based on the amplified segments and their association with the stature phenotype, novel alleles are identified and characterized. Similarly, a plurality of maize plants such as maize inbred and hybrids are sequenced at the Br1 genomic loci (e.g., a region that includes 5′UTR, regulatory sequences, CDS, introns, exons, 3′UTR) to identify new variations, e.g., Br1 haplotypes at the genomic region that encodes Br1 peptide. Subsequently such new Br1 variations are introduced through conventional breeding or by genome editing such as, for example through CRISPR-Cas guided endonucleases. Similar methodologies are adapted for screening sorghum plants, rice plants, wheat plants and other crops of interest.

### Example 12

### Phenotypic Observation of Genome Edited BR1, BR2 and D8 Maize Plants

Genome-edited variants were generated in either Non-Stiff Stalk (NSS) or Stiff Stalk (SS) inbred backgrounds for BR1, BR2 and D8 genes. In most cases, a pair of guide RNAs targeting the respective loci (e.g., Br1, Br2 and D8) were used in the experiments. The edited plants were identified by PCR and amplicon sequencing, crossed with non-edited recurring parent and the mutations again confirmed in F1 progenies. Plants with the entire regions between the cut sites of the two guides deleted were selected and advanced, although in some cases plants with small IN/DEL-type mutations were maintained. The resulting mutations in each variant are described in the table at a specific sequence level, along with the corresponding guides used. Once the mutations were confirmed, and all transgene components such as Cas9, gRNAs and selectable marker genes were segregated away. The variants were selfed to homozygosity. Phenotyping experiments were carried out with plants both homozygous and heterozygous for the Cas9-derived mutations. The table lists plant and ear heights of the variants relative to the corresponding nulls of each variant, showing stature changes from 34% and up for plant height, and 24% and up for ear height, in homozygous background. In a few cases, edited variants appear to have taller stature, illustrating the wide range of genetic variations and their impact on plant architecture phenotype.

TABLE 6
Genome edited phenotype of maize Br1, Br2 and D8 plants.

| | | | | | Plant Height | | Ear Height | |
|---|---|---|---|---|---|---|---|---|
| | | | Guide | Mutations | Homo- | Hetero- | Homo- | Hetero- |
| Gene | Type | Variant | RNAs | after editing | zygous | zygous | zygous | zygous |
| BR1 | NSS | GV2.7 | BR1-CR4; | −1905 bp | 34% | 103% | No data | No data |
| | | | BR1-CR6 | | | | | |
| BR1 | NSS | GV4.4 | BR1-CR4; | −1905 bp | 48% | 104% | 30% | No data |
| | | | BR1-CR6 | | | | | |
| BR1 | NSS | GV4.13 | BR1-CR4; | −1905 bp | 36% | 96% | No data | No data |
| | | | BR1-CR6 | | | | | |
| BR1 | NSS | GV3.1 | BR1-CR4; | −TT at CR4 | 42% | 98% | 28% | 100% |
| | | | BR1-CR6 | with +T at | | | | |
| | | | | CR6 | | | | |
| BR1 | NSS | GV3.4 | BR1-CR4; | +T at CR4 | 49% | 103% | 38% | No data |
| | | | BR1-CR6 | with +T at | | | | |
| | | | | CR6 | | | | |
| BR1 | NSS | GV2.4 | BR1-CR4; | −13 bp at | 48% | 97% | 29% | 100% |
| | | | BR1-CR6 | CR4 with +A | | | | |
| | | | | at CR6 | | | | |
| BR1 | SSS | GV.2.29 | BR1-CR3 | 143 bp MU | 47% | 100% | 34% | 100% |
| | | | | insertion | | | | |
| BR2 | NSS | GV3.19 | BR2-CR1; | −578 bp | 38% | 94% | 24% | 100% |
| | | | BR2-CR3 | deletion | | | | |
| | | | | (from −579 | | | | |
| | | | | bp + A) | | | | |
| BR2 | NSS | GV3.22 | BR2-CR1; | +T at CR1 | 40% | 101% | 25% | 100% |
| | | | BR2-CR3 | with +T at | | | | |
| | | | | CR3 | | | | |
| BR2 | NSS | GV3.13 | BR2-CR1; | −573 bp (−592 | 38% | 90% | 25% | 100% |
| | | | BR2-CR3 | bp with 19 bp | | | | |
| | | | | BR2 flipped | | | | |
| | | | | & reinserted) | | | | |
| D8 | NSS | GV2.9 | D8-CR2; | −T at CR2 | 100% | 101% | 96% | 100% |
| | | | D8-CR3 | with −9 & 1 | | | | |
| | | | | SNP at CR3 | | | | |
| D8 | NSS | GV2.11 | D8-CR2; | −2 bp at CR2 | 101% | 100% | 100% | 100% |
| | | | D8-CR3 | with −3 at | | | | |
| | | | | CR3 | | | | |
| D8 | NSS | GV2.15 | D8-CR2; | −579 bp with | 38% | 50% | 36% | 100% |
| | | | D8-CR3 | 44 bp D8 | | | | |
| | | | | insertion | | | | |
| D8 | NSS | GV3.15 | D8-CR2; | −23 at CR2 | 94% | 100% | 104% | 100% |
| | | | D8-CR3 | with wildtype | | | | |
| | | | | at CR3 | | | | |
| D8 | NSS | GV4.9 | D8-CR2; | −7 bp at CR2 | 96% | 102% | 101% | 100% |
| | | | D8-CR3 | with +A at | | | | |
| | | | | CR3 | | | | |
| D8 | NSS | GV1.7 | D8-CR6; | −15 bp at | 112% | 106% | 104% | 100% |
| | | | D8-CR7 | CR6 with | | | | |
| | | | | wildtype at | | | | |
| | | | | CR7 | | | | |
| D8 | NSS | GV3.5 | D8-CR6; | +A at CR6 | 125% | 97% | 132% | 100% |
| | | | D8-CR7 | with wildtype | | | | |
| | | | | at CR7 | | | | |
| D8 | NSS | GV3.6 | D8-CR6; | −3 bp at CR6 | 101% | 102% | 107% | 100% |
| | | | D8-CR7 | with +T at | | | | |
| | | | | CR7 | | | | |
| D8 | NSS | GV3.25 | D8-CR6; | −234 bp | 104% | 102% | 102% | 100% |
| | | | D8-CR7 | | | | | |
| D8 | NSS | GV3.1 | D8-CR8; | +T at CR8 | 108% | 105% | 107% | 100% |
| | | | D8-CR9 | with +G at | | | | |
| | | | | CR9 | | | | |
| D8 | NSS | GV3.7 | D8-CR8; | +T at CR8 | 111% | 100% | 103% | 100% |
| | | | D8-CR9 | with −C at | | | | |
| | | | | CR9 | | | | |

Data on plant height was recorded in T2S1 generation in all edits for stature (Table 6). The average plant height of br1-deletion and br1-Frame Shift homozygous plants and truncated C-terminal end of br2 gene in homozygous br2-variant plants was reduced to 50% to 60% as compared to its heterozygous and WT-sib plants whereas the average plant height in br1-Mutag insertion homozygous variant was 60-65% as compared to its heterozygous and WT-sib plants. The RT-PCR expression analysis using GSPs from the flanking 5′-UTR and 3′-UTR sequences of br1 gene further confirmed that the br1-del variant has a smaller size transcript as compared to its WT-sib (FIG. 15) and deletion of 1.9 kb created a null variant. RT-PCR expression and transcript sequence analyses of the br1-Mutag variant confirmed a bigger size and addition of 140 bp in br1-Mutag variant in transcript as compared to its WT-sib. However, a WT-transcript was not detected in addition to a bigger size transcript in br1-Mutag variant which was prevalent in the native br1-Mutag mutant allele due to differential splicing of three differential sliced transcripts including a WT-transcript. This expression analysis established that absence of the WT-sib transcript in br1-Mutag variant made it a strong null allele and reduced its plant height more as compared to the native br1-Mutag mutant allele. Therefore, two separate edits can also be made where gRNAs are from promoter region will delete certain regions of the br1-promoter to reduce WT-transcript expression to get a weak variant at br/locus. These include deleting or mutating an enhancer sequence or a potential expression increasing element.

Therefore, this Example demonstrates that modifying gene loci through genome editing results in useful improvement in agronomic characteristics.

### Example 13

### Cloning, Characterization, Genome Editing of Sorghum dw5 Dwarf Allele to Improve Crop Productivity

Synteny relationship and co-linearity exists between the chromosome 1 of maize and 7 of sorghum through comparative mapping. Both br1 and br2 in corn are on long arm of chromosome 1 with about 20 cM distance apart from each other. The dwarfing locus dw3 used in sorghum hybrids in sorghum is mapped to chromosome 7 and is an ortholog of br2 with 91.28% identity at amino acid level. BLAST analysis using cloned br1 candidate gene as query detected a homologous sequence (Sb07g021280.1) on c7 in sorghum which is 83.5% identical at amino acid level to the maize Br1 polypeptide and about 8 cM away from dw3 locus.

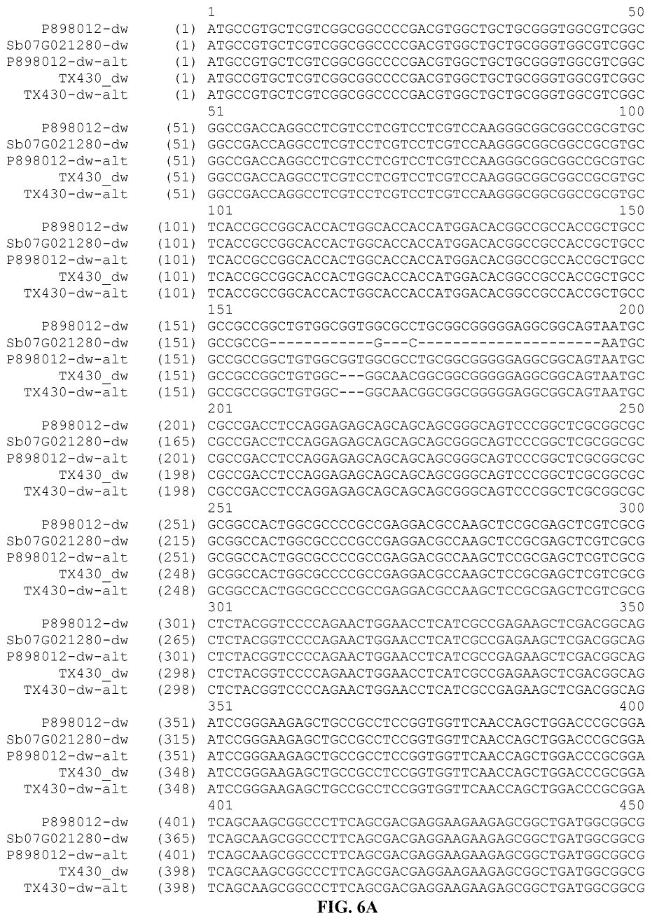

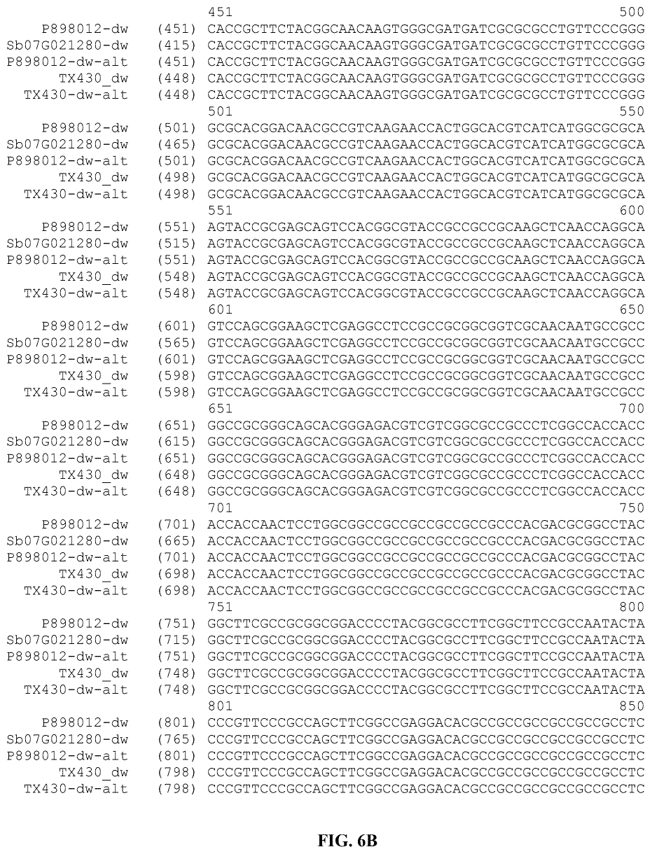

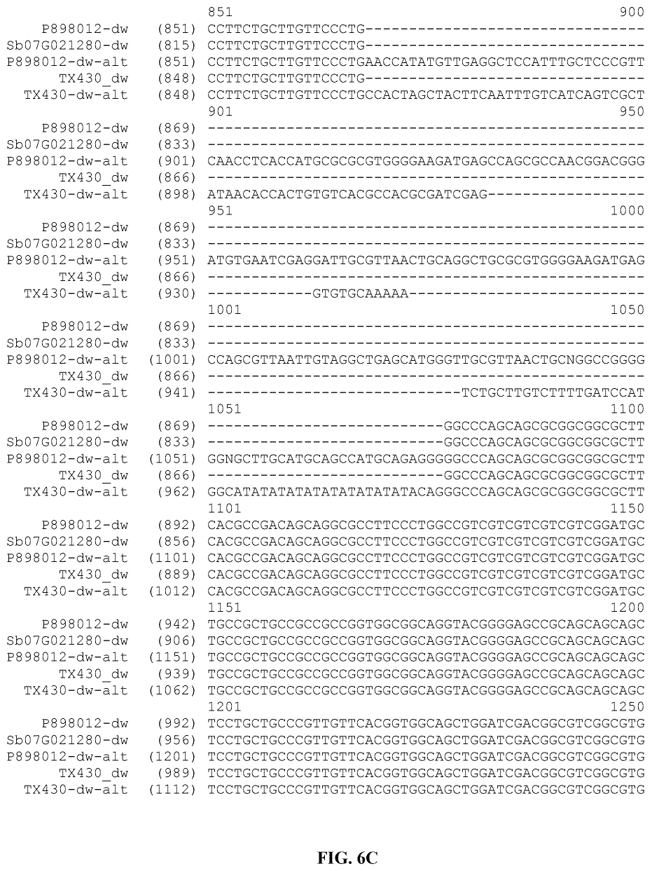

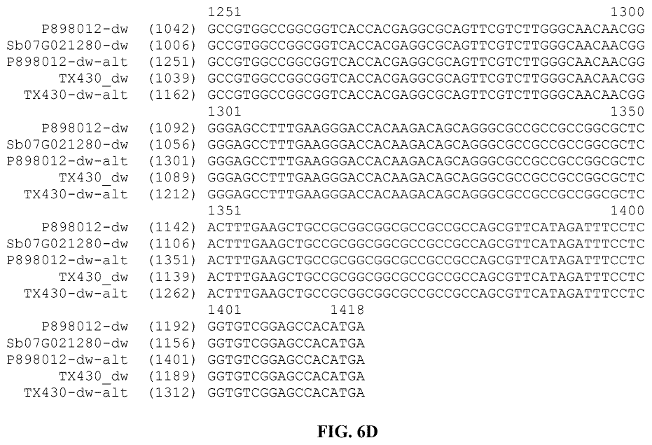

Polymorphism at Sb07g021280.1 locus was determined. A polymorphism between P898012 and TX430 was detected when GSPs from exon2 and exon3 were used. A longer PCR product (˜700-800 bp) in P898012 as compared to TX430 was cloned and sequenced, which revealed the presence of additional 741 bp in intron 2 of P898012 line. Furthermore, RT-PCR and sequencing confirmed an interference of this additional intron sequence in the mature transcript of P898012. Sequences of both normal and differential transcripts in P898012 and TX430 lines are presented in SEQ ID NO: 87 and 88, respectively and multiple alignments of transcripts in FIGS. 6A-6D and their predicted peptides in FIG. 6. Differential splicing in P898012 line resulted in adding 209 bp in its alternate transcript which led to an addition of 43aa and frame shift and truncation of peptide by bringing an early stop codon (SEQ ID NO: 89 & FIG. 6). Whereas the differential spliced transcript of TX430 added 123 bp in its alternate transcript and its predicted peptide is 41 amino acids longer than normal peptide but in frame (SEQ ID NO: 90 & FIG. 6). P898012 harbors a mutant allele at Sb07g02180.1 locus, which controls the plant height and is an ortholog (same function) of corn br1 locus. It was designated dw5 locus and genome editing was initiated for its gene function validation.

TX430 has unstable mutant allele at dw3 locus and WT allele at qHT7.1 locus. Thus, by combining genome edited dw3 mutant allele (stable) and dw5 allele in TX430 results a stable double dwarf (dw3. dw5) which has desirable reduced height as in triple dwarf commercial sorghum hybrids and with reduced reversion to tall (WT) plants.

### Example 14

### Genome Editing of Sorghum Dw3 Allele to Reduce Wild-Type (Tall) Revertants

It has been established that sorghum dw3 allele is unstable and results in wild-type revertants (see e.g., Multani et al., (2003) Science. October 3; 302 (5642):81-4). A variety of techniques are employed to fix the revertant issue found in the existing Sorghum dw3 allele. These approaches include for example, targeted site-specific deletion of one or more of the repeats or an adequate portion of the direct repeats of 882 bp in exon 5 so that the reversion frequency is reduced e.g., less than 10%, or 5% or less when compared to the dw3 allele of sorghum. Another approach is targeted insertion of a heterologous sequence such that the repeats do not get excised during cell cycle which may result in reverting to wild-type (tall) allele. Yet another approach is to create one or more nucleotide modifications at or near the repeat region of the sorghum dw3 genomic loci such that unequal cross-overs which may result in reverting of the dw3 allele to the wild-type allele is reduced. These targeted site-directed mutations/insertions/deletions are engineered using a guided endonuclease e.g., Cas9, cpf1, csm1 and other DNA modification agents. Specifically, guided polynucleotides (e.g. gRNAs) are designed to target one or more genomic regions encoding the Sb Dw3 polypeptide that is at least 95% identical to SEQ ID NO: 95. The guide RNAs, in an embodiment were designed to delete the dw3 allele (see, Example 7) or can also be designed to delete an adequate portion of the repeat such that reversion to wild-type is reduced or even eliminated in subsequent generations. Deletions to the regulatory regions such as the promoter sequences are also contemplated. One or more polynucleotide changes to cause frameshift mutations or to causing premature stop codons that result in non-functional transcripts/polypeptides are also contemplated.

### Example 15

### Sequencing of the Maize Br1 Genomic Region to Identify New Alleles/Polymorphisms of Br1

Targeted sequencing of the Br1 genomic region is performed with samples isolated from a plurality of maize inbred lines. This collection of lines may include germplasm from a variety of sources and geographical regions that for example display a range of stature phenotypes. Based on the sequences provided herein for the maize br1 genomic region, (e.g., SEQ ID NOS: 1-9 and 10-16) primers are designed to selectively amplify a genomic region that encodes or flanks a region for a Br1 polypeptide or a fragment thereof. Whole genome sequencing, deep sequencing, shot gun sequencing or any other available sequencing methodology can also be used to identify allelic variations present in the Br1 genomic region. For example, based on the guidance provided herein, B73 reference genome can be used as a basis to design primers and also as a reference for aligning identified sequences.

Primers are designed through alignment of e.g. B73 sequence of the Br1 gene using commercially available primer design software. These primers can be designed to amplify the entire Br1 genomic gene including about 2k upstream flanking sequence and a 2 kb downstream flanking sequence or 1 kb upstream and 1 kb downstream sequences. PCR amplification is performed using genomic DNA extracted from each line. PCR thermocycling conditions are optimized based on primer length, design and the genomic regions amplified. The amplified products are sequenced and polymorphisms are identified base on the Br1 genomic and downstream sequences obtained. Polymorphisms are identified including both SNPs and INDELs. A polymorphism is defined as a difference in the DNA sequence between any of the sequenced lines compared to the reference sequence or between any of the lines compared to each other. Depending on the location of the polymorphism, amino acid changes resulted from the polymorphisms, if any, are also determined.

A corn plant comprising a new br/allele disclosed herein is crossed with another non-brachytic corn line comprising a desirable trait (e.g., improved yield under drought, cold, heat stress conditions). F1 progeny plants from this cross is assayed for one or more markers identified herein to select for the brachytic (br1) allele. A selected F1 progeny plant is then backcrossed with the parent non-brachytic corn line comprising the desirable trait (recurrent parent). After multiple rounds of backcrossing, a new brachytic corn line is obtained comprising the desirable trait in the recurrent parent elite line.

### Example 16

### Marker Assisted, Genotyping, or Sequencing Based Detection of Additional Br1 Alleles in Maize

In one aspect, this disclosure provides methods of creating a population of corn plants comprising at least one allele associated with a brachytic br1 trait, which methods include the steps of (a) genotyping a first population of corn plants, the population containing at least one allele associated with a brachytic br/trait, wherein the at least one brachytic br1 allele is associated with a marker sequence selected from the group consisting of SEQ ID Nos: 1-8, 10-16, and 70-72 or a fragment thereof; (b) selecting from the first population one or more corn plants containing at least one brachytic br/allele; and (c) producing from the selected corn plants a second population, thereby creating a population of corn plants comprising at least one brachytic allele. In some aspects, these methods comprise genotyping a locus for at least one brachytic allele within about 20 cM, 10 cM, 5 CM, 1 cM, 0.5 CM, or less than 0.5 cM of the marker selected from the group consisting of SEQ ID Nos: 1-8, 10-16, and 70-72 or a fragment thereof.

In one aspect, this disclosure provides methods of selecting a corn plant or seed, the method comprising: (a) isolating a nucleic acid from a corn plant or seed; (b) analyzing the nucleic acid to detect a polymorphic marker associated with a brachytic br/haplotype, the brachytic br/haplotype comprising one or more, two or more, three or more, four or more, five or more, six or more, seven or more, or eight or more brachytic br1 alleles of markers selected from the group consisting of SEQ ID Nos: 1-8, 10-16, and 70-72 or a fragment thereof; and (c) selecting a corn plant or seed comprising the brachytic haplotype. In some aspects, these methods comprise detecting a polymorphic marker within about 20 cM, 10 cM, 5 CM, 1 cM, 0.5 CM, or less than 0.5 CM of the brachytic br1 haplotype. In other aspects, these methods comprise detecting a brachytic haplotype comprising one or more, two or more, three or more, four or more, five or more, six or more, seven or more, or eight or more brachytic br1 alleles of markers selected from the group consisting of SEQ ID Nos: 1-8, 10-16, and 70-72 or a fragment thereof.

In one aspect, this disclosure provides methods of introgressing a brachytic trait into a corn variety, the method comprising: (a) crossing a first corn variety comprising a brachytic br1 trait with a second corn variety not comprising the brachytic trait to produce one or more progeny corn plants; (b) analyzing the one or more progeny corn plants to detect a brachytic allele, wherein the brachytic allele is linked to a marker selected from the group consisting of SEQ ID Nos: 1-8, 10-16, and 70-72 or a fragment/portion thereof; and (c) selecting a progeny corn plant comprising the brachytic br1 allele. In some aspects, these methods comprise detecting a brachytic br1 allele within about 20 cM, 10 cM, 5 CM, 1 cM, 0.5 CM, or less than 0.5 CM of the marker selected from the group consisting of SEQ ID Nos: 1-8, 10-16, and 70-72 or a fragment thereof.

### Example 17

### Sequencing of the Sorghum Dw5 Genomic Region to Identify New Alleles/Polymorphisms of Dw5

Targeted sequencing of the Dw5 genomic region is performed with samples isolated from a plurality of sorghum lines. This collection of lines may include germplasm from a variety of sources and geographical regions that for example display a range of stature phenotypes. Based on the sequences provided herein for the sorghum Dw5 genomic region, (e.g., SEQ ID NOS: 86-92) primers are designed to selectively amplify a genomic region that encodes or flanks a region for a Dw5 polypeptide or a fragment thereof. Whole genome sequencing, deep sequencing, shot gun sequencing or any other available sequencing methodology can also be used to identify allelic variations present in the Dw5 genomic region. For example, based on the guidance provided herein, a sorghum reference genome can be used as a basis to design primers and also as a reference for aligning identified sequences.

Primers are designed through alignment of e.g. reference sequence of the Dw5 gene using commercially available primer design software. These primers can be designed to amplify the entire Dw5 genomic gene including about 2k upstream flanking sequence and a 2 kb downstream flanking sequence or 1 kb upstream and 1 kb downstream sequences. PCR amplification is performed using genomic DNA extracted from each line. PCR thermocycling conditions are optimized based on primer length, design and the genomic regions amplified. The amplified products are sequenced and polymorphisms are identified base on the Dw5 genomic and downstream sequences obtained. Polymorphisms are identified including both SNPs and INDELs. A polymorphism is defined as a difference in the DNA sequence between any of the sequenced lines compared to the reference sequence or between any of the lines compared to each other. Depending on the location of the polymorphism, amino acid changes resulted from the polymorphisms, if any, are also determined.

A sorghum plant comprising a new dw5 allele disclosed herein is crossed with another non-brachytic sorghum line comprising a desirable trait (e.g., improved yield under drought, cold, heat stress conditions). F1 progeny plants from this cross is assayed for one or more markers identified herein to select for the brachytic (dw5) allele. A selected F1 progeny plant is then backcrossed with the parent non-brachytic line comprising the desirable trait (recurrent parent). After multiple rounds of backcrossing, a new brachytic sorghum line is obtained comprising the desirable trait in the recurrent parent elite line.

### Example 18

### Marker Assisted, Genotyping, or Sequencing Based Detection of Additional Dw5 Alleles in Sorghum

In one aspect, this disclosure provides methods of creating a population of corn plants comprising at least one allele associated with a brachytic dw5 trait, which methods include the steps of (a) genotyping a first population of sorghum plants, the population containing at least one allele associated with a brachytic dw5 trait, wherein the at least one brachytic dw5 allele is associated with a marker sequence selected from the group consisting of SEQ ID NOS: 86-92 or a fragment thereof; (b) selecting from the first population one or more sorghum plants containing at least one brachytic dw5 allele; and (c) producing from the selected sorghum plants a second population, thereby creating a population of sorghum plants comprising at least one brachytic dw5allele. In some aspects, these methods comprise genotyping a locus for at least one brachytic allele within about 20 cM, 10 cM, 5 CM, 1 cM, 0.5 CM, or less than 0.5 cM of the marker selected from the group consisting of SEQ ID NOS: 86-92 or a fragment thereof.

In one aspect, this disclosure provides methods of selecting a sorghum plant or seed, the method comprising: (a) isolating a nucleic acid from a sorghum plant or seed; (b) analyzing the nucleic acid to detect a polymorphic marker associated with a brachytic dw5 haplotype, the brachytic dw5 haplotype comprising one or more, two or more, three or more, four or more, five or more, six or more, seven or more, or eight or more brachytic dw5 alleles of markers selected from the group consisting of SEQ ID NOS: 86-92 or a fragment thereof; and (c) selecting a sorghum plant or seed comprising the brachytic dw5 haplotype. In some aspects, these methods comprise detecting a polymorphic marker within about 20 cM, 10 cM, 5 cM, 1 cM, 0.5 CM, or less than 0.5 CM of the brachytic dw5 haplotype. In other aspects, these methods comprise detecting a brachytic haplotype comprising one or more, two or more, three or more, four or more, five or more, six or more, seven or more, or eight or more brachytic dw5 alleles of markers selected from the group consisting of SEQ ID NOS: 86-92 or a fragment thereof.

In one aspect, this disclosure provides methods of introgressing a brachytic dw5 trait into a sorghum variety, the method comprising: (a) crossing a first sorghum variety comprising a brachytic dw5 trait with a second sorghum variety not comprising the brachytic trait to produce one or more progeny sorghum plants; (b) analyzing the one or more progeny corn plants to detect a brachytic allele, wherein the brachytic allele is linked to a marker selected from the group consisting of SEQ ID NOS: 86-92 or a fragment/portion thereof; and (c) selecting a progeny sorghum plant comprising the brachytic dw5 allele. In some aspects, these methods include detecting a brachytic dw5 allele within about 20 cM, 10 cM, 5 CM, 1 cM, 0.5 CM, or less than 0.5 CM of the marker selected from the group consisting of SEQ ID NOS: 86-92 or a fragment thereof.

What is claimed is:

1. A method of reducing plant height, the method comprising introducing one or more nucleotide modifications through a targeted site-directed modification at a genomic locus of a plant, wherein
(a) the genomic locus comprises a polynucleotide involved in gibberellic acid signaling, and
(b) wherein the plant height is reduced compared to a control plant not comprising the one or more introduced genetic modifications.

2. The method of claim 1, wherein the reduction in plant height is in the absence of a substantial reduction in grain yield measure per plant or as a population of plants per unit area.

3. The method of claim 1, wherein the plant height is reduced by about 5% to about 30% compared to the control plant.

4. The method of claim 1, wherein the plant is substantially tolerant to lodging as measured at a single plant level or at an increased planting density, compared to a control plant or control population of plants.

5. The method of claim 1, wherein the plant is maize, and the maize plant comprises an ear, wherein the ear height as measured to the maize plant height is substantially similar or slightly reduced to the ear height measured relative to the control plant height.

6. The method of claim 1, wherein the modifications target the genomic locus such that more than one genetic modifications are present within (a) the same coding region; (b) non-coding region; (c) regulatory sequence; or (d) untranslated region, of an endogenous polynucleotide encoding a polypeptide that is involved in plant height.

7. The method of claim 1, wherein the gibberellic acid signaling genomic locus comprises a polynucleotide that encodes a polypeptide comprising an amino acid sequence that is at least 95% identical to a sequence of SEQ ID NO: 76.

8. The method of claim 1, wherein the gibberellic acid signaling genomic locus comprises a dwarf8 (d8) allele in maize.

9. The method of claim 1, wherein the Dwarf8 (D8) genomic locus comprises an edit in a polynucleotide that encodes a polypeptide comprising an amino acid sequence that is at least 95% identical to a sequence selected from the group consisting of SEQ ID NOS: 73-75, such that the edit results in one or more of the following:
(a) reduced expression of a polynucleotide encoding the D8 polypeptide;
(b) reduced activity of the D8 polypeptide;
(c) generation of one or more alternative spliced transcripts of a polynucleotide encoding the D8 polypeptide;
(d) deletion of one or more domains of the D8 polypeptide;
(e) frameshift mutation in one or more exons of a polynucleotide encoding the D8 polypeptide;
(f) deletion of a substantial portion of the polynucleotide encoding the D8 polypeptide or deletion of the polynucleotide encoding the D8 polypeptide;
(g) repression of an enhancer motif present within a regulatory region encoding the D8 polypeptide; and
(h) modification of one or more nucleotides or deletion of a regulatory element operably linked to the expression of the polynucleotide encoding the D8 polypeptide, wherein the regulatory element is present within a promoter, intron, 3′UTR, terminator or a combination thereof.

10. A semi-dwarf plant comprising a modified genomic locus, wherein the genomic locus comprises one or more mutations compared to a control plant and wherein the genomic locus is characterized by a genomic region that encodes a polypeptide that is at least 95% identical to the amino acid sequence of SEQ ID NO: 76, and wherein the plant is maize and the modification is introduced by a genome editing approach or by marker-assisted breeding approach.

11. The plant of claim 10 wherein the one or more mutations result in a non-functional polypeptide or reduced amount of the polypeptide.

12. A recombinant DNA construct comprising a polynucleotide sequence comprising any of the nucleotide sequences selected from the group of SEQ ID NOs: 73-75, operably linked to at least one heterologous nucleic acid sequence.

13. A plant cell comprising the recombinant construct of claim 12.

14. A guide RNA sequence that targets a genomic locus of a plant cell, wherein the genomic locus comprises a polynucleotide that encodes a polypeptide comprising an amino acid sequence that is at least 90% identical to a sequence of SEQ ID NO: 76, wherein the guide RNA sequence corresponds to a target sequence selected from the group consisting of SEQ ID NOs: 62-69.

15. A recombinant DNA construct that expresses the guide RNA of claim 14.

16. A plant cell comprising the recombinant DNA construct of claim 15.

17. A plant having stably incorporated into its genome the recombinant DNA construct of claim 15.

18. The plant of claim 17 wherein said plant is maize.

19. A seed produced by the plant of claim 17.

20. The plant of claim 17, further comprising a heterologous nucleic acid sequence selected from the group consisting of: a reporter gene, a selection marker, a disease resistance gene, a herbicide resistance gene, an insect resistance gene; a gene involved in carbohydrate metabolism, a gene involved in fatty acid metabolism, a gene involved in amino acid metabolism, a gene involved in plant development, a gene involved in plant growth regulation, a gene involved in yield improvement, a gene involved in drought resistance, a gene involved in increasing nutrient utilization efficiency, a gene involved in cold resistance, a gene involved in heat resistance and a gene involved in salt resistance in plants.
